SEQUENCE LISTING (64 sequences)

SEQ ID NO: 1 - DNA, 35 bp, Artificial Sequence
  Synthetic
ttgacagctagctcagtcctaggtataatgctagc

SEQ ID NO: 2 - DNA, 35 bp, Artificial Sequence
  Synthetic
ttgacggctagctcagtcctaggtacagtgctagc

SEQ ID NO: 3 - DNA, 35 bp, Artificial Sequence
  Synthetic
tttacagctagctcagtcctaggtattatgctagc

SEQ ID NO: 4 - DNA, 35 bp, Artificial Sequence
  Synthetic
ttgacagctagctcagtcctaggtactgtgctagc

SEQ ID NO: 5 - DNA, 35 bp, Artificial Sequence
  Synthetic
ctgatagctagctcagtcctagggattatgctagc

SEQ ID NO: 6 - DNA, 35 bp, Artificial Sequence
  Synthetic
ttgacagctagctcagtcctaggtattgtgctagc

SEQ ID NO: 7 - DNA, 35 bp, Artificial Sequence
  Synthetic
tttacggctagctcagtcctaggtactatgctagc

SEQ ID NO: 8 - DNA, 35 bp, Artificial Sequence
  Synthetic
tttacggctagctcagtcctaggtatagtgctagc

SEQ ID NO: 9 - DNA, 35 bp, Artificial Sequence
  Synthetic
tttacggctagctcagccctaggtattatgctagc

SEQ ID NO: 10 - DNA, 35 bp, Artificial Sequence
  Synthetic
ctgacagctagctcagtcctaggtataatgctagc

SEQ ID NO: 11 - DNA, 35 bp, Artificial Sequence
  Synthetic
tttacagctagctcagtcctagggactgtgctagc

SEQ ID NO: 12 - DNA, 35 bp, Artificial Sequence
  Synthetic
tttacggctagctcagtcctaggtacaatgctagc

SEQ ID NO: 13 - DNA, 35 bp, Artificial Sequence
  Synthetic
ttgacggctagctcagtcctaggtatagtgctagc

SEQ ID NO: 14 - DNA, 35 bp, Artificial Sequence
  Synthetic
ctgatggctagctcagtcctagggattatgctagc

SEQ ID NO: 15 - DNA, 35 bp, Artificial Sequence
  Synthetic
tttatggctagctcagtcctaggtacaatgctagc

SEQ ID NO: 16 - DNA, 35 bp, Artificial Sequence
  Synthetic
tttatagctagctcagcccttggtacaatgctagc

SEQ ID NO: 17 - DNA, 35 bp, Artificial Sequence
  Synthetic
ttgacagctagctcagtcctagggactatgctagc

SEQ ID NO: 18 - DNA, 35 bp, Artificial Sequence
  Synthetic
ttgacagctagctcagtcctagggattgtgctagc

SEQ ID NO: 19 - DNA, 35 bp, Artificial Sequence
  Synthetic
ttgacggctagctcagtcctaggtattgtgctagc

SEQ ID NO: 20 - DNA, 61 bp, Escherichia coli
cctatttgtttatttttctaaatacattcaaatatgtatccgctcatgagacaataaccct

SEQ ID NO: 21 - DNA, 311 bp, Escherichia coli
acttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccat

SEQ ID NO: 22 - DNA, 17 bp, Artificial Sequence
  Synthetic
taatacgactcactata

SEQ ID NO: 23 - DNA, 47 bp, Artificial Sequence
  Synthetic
tcgattcgaacttctgatagacttcgaaattaatacgactcactata

SEQ ID NO: 24 - DNA, 15 bp, Artificial Sequence
  Synthetic
gggagaccgggaatt

SEQ ID NO: 25 - DNA, 26 bp, Artificial Sequence
  Synthetic
agatgattaaagaggagaaattacat

SEQ ID NO: 26 - DNA, 24 bp, Artificial Sequence
  Synthetic
agatgtcacacaggaaaggcccat

SEQ ID NO: 27 - DNA, 22 bp, Artificial Sequence
  Synthetic
agatgtcacacaggacttacat

SEQ ID NO: 28 - DNA, 23 bp, Artificial Sequence
  Synthetic
agatgaaagaggagaaattacat

SEQ ID NO: 29 - DNA, 36 bp, Artificial Sequence
  Synthetic
agatgaatctcatatatcaaatataagcaggatcat

SEQ ID NO: 30 - DNA, 36 bp, Artificial Sequence
  Synthetic
agatgaatctcatatatcaaatatagggtggatcat

SEQ ID NO: 31 - DNA, 33 bp, Artificial Sequence
  Synthetic
agatgatcttaatctagcgtgggagagtttcat

SEQ ID NO: 32 - DNA, 30 bp, Artificial Sequence
  Synthetic
agatgtctagagaaagaagagactcaccat

SEQ ID NO: 33 - DNA, 36 bp, Escherichia coli
gataaatgcttcaatcatgattgaaaaaggaagagt

SEQ ID NO: 34 - DNA, 36 bp, Escherichia coli
aaccttttattcactaacaaatagctggtggaatat

SEQ ID NO: 35 - DNA, 45 bp, Escherichia coli
acccgtttttttggcttttgtgtaactgtaagaaggagatatcat

SEQ ID NO: 36 - DNA, 41 bp, Artificial Sequence
  Synthetic
aaccccttggggcctctaaacgggtcttgaggggttttttg

SEQ ID NO: 37 - DNA, 106 bp, Artificial Sequence
  Synthetic
catctgttttcttgcaagatcagctgagcaataactagcataaccccttggggcctctaaacgggtcttgaggggttttttgctgaaaggaggaactatatccgga

SEQ ID NO: 38 - DNA, 295 bp, Artificial Sequence
  Synthetic
cctagcataaccccgcggggcctcttcgggggtctcgcggggttttttgctgaaagaagcttcaaataaaacgaaaggctcagtcgaaagactgggcctttcgttttatctgttgtttgtcgctgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccgga

SEQ ID NO: 39 - DNA, 297 bp, Artificial Sequence
  Synthetic
cctagcataaaccccttgggttccctctttaggagtctgaggggttttttgctgaaagaagcttcaaataaaacgaaaggctcagtcgaaagactgggcctttcgttttatctgttgtttgtcgctgcggccgcactcgagcaccaccaccaccaccattgagatccggctgctaacaaagcccgaaaggaagctgagttggctgctgccaccgctgagcaataactagcataaccccttggggcctctaaacgggtcttgaggggttttttgctgaaaggaggaactatatccgga

SEQ ID NO: 40 - DNA, 185 bp, Artificial Sequence
  Synthetic
aagcttgcttaagcagaaggccatcctgacggatggcctttttgcgtttctacctagcataaccccttggggcctctaaacgggtcttgaggggttttttggccatctgttttcttgcaagatcagctgagcaataactagcataaccccttggggcctctaaacgggtcttgaggggttttttg

SEQ ID NO: 41 - DNA, 69 bp, Artificial Sequence
  Synthetic
tcaaataaaacgaaaggctcagtcgaaagactgggcctttcgttttatctgttgtttgtcgctgcggcc

SEQ ID NO: 42 - DNA, 45 bp, Artificial Sequence
  Synthetic
ttaagcagaaggccatcctgacggatggcctttttgcgtttctac

SEQ ID NO: 43 - DNA, 48 bp, Artificial Sequence
  Synthetic
ctagcataaccccttggggcctctaaacgggtcttgaggggttttttg

SEQ ID NO: 44 - DNA, 84 bp, Artificial Sequence
  Synthetic
gctgagcaataactagcataaccccttggggcctctaaacgggtcttgaggggttttttgctgaaaggaggaactatatccgga

SEQ ID NO: 45 - DNA, 61 bp, Artificial Sequence
  Synthetic
cctagcataaccccgcggggcctcttcgggggtctcgcggggttttttgctgaaagaagct

SEQ ID NO: 46 - DNA, 63 bp, Artificial Sequence
  Synthetic
cctagcataaaccccttgggttccctctttaggagtctgaggggttttttgctgaaagaagct

SEQ ID NO: 47 - DNA, 10 bp, Artificial Sequence
  Synthetic
catctgtttt

SEQ ID NO: 48 - DNA, 50 bp, Artificial Sequence
  Synthetic
tgtgatctaaaaagagcgacttcggtcgctcttttttttacctgataaaa

SEQ ID NO: 49 - DNA, 99 bp, Artificial Sequence
  Synthetic
ctgtcagaccaagtttactcatatatactttagattgatttaaaacttcatttttaatttaaaaggatctaggtgaagatcctttttgataatctcatg

SEQ ID NO: 50 - protein, 331 aa, Escherichia coli
MTIKVGINGFGRIGRIVFRAAQKRSDIEIVAINDLLDADYMAYMLKYDSTHGRFDGTVEVKDGHLIVNGKKIRVTAERDPANLKWDEVGVDVVAEATGLFLTDETARKHITAGAKKVVMTGPSKDNTPMFVKGANFDKYAGQDIVSNASCTTNCLAPLAKVINDNFGIIEGLMTTVHATTATQKTVDGPSHKDWRGGRGASQNIIPSSTGAAKAVGKVLPELNGKLTGMAFRVPTPNVSVVDLTVRLEKAATYEQIKAAVKAAAEGEMKGVLGYTEDDVVSTDFNGEVCTSVFDAKAGIALNDNFVKLVSWYDNETGYSNKVLDLIAHISK

SEQ ID NO: 51 - protein, 506 aa, Escherichia coli
MIALNTASPQGMQMKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 52 - DNA, 60 bp, Artificial Sequence
  Synthetic
ggcacgtaacgccaaccttttgcggtagcggcttctgctagaatccgcaataattttaca

SEQ ID NO: 53 - DNA, 35 bp, Artificial Sequence
  Synthetic
gtttgatcgcgctaaatactgcttcaccacaagga

SEQ ID NO: 54 - DNA, 110 bp, Artificial Sequence
  Synthetic
ctagaggcatcaaataaaacgaaaggctcagtcgaaagactgggcctttcgttttatctgttgtttgtcggtgaacgctctcctgagtaggacaaatccgccgccctaga

SEQ ID NO: 55 - RNA, 10 nt, Unknown
  Eukaryotic
gccaccaugg

SEQ ID NO: 56 - DNA, 97 bp, Artificial Sequence
  Synthetic
cctatttgtttatttttctaaatacattcaaatatgtatccgctcatgagacaataaccctgataaatgcttcaatcatgattgaaaaaggaagagt

SEQ ID NO: 57 - DNA, 71 bp, Artificial Sequence
  Synthetic
tttacagctagctcagtcctagggactgtgctagcaaccttttattcactaacaaatagctggtggaatat

SEQ ID NO: 58 - DNA, 71 bp, Artificial Sequence
  Synthetic
ctgatggctagctcagtcctagggattatgctagcaaccttttattcactaacaaatagctggtggaatat

SEQ ID NO: 59 - DNA, 71 bp, Artificial Sequence
  Synthetic
tttatggctagctcagtcctaggtacaatgctagcaaccttttattcactaacaaatagctggtggaatat

SEQ ID NO: 60 - DNA, 71 bp, Artificial Sequence
  Synthetic
ttgacagctagctcagtcctagggattgtgctagcaaccttttattcactaacaaatagctggtggaatat

SEQ ID NO: 61 - DNA, 71 bp, Artificial Sequence
  Synthetic
tttatagctagctcagcccttggtacaatgctagcaaccttttattcactaacaaatagctggtggaatat

SEQ ID NO: 62 - DNA, 71 bp, Artificial Sequence
  Synthetic
tttacggctagctcagtcctaggtacaatgctagcaaccttttattcactaacaaatagctggtggaatat

SEQ ID NO: 63 - DNA, 71 bp, Artificial Sequence
  Synthetic
ctgacagctagctcagtcctaggtataatgctagcaaccttttattcactaacaaatagctggtggaatat

SEQ ID NO: 64 - DNA, 59 bp, Artificial Sequence
  Synthetic
tttacggctagctcagccctaggtattatgctagcatggatcacacaggaaaggcccat

(19) United States
(12) Patent Application Publication
(10) Pub. No.: US 2024/0026280 A1
(43) Pub. Date: Jan. 25, 2024

(54) PLASMID ADDICTION SYSTEMS

(71) Applicant: GreenLight Biosciences, Inc., Lexington, MA (US)

(72) Inventors: Himanshu Dhamankar, Arlington, MA (US); Drew S. Cunningham, Winchester, MA (US); Caitlin Macadino, Lexington, MA (US); William Farmer, Concord, MA (US); Susan McAvoy, Lexington, MA (US); Areen Banerjee, Lexington, MA (US); Karthikeyan Ramachandriya, Winchester, MA (US)

(73) Assignee: GreenLight Biosciences, Inc., Lexington, MA (US)

(21) Appl. No.: 18/022,895

(22) PCT Filed: Aug. 23, 2021
(86) PCT No.: PCT/US2021/047111

§ 371 Date: Feb. 23, 2023

Related U.S. Application Data
(60) Provisional application No. 63/091,259, filed on Oct. 13, 2020.
(60) Provisional application No. 63/069,620, filed on Aug. 24, 2020.

(51) Int. Cl. C12N 1/20; C12N 9/02; C12N 15/113; C12R 1/19

(52) U.S. Cl. CPC C12N 1/20; C12N 9/0008; C12N 15/113; C12Y 102/01012; C12R 2001/19

(57) ABSTRACT

Provided herein, in some embodiments, are compositions and methods for a plasmid addiction system based on essential glycolytic genes. Also provided herein, in some embodiments, are compositions and methods for a plasmid addiction system based on an outer membrane efflux protein.

### RELATED APPLICATIONS

This application claims the benefit under 35 U.S.C. § 119(e) of U.S. provisional application No. 63/069,620, filed Aug. 24, 2020, and U.S. provisional application No. 63/091,259, filed Oct. 13, 2020; the contents of each of which are incorporated by reference herein in their entireties.

### BACKGROUND

Recombinant production of nucleic acid-based and protein-based products often occurs in host microbial cells (e.g., Escherichia coli cells). These cells are commonly engineered (e.g., using plasmid-based technology) to produce the desired products in high quantity and with high quality. In order to ensure high quantities of product, a plasmid encoding the desired product must be maintained within the host microbial cell. Plasmid maintenance is routinely enabled by incorporation of antibiotic resistance gene markers into the plasmid. However, incorporation of antibiotic resistance gene markers can be detrimental to the environment if released, as it may promote the emergence of antibiotic resistance in organisms or pathogens, e.g., by taking up and assimilating DNA encoding antibiotic resistance gene markers into their own chromosomes through the process of Horizontal Genetic Transfer (HGT). Therefore, because of the excessive use of antibiotics and regulatory concerns about biological products that comprise antibiotics and DNA encoding antibiotic resistance markers, methods of plasmid maintenance that are antibiotic-free are desired.

### SUMMARY

Disclosed herein are novel antibiotic-free plasmid addiction systems employing a key glycolytic gene (e.g. the prokaryotic gene encoding glyceraldehyde-3-phosphate dehydrogenase, such as the gapA gene from E. coli) as a selection marker for plasmid maintenance in suitable host strains. The inventors discovered that, unlike other plasmid addiction systems that have been described, plasmid addiction systems based on key glycolytic genes (e.g. the gapA gene from E. coli) enable antibiotic-free selection of plasmid-containing microbes in both defined media (e.g. Korz broth as described in Korz et al., 1995, J. Biotechnol. 39:59-65) and complex media (e.g. Luria broth). Plasmid addiction systems that are based on other metabolic pathways (e.g., those based on amino acid metabolism/auxotrophy, or arabinose metabolism/auxotrophy) are typically not expected to enable selection of plasmid-containing microbes in both defined media and complex media. Instead, these alternative plasmid addiction systems would be expected to enable efficient selection in only defined media or often only in defined minimal media. Conversely, the inventors of the present disclosure realized that a plasmid addiction system based on the glycolytic metabolic pathway (e.g., based on gapA) would enable plasmid selection in complex media, such as Luria broth, as well as in defined media. This discovery represents a significant advancement, since propagation of cells for cloning and other plasmid and/or strain management and preparation purposes and preparation of cell banks and final production cell strains is markedly easier in complex media compared to defined media or defined minimal media.

Accordingly, provided herein, in some aspects is a microbial cell lacking or having decreased expression of an endogenous glycolytic gene that encodes a glycolytic enzyme. In some embodiments, the microbial cell comprises a nucleic acid construct comprising an expression cassette that encodes a recombinant glycolytic enzyme, and wherein the microbial cell can grow in a defined medium and/or a complex medium. In some embodiments, the microbial cell cannot grow in the defined medium and/or the complex medium without the nucleic acid construct.

In some aspects, provided herein is a plasmid addiction system comprising (i) a microbial cell comprising a genetic modification of a glycolytic gene that encodes an endogenous glycolytic enzyme, wherein the genetic modification reduces or abolishes the expression of the endogenous glycolytic enzyme; and (ii) a plasmid comprising an expression cassette that encodes a recombinant glycolytic enzyme; wherein the microbial cell cannot grow or propagate without incorporation of the plasmid.

In some embodiments, the genetic modification comprises a mutation, insertion or deletion within the glycolytic gene or a control element of the glycolytic gene, optionally wherein the control element is a promoter or a ribosome binding site. In some embodiments, the recombinant glycolytic enzyme has the same enzymatic activity as the endogenous glycolytic enzyme. In some embodiments, the microbial cell can grow in a defined medium and/or a complex medium if the plasmid is incorporated into the cell.

In some embodiments, the recombinant glycolytic enzyme has the same enzymatic activity as the endogenous glycolytic gene. In some embodiments, the chromosomal DNA of the microbial cell comprises a genetic modification of the endogenous gene or an element controlling the expression of the endogenous gene that decreases the expression of the glycolytic enzyme, optionally wherein the genetic modification is a mutation, insertion or deletion. In some embodiments, the nucleic acid construct is a plasmid, a vector, a cosmid, a bacterial artificial chromosome, a yeast artificial chromosome, a bacteriophage, a viral vector or any other. In some embodiments, the endogenous glycolytic gene encodes a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase. In some embodiments, the recombinant glycolytic enzyme is a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase. In some embodiments, the endogenous glycolytic gene encodes a glycolytic enzyme having glyceraldehyde 3-phosphate dehydrogenase (GAPDH) activity, and wherein the recombinant glycolytic enzyme has GAPDH activity. In some embodiments, the endogenous glycolytic gene encodes a glyceraldehyde 3-phosphate dehydrogenase, and wherein the recombinant glycolytic enzyme is a glyceraldehyde 3-phosphate dehydrogenase. In some embodiments, the glyceraldehyde 3-phosphate dehydrogenase comprises an amino acid sequence of SEQ ID NO: 50.

In some embodiments, the microbial cell is a prokaryotic or eukaryotic cell, optionally wherein the microbial cell is a bacterial cell or a yeast cell. In some embodiments, the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell. In some embodiments, the microbial cell is an Escherichia coli (E. coli) cell, the endogenous glycolytic gene is gapA, and the recombinant glycolytic enzyme is a glyceraldehyde 3-phosphate dehydrogenase.

In some embodiments, the complex media is Luria Broth (LB), Terrific Broth, Super Optimal broth with Catabolite repression (SOC media), or any derivative thereof. In some embodiments, the defined medium is Korz broth, M9 minimal media, or any derivative thereof.

In some embodiments, the nucleic acid construct further comprises a replicon comprising an origin of replication and its control elements. In some embodiments, the replicon is of bacterial origin. In some embodiments, the replicon is the ColE1 replicon, the pUC replicon or is derived from the ColE1, pBR322, pUC, R6K, p15a or pSC101 replicon. In some embodiments, the expression cassette that encodes a recombinant glycolytic enzyme comprises a promoter operably linked to the coding sequence for the recombinant glycolytic enzyme.

In some embodiments, the nucleic acid construct further comprises an expression cassette comprising a sequence of interest, wherein the sequence of interest encodes a RNA product, peptide product or protein product. In some embodiments, the RNA product is a messenger RNA, siRNA, microRNA, guide RNA, a sense strand of a double-stranded RNA, or an antisense strand of a double-stranded RNA. In some embodiments, the nucleic acid construct comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA. In some embodiments, the expression cassette comprising a sequence of interest further comprises a promoter operably linked to the sequence of interest.

In some embodiments, the promoter comprises a nucleic acid sequence set forth in any one of SEQ ID NO: 1-23. In some embodiments, the promoter consists of a nucleic acid sequence set forth in any one of SEQ ID NO: 1-23. In some embodiments, the expression cassette that encodes a recombinant glycolytic enzyme further comprises an initial transcription sequence (ITS) upstream of the coding sequence for the recombinant glycolytic enzyme. In some embodiments, the ITS comprises a nucleic acid sequence set forth in SEQ ID NO: 24. In some embodiments, the ITS consists of a nucleic acid sequence set forth in SEQ ID NO: 24.

In some embodiments, the expression cassette that encodes a recombinant glycolytic enzyme further comprises a 5′UTR comprising a ribosome binding site (RBS) placed upstream of the coding sequence for the recombinant glycolytic enzyme and one or more terminators downstream of the coding sequence for the recombinant glycolytic enzyme. In some embodiments, the RBS comprises a nucleic acid sequence set forth in any one of SEQ ID NO: 25-35. In some embodiments, the RBS consists of a nucleic acid sequence set forth in any one of SEQ ID NO: 25-35. In some embodiments, the one or more terminators comprises a nucleic acid sequence set forth in any one of SEQ ID NO: 36-49. In some embodiments, the one or more terminators consists of a nucleic acid sequence set forth in any one of SEQ ID NO: 36-49.

In some embodiments, the expression cassette comprising a sequence of interest further comprises one or more of the sequence elements selected from the group consisting of: a promoter, an initial transcription sequence, a ribosome binding site, a restriction endonuclease site, and a terminator. In some embodiments, the microbial cell does not comprise an antibiotic resistance gene.

In some embodiments, the plasmid further comprises one or more multicloning sites (MCSs) or unique restriction endonuclease digestion sites. In some embodiments, the plasmid does not comprise an antibiotic resistance gene.

In some aspects, provided herein is a nucleic acid construct comprising an expression cassette comprising a gene encoding an enzyme having glyceraldehyde 3-phosphate dehydrogenase (GAPDH) activity and
(i) one or more multiple cloning sites, and/or
(ii) an expression cassette comprising a sequence of interest encoding an RNA product, peptide product or protein product.

In some embodiments, the nucleic acid construct is a plasmid, a vector, a cosmid, a bacterial artificial chromosome, a yeast artificial chromosome, a bacteriophage, a viral vector or any other. In some embodiments, the gene encoding an enzyme having GAPDH activity is a microbial gapA gene. In some embodiments, the enzyme having GAPDH activity comprises an amino acid sequence of SEQ ID NO: 50. In some embodiments, the nucleic acid construct comprises a first sequence of interest and a second sequence of interest, optionally wherein a first expression cassette comprises the first sequence of interest and a second expression cassette comprises the second sequence of interest. In some embodiments, the first sequence of interest encodes a sense strand of a double-stranded RNA product, and the second sequence of interest encodes an antisense strand of a double-stranded RNA product.

In some embodiments, any one of the expression cassettes further comprises a promoter and/or terminator. In some embodiments, the promoter comprises or consists of a nucleic acid sequence set forth in any one of SEQ ID NO: 1-23. In some embodiments, the promoter is operably linked to an initial transcription sequence (ITS). In some embodiments, the ITS comprises or consists of a nucleic acid sequence set forth in SEQ ID NO: 24. In some embodiments, the promoter is operably linked to a ribosome binding site (RBS). In some embodiments, the RBS comprises or consists of a nucleic acid sequence set forth in SEQ ID NO: 25-35.

In some aspects, provided is a method comprising culturing a microbial cell described herein in the absence of an antibiotic under condition sufficient to produce the nucleic acid construct. In some embodiments, the method produces at least 50% or at least 90% of the total amount of the nucleic acid construct as produced by a control microbial cell comprising an antibiotic resistance marker gene.

In some aspects, provided is a method comprising culturing a microbial cell described herein in the absence of an antibiotic under condition sufficient to produce the RNA product, peptide product or protein product. In some embodiments, the method produces at least 50% or at least 90% of the total amount of the RNA product, peptide product or protein product as produced by a control microbial cell comprising an antibiotic resistance marker gene.

In some aspects, provided herein is a method comprising delivering to a microbial cell a vector comprising a gene encoding glyceraldehyde 3-phosphate dehydrogenase, wherein the microbial cell comprises a genetically modified gene that encodes glyceraldehyde 3-phosphate dehydrogenase, optionally wherein the genetic modification comprises a mutation, insertion or deletion within the gene that encodes glyceraldehyde 3-phosphate dehydrogenase or a control element of the gene, optionally wherein the control element is a promoter or a ribosome binding site.

In some embodiments, the method further comprises culturing the microbial cell in defined medium or in complex medium. In some embodiments, the complex medium is Luria Broth (LB), Terrific Broth, Super Optimal broth with Catabolite repression (SOC media), or any derivative thereof. In some embodiments, the defined medium is Korz broth, M9 minimal media, or any derivative thereof.

In some aspects provided is a kit comprising (i) a nucleic acid construct as described herein; and (ii) a plurality of microbial cells comprising a genetically modified gene that encodes glyceraldehyde 3-phosphate dehydrogenase, optionally wherein the genetic modification comprises a mutation, insertion or deletion.

In some aspects provided is a kit comprising (i) a plasmid comprising an expression cassette that encode a recombinant glycolytic enzyme; and (ii) a plurality of microbial cells comprising a genetic modification of a gene that encodes a glycolytic enzyme, optionally wherein the genetic modification comprises a mutation, insertion or deletion within the glycolytic gene or a control element of the glycolytic gene, further optionally wherein the control element is a promoter or a ribosome binding site.

In some aspects provided is a kit comprising a plurality of microbial cells as described herein. In some embodiments, the plurality of microbial cells are lyophilized or frozen in a cryoprotectant.

Also disclosed herein are novel antibiotic-free plasmid addiction systems employing an outer membrane efflux gene (e.g., the tolC gene from E. coli) as a selection marker for plasmid maintenance in suitable host strains. The inventors discovered that, unlike some other plasmid addiction systems that have been described, plasmid addiction systems based on an outer membrane efflux gene (e.g., the tolC gene from E. coli) enable antibiotic-free selection of plasmid-containing microbes in media comprising a surfactant (e.g., sodium dodecyl sulfate (SDS)). Plasmid addiction systems that are based on other metabolic pathways (e.g., those based on amino acid metabolism or auxotrophy, or arabinose metabolism or auxotrophy) are typically not expected to enable selection of plasmid-containing microbes in media comprising a surfactant. This discovery from the inventors represents a significant advancement, since propagation of cells for cloning and other plasmid and/or strain management and preparation purposes and preparation of cell banks and final production cell strains can be performed with antibiotics and in complex media or defined media.

In some aspects, the disclosure provides a microbial cell lacking or having decreased expression of an endogenous gene that encodes an outer membrane efflux protein, wherein the microbial cell comprises a nucleic acid construct comprising an expression cassette that encodes a recombinant outer membrane efflux protein and an expression cassette that encodes a sequence of interest, and wherein the sequence of interest is expressed when the microbial cell is grown in the presence of a threshold level of a surfactant.

In some aspects, the disclosure provides a plasmid addiction system comprising (i) a microbial cell comprising a genetic modification of a gene that encodes an outer membrane efflux protein, wherein the genetic modification reduces or abolishes the expression of the endogenous outer membrane efflux protein; and (ii) a plasmid comprising an expression cassette that encodes a recombinant outer membrane efflux protein; wherein the microbial cell cannot grow or propagate in a medium containing a threshold level of surfactant without incorporation of the plasmid.

In some aspects, the disclosure provides a nucleic acid construct comprising an expression cassette comprising a gene encoding a protein having tolC activity and (i) one or more multiple cloning sites, and/or (ii) an expression cassette comprising a sequence of interest encoding an RNA product, peptide product or protein product.

In some embodiments, the recombinant outer membrane efflux protein has the same enzymatic activity as the endogenous gene that encodes an outer membrane efflux protein. In some embodiments, the chromosomal DNA of the microbial cell comprises a genetic modification of the endogenous gene or an element controlling the expression of the endogenous gene that decreases the expression of the outer membrane efflux protein. In some embodiments, the genetic modification is a mutation, insertion or deletion.

In some embodiments, the nucleic acid construct is a plasmid, a vector, a cosmid, a bacterial artificial chromosome, a yeast artificial chromosome, a bacteriophage, a viral vector or any other.

In some embodiments, the endogenous gene encodes a tolC, FusA, mexA, mexB, oprM, PpF1, SepA, SepB, SepC, SmeC, OpmE, OpmD, OpmB, or bepC protein. In some embodiments, the outer membrane efflux protein is a tolC, FusA, mexA, mexB, oprM, PpF1, SepA, SepB, SepC, SmeC, OpmE, OpmD, OpmB, or bepC protein. In some embodiments, the endogenous gene encodes a protein having tolC activity, and wherein the recombinant outer membrane efflux protein has tolC activity. In some embodiments, the endogenous gene encodes a tolC protein, and wherein the recombinant outer membrane efflux protein is a recombinant tolC protein. In some embodiments, the recombinant tolC protein comprises an amino acid sequence of SEQ ID NO: 51.

In some embodiments, the microbial cell is a prokaryotic or eukaryotic cell. In some embodiments, the microbial cell is a bacterial cell or a yeast cell. In some embodiments, the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell. In some embodiments, the microbial cell is an Escherichia coli (E. coli) cell, the endogenous gene is tolC, and the recombinant outer membrane efflux protein is a recombinant tolC protein.

In some embodiments, the threshold level of the surfactant is a concentration of surfactant that halts cell growth and/or promotes cell death in a control microbial cell. In some embodiments, the control microbial cell lacks or has decreased expression of an endogenous gene that encodes an outer membrane efflux protein and does not comprise a nucleic acid construct comprising an expression cassette that encodes a recombinant outer membrane efflux protein. In some embodiments, the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

In some embodiments, the nucleic acid construct further comprises a replicon comprising an origin of replication and its control elements. In some embodiments, the replicon is of bacterial origin. In some embodiments, the replicon is the ColE1 replicon, the pUC replicon or is derived from the ColE1, pBR322, pUC, R6K, p15a or pSC101 replicon. In some embodiments, the expression cassette that encodes a recombinant outer membrane efflux protein comprises a promoter operably linked to the coding sequence for the recombinant outer membrane efflux protein. In some embodiments, the promoter comprises a nucleic acid sequence set forth in any one of SEQ ID NO: 1-23. In some embodiments, the promoter consists of a nucleic acid sequence set forth in any one of SEQ ID NO: 1-23. In some embodiments, the expression cassette that encodes a recombinant outer membrane efflux protein further comprises an initial transcription sequence (ITS) upstream of the coding sequence for the recombinant outer membrane efflux protein. In some embodiments, the ITS comprises a nucleic acid sequence set forth in SEQ ID NO: 24. In some embodiments, the ITS consists of a nucleic acid sequence set forth in SEQ ID NO: 24.

In some embodiments, the expression cassette that encodes a recombinant outer membrane efflux protein further comprises a 5′UTR comprising a ribosome binding site (RBS) placed upstream of the coding sequence for the recombinant outer membrane efflux protein and one or more terminators downstream of the coding sequence for the recombinant outer membrane efflux protein. In some embodiments, the RBS comprises a nucleic acid sequence set forth in any one of SEQ ID NO: 25-35. In some embodiments, the RBS consists of a nucleic acid sequence set forth in any one of SEQ ID NO: 25-35.

In some embodiments, the one or more terminators comprises a nucleic acid sequence set forth in any one of SEQ ID NO: 36-49. In some embodiments, the one or more terminators consists of a nucleic acid sequence set forth in any one of SEQ ID NO: 36-49.

In some embodiments, the sequence of interest encodes an RNA product, peptide product or protein product. In some embodiments, the RNA product is a messenger RNA, an siRNA, a microRNA, a guide RNA, a sense strand of a double-stranded RNA, or an antisense strand of a double-stranded RNA. In some embodiments, the nucleic acid construct comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

In some embodiments, the expression cassette comprising a sequence of interest further comprises a promoter operably linked to the sequence of interest. In some embodiments, the promoter comprises a nucleic acid sequence set forth in any one of SEQ ID NO: 1-23. In some embodiments, the promoter consists of a nucleic acid sequence set forth in any one of SEQ ID NO: 1-23. In some embodiments, the expression cassette comprising a sequence of interest further comprises one or more of the sequence elements selected from the group consisting of: a promoter, an initial transcription sequence, a ribosome binding site, a restriction endonuclease site, and a terminator. In some embodiments, the microbial cell does not comprise an antibiotic resistance gene.

In some embodiments, the microbial cell can grow and propagate in a medium containing a surfactant if the plasmid is incorporated into the cell.

In some embodiments, the plasmid comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

In some embodiments, the plasmid further comprises one or more multicloning sites (MCSs) or unique restriction endonuclease digestion sites. In some embodiments, the plasmid does not comprise an antibiotic resistance gene.

In some embodiments, the nucleic acid construct is a plasmid, a vector, a cosmid, a bacterial artificial chromosome, a yeast artificial chromosome, a bacteriophage, a viral vector or any other. In some embodiments, the gene encoding a protein having tolC activity is a microbial tolC gene. In some embodiments, the protein having tolC activity comprises an amino acid sequence of SEQ ID NO: 51.

In some aspects, the disclosure provides a method comprising culturing a microbial cell as described herein in the presence of a threshold level of a surfactant and the absence of an antibiotic under conditions sufficient to produce the nucleic acid construct. In some embodiments, the method produces at least 50% of the total amount of the nucleic acid construct as produced by a control microbial cell comprising an antibiotic resistance marker gene. In some embodiments, the method produces at least 90% of the total amount of the nucleic acid construct as produced by a control microbial cell comprising an antibiotic resistance marker gene.

In some aspects, the disclosure provides a method comprising culturing a microbial cell as described herein in the presence of a threshold level of a surfactant and the absence of an antibiotic under conditions sufficient to produce the RNA product, peptide product or protein product. In some embodiments, the method produces at least 50% of the total amount of the RNA product, peptide product or protein product as produced by a control microbial cell comprising an antibiotic resistance marker gene. In some embodiments, the method produces at least 90% of the total amount of the RNA product, peptide product or protein product as produced by a control microbial cell comprising an antibiotic resistance marker gene.

In some aspects, the disclosure provides a method comprising: delivering to a microbial cell a vector comprising a gene encoding tolC and a gene expressing a sequence of interest, wherein the microbial cell comprises a genetically modified tolC gene, optionally wherein the genetic modification comprises a mutation, insertion or deletion within the tolC gene or a control element of the tolC gene, further optionally wherein the control element is a promoter or a ribosome binding site.

In some embodiments, the threshold level of the surfactant is a concentration of surfactant that halts cell growth and/or promotes cell death in a control microbial cell. In some embodiments, the control microbial cell lacks or has decreased expression of an endogenous gene that encodes an outer membrane efflux protein and does not comprise a nucleic acid construct comprising an expression cassette that encodes a recombinant outer membrane efflux protein. In some embodiments, the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

In some aspects, the disclosure provides a kit comprising: (i) a nucleic acid construct as described herein; and (ii) a plurality of microbial cells comprising a genetically modified tolC gene, optionally wherein the genetic modification comprises a mutation, insertion or deletion.

In some aspects, the disclosure provides a kit comprising: (i) a plasmid comprising an expression cassette that encodes an outer membrane efflux protein; and (ii) a plurality of microbial cells comprising a genetic modification of a gene that encodes an outer membrane efflux protein, optionally wherein the genetic modification comprises a mutation, insertion or deletion within the gene or a control element of the gene, further optionally wherein the control element is a promoter or a ribosome binding site.

In some aspects, the disclosure provides a kit comprising a plurality of any microbial cell as described herein.

In some embodiments, the plurality of microbial cells are lyophilized or frozen in a cryoprotectant.

### BRIEF DESCRIPTION OF THE DRAWINGS

FIGS. 1A-1B provide representative schematics of plasmid selection using an antibiotic selection strategy (FIG. 1A) and a plasmid addiction strategy based on a glycolytic gene (gapA) (FIG. 1B).

FIGS. 2A-2B provide representative schematics of expression plasmids comprising a sequence of interest downstream of an L-arabinose-inducible P_{BAD} promoter and either an antibiotic resistance marker (bla) for plasmid maintenance via antibiotic selection (FIG. 2A) or a gapA gene for plasmid maintenance via a gapA addiction selection strategy in a suitable host (FIG. 2B).

FIGS. 3A-3B provide representative schematics of expression plasmids comprising a T7 RNA polymerase gene downstream of an L-arabinose-inducible P_{BAD} promoter and either an antibiotic resistance marker (bla) for plasmid maintenance via antibiotic selection (FIG. 3A) or a gapA gene for plasmid maintenance via a gapA addiction selection strategy in a suitable host (FIG. 3B).

FIGS. 4A-4C provide representative schematics of template plasmids used for production of double-stranded RNA comprising a sequence of interest (SOI) from two independent expression cassettes that are both under the transcriptional control of a T7 promoter and an initial transcription sequence (ITS). FIG. 4A shows a representative plasmid map containing the bla gene to enable plasmid selection via ampicillin/carbenicillin resistance. FIG. 4B shows a representative plasmid map containing the gapA gene to enable antibiotic-free plasmid selection in a suitable host. FIG. 4C shows a gapA-containing template plasmid and highlights the region upstream of the gapA gene containing a synthetic promoter and a 5′ untranslated region (UTR) containing a ribosome binding site (RBS) used to drive the expression of the exogenous gapA marker. Different promoters and RBSes can be used in combination.

FIGS. 5A-5C provide graphs showing the effect of gapA loss on growth of E. coli in different media conditions. FIG. 5A shows that E. coli lacking endogenous gapA (GL18-134) expression are incapable of growing in defined minimal media (Korz media) or complex media (Luria Broth (LB)) containing carbon sources that require the gapA gene for catabolism via glycolysis, but are capable of growing in defined media when supplemented with carbon sources (glycerol and succinate) that obviate the need for the gapA gene. FIG. 5B shows that the addition of an exogenous plasmid for gapA expression can rescue the growth of E. coli lacking endogenous gapA (GL18-135) in defined minimal media containing carbon sources that require the gapA gene and glycolysis (Korz media). FIG. 5C shows that the addition of an exogenous plasmid for gapA expression can rescue the growth of E. coli lacking endogenous gapA (GL18-135) in complex media containing carbon sources that require the gapA gene and glycolysis (LB).

FIGS. 6A-6C provide graphs demonstrating the effective production of recombinant proteins in E. coli cells using a gapA plasmid addiction strategy. FIGS. 6A-6B shows that E. coli lacking endogenous gapA produce high levels of recombinant proteins expressed from a plasmid comprising an exogenous gapA gene (GL18-135; unARMed). Protein expression in GL18-135 cells was comparable to protein expression in E. coli cells containing the bla antibiotic resistance marker gene (GL17-195; ARMed). FIG. 6C shows data that GL18-135 cells and GL17-195 cells grow and express recombinant proteins at similar rates, as demonstrated by their dry cell weights and protein expression levels (in g/L) over time.

FIGS. 7A-7C provide graphs showing the production of plasmid DNA in E. coli cells using either an ampicillin/carbenicillin resistance selection system (ARMed) or a gapA plasmid addiction strategy (unARMed). FIG. 7A shows plasmid DNA yield from E. coli cells lacking endogenous gapA that are expressing a plasmid comprising a variable gene-of-interest and an exogenous gapA gene. FIG. 7B shows the growth curves of E. coli cells lacking endogenous gapA that are expressing a plasmid comprising a variable gene-of-interest and an exogenous gapA gene. FIG. 7C shows plasmid DNA yields of the ARMed strain and the unARMed variants.

FIG. 8 provides a representative schematic of plasmid selection using a plasmid addiction strategy based on a gene expressing an outer membrane efflux protein (tolC).

FIGS. 9A-9B provide representative schematics of expression plasmids comprising a sequence of interest downstream of an L-arabinose-inducible P_{BAD} promoter and either an antibiotic resistance marker (bla) for plasmid maintenance via antibiotic selection (FIG. 9A) or a tolC gene for plasmid maintenance via a tolC addiction selection strategy in a suitable host (FIG. 9B).

FIGS. 10A-10C provide representative schematics of template plasmids used for production of double-stranded RNA comprising a sequence of interest (SOI) from two independent expression cassettes that are both under the transcriptional control of a T7 promoter and an initial transcription sequence (ITS). FIG. 10A shows a representative plasmid map containing the bla gene to enable plasmid selection via carbenicillin resistance. FIG. 10B shows a representative plasmid map containing the tolC gene to enable antibiotic-free plasmid selection in a suitable host. FIG. 10C shows a tolC-containing template plasmid and highlights the region upstream of the tolC gene containing a synthetic promoter and a 5′ untranslated region (UTR) containing a ribosome binding site (RBS) used to drive the expression of the exogenous tolC marker. Different promoters and RBSes can be used in combination.

FIG. 11 provides a graph showing the effect of tolC loss on growth of E. coli in the presence of low concentrations of SDS (0.005%; 50 mg/L) and subsequent rescue of the ΔtolC phenotype by introduction of tolC on a recombinant plasmid.

FIG. 12 provides a graph showing the production of plasmid DNA encoding dsRNAs of interest in E. coli cells using either a carbenicillin resistance selection system (ARMed: GL18-020) or a tolC plasmid addiction strategy (UnARMed: GL18-196, GL18-197, GL18-198, GL18-199, GL18-200).

### DETAILED DESCRIPTION

The present disclosure provides, in some aspects, methods and compositions for plasmid addiction systems (e.g., plasmid addiction systems that utilize enzymes of the glycolytic pathway and plasmid addiction systems that utilize genes expressing an outer membrane efflux protein). In some embodiments, the plasmid addiction systems described herein enable plasmid retention in microbes without requiring the need for antibiotics or DNA sequences encoding antibiotic resistance markers.

The present invention describes a plasmid addiction strategy that involves the transfer of a gene encoding a key microbial glycolytic enzyme (e.g., gapA) to a plasmid that is to be maintained within bacterial cells (e.g., E. coli cells) that have been engineered to have reduced or eliminated expression of a gene expressing that key microbial glycolytic enzyme. Such a configuration requires the cell having reduced or eliminated endogenous expression of the glycolytic enzyme (e.g., a cell lacking an endogenous gapA gene (gapA-deficient E. coli)) to maintain the plasmid to remain viable in both complex and defined minimal media that are commonly used in industry. Loss of the plasmid would cause the cell having reduced or eliminated endogenous expression of the glycolytic enzyme (e.g., gapA-deficient E. coli) to stop growing and become diluted out by cells that continue to retain the plasmid after a few generations.

The present invention further describes a plasmid addiction strategy that involves the transfer of a gene expressing an outer membrane efflux protein (e.g., tolC) to a plasmid that is to be maintained within bacterial cells (e.g., E. coli cells) that have been engineered to have reduced or eliminated expression of a gene expressing that outer membrane efflux protein. Such a configuration requires the cell having reduced or eliminated endogenous expression of the outer membrane efflux protein (e.g., a cell lacking an endogenous tolC gene (tolC-deficient E. coli)) to maintain the plasmid to remain viable in the presence of a surfactant. Loss of the plasmid would cause the cell having reduced or eliminated endogenous expression of the outer membrane efflux protein (e.g., tolC-deficient E. coli) to stop growing and become diluted out by cells that continue to retain the plasmid after a few generations.

### Definitions

While the following terms are believed to be well understood by one of ordinary skill in the art, the following definitions are set forth to facilitate explanation of the presently disclosed subject matter.

The terms “defined media”, “defined medium”, “chemically defined media” and “chemically defined medium” refer to culture media or medium which are typically prepared using chemically pure biochemicals, inorganic salts and ingredients whose chemical composition is known. As a result, the chemical composition of defined media is generally precisely known.

The terms “defined minimal media”, “minimal media,” “defined minimal medium,” or “minimal medium,” as used herein, generally refer to a defined culture media or medium for microbial cells that is nutritionally poor and consists of the minimal necessities for growth of said microbial cells. In some embodiments, minimal necessities consist of inorganic salts, a simple carbon source (e.g. a monosaccharide such as glucose or glycerol), a simple inorganic nitrogen source (e.g. an ammonium salt) and water. In some embodiments, the carbon source(s) within a defined minimal medium are components of the glycolytic pathway. In some embodiments, a minimal medium is Korz broth, e.g., media as described in or derived from media as described in Korz et al., 1995, J. Biotechnol. 39:59-65. In some embodiments, a minimal medium is M9 minimal medium (e.g., as described in Cold Spring Harbor Protocols) or is derived from M9 minimal medium.

The terms “complex media” or “complex medium,” as used herein, generally refer to a culture medium for microbial cells that is nutritionally rich and includes at least one crude, impure or complex composition (e.g., a composition having multiple components whose chemical structure and/or proportions are not precisely known (e.g., yeast extract, blood, beef extract)). Complex media may comprise inorganic salts, one or more carbon sources, water, and at least one complex composition that serves as a source of amino acids and/or nitrogen and/or carbon. In some embodiments, the carbon source(s) within a complex medium are components of the glycolytic and/or gluconeogenic pathways. In some embodiments, a complex medium is Luria Broth (LB) or media derived from LB, Terrific Broth or media derived from TB, or Super Optimal broth with Catabolite repression (SOC media) or media derived from SOC.

The terms “nucleic acid” or “nucleic acid molecule,” as used herein, generally refer to deoxyribonucleic acid (DNA) or ribonucleic acid (RNA). A nucleic acid may be single-stranded or double-stranded. The nucleotide monomers in the nucleic acid molecules may be naturally-occurring nucleotides, modified nucleotides or combinations thereof. Modified nucleotides, in some embodiments, comprise modifications of the sugar moiety and/or the pyrimidine or purine base.

The terms “transcription” or “RNA transcription” generally refer to the process by which RNA transcripts are synthesized by an RNA polymerase that is capable of polymerizing ribonucleoside triphosphates using a nucleic acid molecule (DNA or RNA) as a template, either in vivo or in vitro.

The terms “template” or “transcription template” or “template for transcription” generally refer to a nucleic acid sequence (DNA or RNA) that serves as a template for an RNA polymerase to make RNA transcripts via the process of transcription. The template specifies the sequence of the RNA transcripts that are synthesized by the RNA polymerase. The RNA polymerase synthesizes RNA transcripts by moving along the template strand of the template nucleic acid molecule and adding ribonucleotide triphosphates complementary to the template (DNA or RNA) strand, to a growing RNA transcript. The template may be DNA or RNA. In some embodiments, the template is single-stranded or double-stranded. In most living organisms, transcription is carried out by RNA polymerases using double stranded DNA molecules (chromosomal DNA) as the template in cells to synthesize mRNA. In some embodiments, in vitro transcription utilizes synthetic partially double stranded DNA templates to be transcribed by DNA-dependent RNA polymerases. In some embodiments, a template is a linear molecule. In some embodiments, a template is circular. A template may contain additional elements other than those necessary for expression of RNA transcripts. Additionally, in vivo and/or in vitro transcription from single-stranded RNA by RNA-dependent RNA polymerases is also possible (e.g. as in the case of some RNA viruses). The terms “template” or “transcription template” or “template for transcription” may, in some embodiments, refer either to a specific nucleic acid sequence of a segment of a double-stranded DNA molecule or to an entire DNA molecule that contains a nucleic acid sequence to be transcribed.

The term “sequence of interest” generally refers to a specific nucleic acid sequence (e.g., a specific nucleic acid sequence present within a template) that is part of an RNA or DNA molecule. In some embodiments, a sequence of interest refers to the nucleic acid sequence that is a part of a DNA template or product. Thus, in some embodiments, a sequence of interest is a segment of the DNA template that encodes a specific nucleic acid sequence of an RNA product. In some embodiments, a sequence of interest is the sequence incorporated into an RNA transcript or RNA product produced via transcription. In other embodiments, a sequence of interest is a segment of the DNA template that encodes a protein of interest (e.g., a gene encoding an enzyme). In some embodiments, a sequence of interest may be a part of an expression cassette. In some embodiments, a sequence of interest is a nucleic acid sequence that is a part or whole of an RNA transcript or product. In some embodiments, a sequence of interest is a gene that encodes a specific RNA transcript or a peptide or protein product. In some embodiments, a sequence of interest is a gene that is incorporated into a nucleic acid construct (e.g., a plasmid or an expression cassette) to allow expression of the desired RNA transcript and/or peptide and/or protein products encoded by the gene.

The terms “sense” and “antisense” generally refer to the individual strands in double stranded DNA or RNA molecules. Accordingly, the term “sense strand” as used herein may refer to the nucleic acid sequence of the coding strand of a double-stranded DNA molecule. In some embodiments, the term “sense strand” refers to a nucleic acid sequence of a segment of or whole of a mRNA transcript produced in vivo or in vitro. The term “sense strand” may also refer to one strand of a double-stranded RNA molecule. Furthermore, the term “antisense strand” may refer to the nucleic acid sequence of a part of or whole of the template strand of a double-stranded DNA that is transcribed to produce mRNA in a given organism. Alternatively, the term “antisense strand” may refer to the nucleic acid sequence of an RNA strand that is complementary to a part or all of an mRNA transcript produced in the cells of a given organism. Further, the term “antisense strand” may refer to a strand of RNA complementary to the sense strand in a double-stranded RNA molecule.

The term “expression cassette” generally refers to a nucleic acid sequence that serves as a template for expression of an RNA transcript of interest via transcription and is minimally composed of a promoter operably linked to a nucleic acid sequence encoding the RNA molecule to be expressed. In some embodiments, an expression cassette refers to a DNA sequence that serves as a template for expression of an RNA transcript or product. In some embodiments, the expression cassette further comprises one or more of the following elements: (1) an initial transcription sequence (ITS) placed immediately downstream of the promoter, e.g., to enhance transcription of the RNA transcripts of interest and such that it is present at the 5′ end of each transcript; (2) a 5′-untranslated region (5′UTR) comprising a ribosome binding site (RBS) that, when incorporated into the resultant RNA transcript assists with translation into a protein, if the transcript encodes a protein; (3) a reverse complement of the ITS (ITS-RC), (4) one or more restriction endonuclease sites; and/or (5) one or more transcriptional terminator sequences. An expression cassette may thus allow the expression of RNA transcripts as products of interest (e.g. siRNAs, shRNAs, sgRNAs, mRNAs, tRNAs etc.) or may further allow expression of a peptide or protein product via translation of the expressed RNA transcripts, when the RNA transcripts are mRNA transcripts encoding a peptide or protein.

The terms “construct”, “nucleic acid construct”, “expression construct”, or “vector” generally refer to a DNA molecule which includes one or more expression cassettes for the expression of an RNA transcript (e.g. mRNA, siRNA, a strand of a dsRNA etc.) via transcription by an RNA polymerase. In some embodiments, when the RNA transcript is a messenger RNA (mRNA), the expression cassette ultimately encodes a protein of interest. A construct may include additional elements that are not critical for expression of the RNA transcript, but are essential for ensuring its own replication, maintenance, stability etc. in vivo or in vitro. For example, a construct may be a plasmid with one or more expression cassettes that further comprises an origin of replication and a selection marker (e.g., a gapA gene) for its replication and maintenance in a suitable host (e.g., a gapA-deficient bacterial cell). In some embodiments, the chromosome of an organism that has been modified by integrating one or more expression cassettes, to allow expression of the RNA transcripts may comprise a construct. Non-limiting examples of constructs include viral vectors (e.g., adeno-associated viral vectors), plasmids, cosmids, plastomes, bacteriophages, artificial chromosomes, natural genomes with integrated expression cassettes, or linear DNA molecules.

The term “initial transcription sequence (ITS)” generally refers to a nucleic acid sequence comprising the first several nucleotides (e.g., 1-15 nucleotides) of the transcribed sequence on the DNA template, immediately downstream of the promoter.

The term “ARMed” refers to an organism, cell, chromosome or nucleic acid constructs (e.g., plasmid) comprising an antibiotic resistance marker (ARM) gene (e.g. the bla gene encoding β-lactamase that confers resistance to ampicillin and carbenicillin). Conversely, the term “UnARMed” refers to an organism, cell, chromosome or nucleic acid constructs (e.g. plasmid) that does not comprise an antibiotic resistance marker gene or otherwise have or confer resistance to antibiotics.

The term “surfactant” refers to amphiphilic molecules (i.e., molecules having a hydrophobic group and a hydrophilic group) that lower the surface tension of a liquid. In some embodiments, a surfactant is a detergents, wetting agents, emulsifiers, or dispersants. In some embodiments, a surfactant is sodium dodecyl sulfate (SDS), cetyl trimethylammonium bromide, Triton X-100, 3-[(3-cholamidopropyl)dimethylammonio]-1-propanesulfonate (CHAPS), nonyl phenoxypolyethoxylethanol (NP-40), octyl thioglucoside, octyl glucoside or dodecyl maltoside. In some embodiments, a threshold level of a surfactant is a minimum concentration of surfactant that halts cell growth and/or promotes cell death in a control microbial cell (e.g., a microbial cell deficient in an outer membrane efflux protein). In some embodiments, a minimum concentration of surfactant that halts cell growth and/or promotes cell death in a control microbial cell is 10, 20, 30, 40, or 50 mg/L surfactant (e.g., SDS).

### Glycolytic Genes

The glycolytic pathway, also known as glycolysis, is a catabolic pathway that constitutes a set of reactions that are part of the central carbon metabolism in nearly all living organisms. The glycolytic pathway allows the breakdown of glucose into pyruvate and is accompanied with the release of energy in the form of ATP. A glycolytic gene encodes an enzyme that carries out a reaction that is part of the glycolytic pathway. In some embodiments, a glycolytic gene encodes an enzyme selected from the group consisting of a hexokinase, a glucose phosphate isomerase, a phosphofructokinase, an aldolase, a triosephosphate isomerase, a glyceraldehyde 3-phosphate dehydrogenase, a phosphoglycerate kinase, a phosphoglycerate mutase, an enolase, a phosphoenolpyruvate carboxylase, a pyruvate carboxylase and a pyruvate kinase.

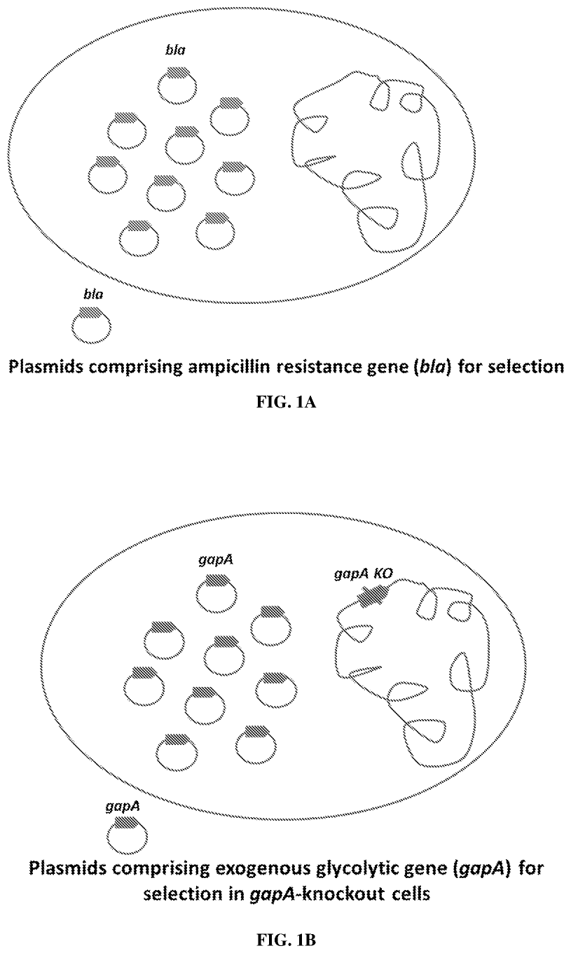

The glyceraldehyde 3-phosphate dehydrogenase (GAPDH) enzyme, encoded by the gapA gene in E. coli catalyzes the coupled oxidation and phosphorylation of glyceraldehyde-3-phosphate to 1,3-bisphosphoglycerate in the glycolytic pathway. Removal of this gene from E. coli effectively removes this chemical reaction to generate 1,3-bisphosphoglycerate and thus prevents growth in defined media or defined minimal media containing solely carbon sources that are upstream of GAPDH in glycolysis. Loss of gapA also prevents growth in complex media, which consist primarily of amino acids and small peptides, since gluconeogenesis would not be able to assimilate carbon past the gapA roadblock. To facilitate growth of gapA-deficient E. coli, such mutants need to be grown in media containing two or more carbon sources that can be assimilated into central metabolism on both sides of the gapA obstacle. One manifestation of such a medium is a medium containing both glycerol and succinic acid (e.g. sM63 media). To restore growth of gapA-deficient E. coli in a defined medium containing single carbon sources or a complex medium, a gene (e.g., gapA) coding for an enzyme having glyceraldehyde 3-phosphate dehydrogenase (GAPDH) activity (e.g., a reaction identified by EC 1.2.1.12 or 1.2.1.13) needs to be introduced into the cell on a plasmid or other DNA vector (FIG. 1B).

In some embodiments, an enzyme having GAPDH activity may come from E. coli or other organisms and have amino acid sequences given by accession numbers listed in Table 1 below. In some embodiments, an enzyme having GAPDH activity comprises or consists of an amino acid sequence belonging to any one enzyme described in Table 1. In some embodiments, an enzyme having GAPDH activity comprises or consists of an amino acid sequence having at least 75%, at least 80%, at least 85%, at least 90%, at least 95%, or at least 97% sequence identity to any one enzyme described in Table 1.

TABLE 1
Example Biological Sources of GAPDH Activity

| | Enzyme | Accession |
|---|---|---|
| Organism | Description | Number |
| unclassified | MULTISPECIES: type I glyceraldehyde- | WP_010874115 |
| Synechocystis | 3-phosphate dehydrogenase | |
| Bacillus subtilis | glyceraldehyde-3-phosphate | WP_172850264 |
| | dehydrogenase | |
| Escherichia fergusonii | glyceraldehyde-3-phosphate | EGC94929.1 |
| ECD227 | dehydrogenase A | |
| Synechocystis sp. | glyceraldehyde-3-phosphate | CAA58550.1 |
| PCC 6803 | dehydrogenase (NADP+) | |
| | (phosphorylating) | |
| Bacillus sp. | glyceraldehyde-3-phosphate | WP_069840105 |
| | dehydrogenase | |
| Salmonella enterica | glyceraldehyde-3-phosphate | EDV4562966.1 |
| subsp. enterica | dehydrogenase | |
| Synechocystis sp. | type I glyceraldehyde-3-phosphate | WP_028949102 |
| | dehydrogenase | |
| Bacillus halotolerans | glyceraldehyde-3-phosphate | WP_059293437 |
| | dehydrogenase | |
| Citrobacter freundii | glyceraldehyde-3-phosphate | GCB39387.1 |
| | dehydrogenase | |
| Bacillus mojavensis | glyceraldehyde-3-phosphate | WP_010335261 |
| | dehydrogenase | |
| Klebsiella oxytoca | glyceraldehyde-3-phosphate | WP_154680132 |
| | dehydrogenase | |
| Erwinia sp. | glyceraldehyde-3-phosphate | WP_154776494 |
| | dehydrogenase | |
| Bacillus atrophaeus | glyceraldehyde-3-phosphate | WP_106046853 |
| | dehydrogenase | |
| Enterobacter sp. | glyceraldehyde-3-phosphate | WP_072568225 |
| | dehydrogenase | |
| Serratia | MULTISPECIES: glyceraldehyde-3- | WP_061794510 |
| | phosphate dehydrogenase | |
| Buttiauxella sp. | glyceraldehyde-3-phosphate | WP_139877650 |
| | dehydrogenase | |
| Photorhabdus | glyceraldehyde-3-phosphate | WP_054479710 |
| heterorhabditis | dehydrogenase | |
| Pectobacterium fontis | glyceraldehyde-3-phosphate | WP_039344862.1 |
| | dehydrogenase | |
| Bacillus nakamurai | glyceraldehyde-3-phosphate | WP_061523064 |
| | dehydrogenase | |
| Proteus mirabilis | glyceraldehyde-3-phosphate | WP_049236155.1 |
| | dehydrogenase | |
| Erwinia sp. | glyceraldehyde-3-phosphate | WP_152601358 |
| | dehydrogenase | |
| Bacillus velezensis | glyceraldehyde-3-phosphate | WP_038459455 |
| | dehydrogenase | |
| Bacillus | MULTISPECIES: glyceraldehyde-3- | WP_046341391 |
| | phosphate dehydrogenase | |
| Microcystis sp. | type I glyceraldehyde-3-phosphate | TRT63741 |
| | dehydrogenase | |
| Microcystis aeruginosa | type I glyceraldehyde-3-phosphate | WP_045359498 |
| | dehydrogenase | |
| Bacillus pumilus | glyceraldehyde-3-phosphate | WP_099726907 |
| | dehydrogenase | |
| Crocosphaera subtropica | type I glyceraldehyde-3-phosphate | WP_009544928 |
| | dehydrogenase | |
| Bacillus altitudinis | glyceraldehyde-3-phosphate | WP_148943455 |
| | dehydrogenase | |
| Pleurocapsa minor | type I glyceraldehyde-3-phosphate | WP_015145817 |
| | dehydrogenase | |
| Cyanobacteria bacterium | TPA: type I glyceraldehyde-3-phosphate | HBQ98189.1 |
| UBA11691 | dehydrogenase | |
| Bacillus | glyceraldehyde-3-phosphate | WP_075751970 |
| paralicheniformis | dehydrogenase | |
| Bacillus licheniformis | glyceraldehyde-3-phosphate | WP_075646668 |
| | dehydrogenase | |
| Cyanobacteria bacterium | TPA: type I glyceraldehyde-3-phosphate | HAG79636.1 |
| UBA12227 | dehydrogenase | |
| Symploca sp. SIO3E6 | type I glyceraldehyde-3-phosphate | NES21715.1 |
| | dehydrogenase | |
| Bacillus swezeyi | glyceraldehyde-3-phosphate | WP_148958107 |
| | dehydrogenase | |
| Stanieria cyanosphaera | type I glyceraldehyde-3-phosphate | WP_015191528 |
| | dehydrogenase | |
| Leptolyngbya sp. | type I glyceraldehyde-3-phosphate | WP_015132368 |
| | dehydrogenase | |
| Aliterella atlantica | type I glyceraldehyde-3-phosphate | WP_045056004 |
| | dehydrogenase | |
| Bacillus aquimaris | glyceraldehyde-3-phosphate | WP_071619161 |
| | dehydrogenase | |
| Anoxybacillus | glyceraldehyde-3-phosphate | WP_004889084 |
| flavithermus | dehydrogenase | |
| Fischerella thermalis | type I glyceraldehyde-3-phosphate | WP_102152019 |
| | dehydrogenase | |
| Leptolyngbya sp. | type I glyceraldehyde-3-phosphate | WP_006515538 |
| | dehydrogenase | |
| Cytobacillus | glyceraldehyde-3-phosphate | WP_144543549 |
| oceanisediminis | dehydrogenase | |
| Synechococcales | type I glyceraldehyde-3-phosphate | NJM56430.1 |
| cyanobacterium | dehydrogenase | |
| RU_4_20 | | |
| Bacillus megaterium | glyceraldehyde-3-phosphate | WP_158314290 |
| | dehydrogenase | |
| Bacillus coagulans | glyceraldehyde-3-phosphate | WP_035184387 |
| | dehydrogenase | |
| Mesobacillus | glyceraldehyde-3-phosphate | WP_167832728 |
| selenatarsenatis | dehydrogenase | |
| Bacillus simplex | glyceraldehyde-3-phosphate | WP_137018190 |
| | dehydrogenase | |
| Halobacillus litoralis | glyceraldehyde-3-phosphate | WP_128526244 |
| | dehydrogenase | |
| Tetzosporium hominis | glyceraldehyde-3-phosphate | WP_094944121 |
| | dehydrogenase | |
| Alkalihalobacillus | glyceraldehyde-3-phosphate | WP_034749852 |
| wakoensis | dehydrogenase | |

In some embodiments, an enzyme having GAPDH activity comprises or consists of an amino acid sequence of SEQ ID NO: 50. In some embodiments, an enzyme having GAPDH activity comprises or consists of an amino acid sequence having at least 75%, at least 80%, at least 85%, at least 90%, at least 95%, or at least 97% sequence identity to an amino acid sequence of SEQ ID NO: 50.

GAPDH enzyme encoded by gapA gene in E. coli
(WP_000153502) -
SEQ ID NO: 50
MTIKVGINGFGRIGRIVFRAAQKRSDIEIVAINDLLDADYMAYMLKYDST
HGRFDGTVEVKDGHLIVNGKKIRVTAERDPANLKWDEVGVDVVAEATGLF
LTDETARKHITAGAKKVVMTGPSKDNTPMFVKGANFDKYAGQDIVSNASC
TTNCLAPLAKVINDNFGIIEGLMTTVHATTATQKTVDGPSHKDWRGGRGA
SQNIIPSSTGAAKAVGKVLPELNGKLTGMAFRVPTPNVSVVDLTVRLEKA
ATYEQIKAAVKAAAEGEMKGVLGYTEDDVVSTDFNGEVCTSVFDAKAGIA
LNDNFVKLVSWYDNETGYSNKVLDLIAHISK

### Genes Encoding an Outer Membrane Efflux Protein

Outer membrane efflux proteins are transmembrane protein channels that enable export of biological molecules (e.g., toxins) in gram-negative bacteria (e.g., E. coli). In some embodiments, an outer membrane efflux protein is a critical protein in promoting resistance to antibiotics in bacteria (e.g., pathogenic bacteria). In some embodiments, an outer membrane efflux protein is a critical protein in promoting survivability of bacteria (e.g., by enabling export of toxins from inside of a bacterium). In some embodiments, an outer membrane efflux pump is a protein selected from the group consisting of a tolC (e.g., tolC from E. coli), elongation factor G (FusA, e.g., FusA from Pseudomonas), mexA, mexB, oprM, PpF1, SepA, SepB, SepC, SmeC, OpmE, OpmD, OpmB, and bepC.

TolC is a protein efflux pump (e.g., the enzyme encoded by the tolC gene in E. coli). It serves as a channel through the periplasmic space and outer membrane of microbial cells (e.g., E. coli) to pump out (i.e., efflux) a variety of toxic compounds, including surfactant molecules such as sodium dodecyl sulfate (SDS). It plays an important role in conferring resistance to a broad spectrum of toxic exogenous compounds, such as antibiotics, detergents and organic solvents. TolC functions as a trimeric protein that is recruited to form a transient complex with translocases AcrAB or MdtABC, after such translocases have bound to a toxic compound. This interaction with AcrAB or MdtABC causes a conformational change in the structure of TolC to an open state, such that TolC can pump the toxic compound delivered by these efflux systems out of the cell. Importantly, loss of tolC renders microbial cells unable to grow in the presence of some of the toxic compounds that TolC is responsible for pumping out of the cell (e.g., surfactants such as SDS). Accordingly, the inventors of the present disclosure have found that plasmid addiction systems that utilize tolC are highly efficient. Deletion of endogenous tolC causes a microbial cell to require maintenance of a plasmid containing exogenous tolC if the microbial cell is being grown in the presence of surfactants such as SDS. In some embodiments, the TolC outer membrane channel protein may come from E. coli or homologs thereof in other organisms which have amino acid sequences given by accession numbers listed in Table 2 below.

TABLE 2
Example Biological Sources of TolC Activity.

| | Enzyme | Accession |
|---|---|---|
| Organism | Description | No. |
| Escherichia coli | Outer membrane protein TolC | ADD58252 |
| Escherichia sp. | outer membrane channel protein TolC | WP_131890779 |
| Shigella dysenteriae | outer membrane channel protein TolC | WP_128881184 |
| Enterobacteriaceae | MULTISPECIES: outer membrane | WP_097494326 |
| | channel protein TolC | |
| Shigella flexneri | outer membrane channel protein TolC | WP_095762548 |
| Shigella sonnei | outer membrane channel protein TolC | WP_094337337 |
| Shigella boydii | outer membrane channel protein TolC | WP_000735272 |
| Shigella sp. | outer membrane channel protein TolC | WP_069372140 |
| unclassified Escherichia | MULTISPECIES: outer membrane | WP_064529627 |
| | channel protein TolC | |
| Escherichia albertii | outer membrane channel protein TolC | WP_059268094 |
| Escherichia fergusonii | outer membrane channel protein TolC | WP_000735267 |
| Citrobacter farmeri | outer membrane channel protein TolC | WP_084196646 |
| Citrobacter amalonaticus | outer membrane channel protein TolC | WP_081093661 |
| Citrobacter youngae | outer membrane channel protein TolC | WP_032940764 |
| Salmonella enterica | outer membrane channel protein TolC | ECG8589487 |
| Citrobacter sp. | outer membrane channel protein TolC | WP_135324313 |
| Citrobacter koseri | outer membrane channel protein TolC | WP_130028136 |
| Citrobacter werkmanii | outer membrane channel protein TolC | WP_121529552 |
| Salmonella bongori | outer membrane channel protein TolC | WP_015703015 |
| Citrobacter portucalensis | outer membrane channel protein TolC | WP_003828407 |
| Citrobacter freundii | outer membrane channel protein TolC | WP_151217736 |
| Citrobacter rodentium | outer membrane channel protein TolC | WP_081442326 |
| unclassified Citrobacter | MULTISPECIES: outer membrane | WP_153752240 |
| | channel protein TolC | |

In some embodiments, a protein having tolC activity may come from E. coli or other organisms and have amino acid sequences given by accession numbers listed in Table 2. In some embodiments, a protein having tolC activity comprises or consists of an amino acid sequence belonging to any one protein described in Table 2. In some embodiments, a protein having tolC activity comprises or consists of an amino acid sequence having at least 75%, at least 80%, at least 85%, at least 90%, at least 95%, or at least 97% sequence identity to any one enzyme described in Table 2.

In some embodiments, a protein having tolC activity comprises or consists of an amino acid sequence of SEQ ID NO: 51. In some embodiments, a protein having tolC activity comprises or consists of an amino acid sequence having at least 75%, at least 80%, at least 85%, at least 90%, at least 95%, or at least 97% sequence identity to an amino acid sequence of SEQ ID NO: 51.

TolC enzyme encoded by tolC gene in E. coli
(ADD58252)
(SEQ ID NO: 51)
MIALNTASPQGMQMKKLLPILIGLSLSGFSSLSQAENLMQVYQQARLSNP
ELRKSAADRDAAFEKINEARSPLLPQLGLGADYTYSNGYRDANGINSNAT
SASLQLTQSIFDMSKWRALTLQEKAAGIQDVTYQTDQQTLILNTATAYFN
VLNAIDVLSYTQAQKEAIYRQLDQTTQRFNVGLVAITDVQNARAQYDTVL
ANEVTARNNLDNAVEQLRQITGNYYPELAALNVENFKTDKPQPVNALLKE
AEKRNLSLLQARLSQDLAREQIRQAQDGHLPTLDLTASTGISDTSYSGSK
TRGAAGTQYDDSNMGQNKVGLSFSLPIYQGGMVNSQVKQAQYNFVGASEQ
LESAHRSVVQTVRSSFNNINASISSINAYKQAVVSAQSSLDAMEAGYSVG
TRTIVDVLDATTTLYNAKQELANARYNYLINQLNIKSALGTLNEQDLLAL
NNALSKPVSTNPENVAPQTPEQNAIADGYAPDSPAPVVQQTSARTTTSNG
HNPFRN

### Cells and Genetic Modification

Cells (e.g., bacterial cells such as E. coli) of the disclosure may be modified (e.g., genetically modified, e.g., using a lambda red recombineering approach) to remove, inactivate or deplete one or more of its endogenous genes. In some embodiments, cells of the disclosure are modified to remove, inactivate or deplete one or more endogenous glycolytic genes (e.g. a prokaryotic gene encoding glyceraldehyde-3-phosphate dehydrogenase, such as the gapA gene from E. coli, or any protein as is described in Table 1) such that the cell lacks or has decreased expression of the endogenous glycolytic genes. In some embodiments, cells of the disclosure are modified to remove, inactivate or deplete one or more endogenous genes encoding an outer membrane efflux protein (e.g. a prokaryotic gene encoding an outer membrane efflux protein, such as the tolC gene from E. coli, or any protein as is described in Table 2) such that the cell lacks or has decreased expression of the endogenous gene encoding an outer membrane efflux protein. In some embodiments, a cell that has decreased expression of an endogenous gene expresses the gene at levels that are about 25%, 20%, 15%, 10%, 5%, 4%, 3%, 2%, 1%, or less than 1% of the expression level of a wild-type or unmodified cell. In some embodiments, the expression of an endogenous gene in a modified cell is at least 10%, 20%, 30%, 40%, 50%, 75%, 100%, 125%, 150%, 175%, or 200% less than the expression of the endogenous gene in a wild-type or unmodified cell. In some embodiments, cells are modified to be partially deficient in an endogenous gene (e.g., express low levels of the glycolytic gene). In some embodiments, a cell that is partially deficient in an endogenous gene expresses the gene at expression levels that are about 25%, 20%, 15%, 10%, 5%, 4%, 3%, 2%, 1%, or less than 1% of the expression level of a wild-type or native cell. In some embodiments, cells are genetically-modified to be wholly deficient in an endogenous gene (e.g., no detectable levels of the glycolytic gene). A cell that has been modified such that one or more endogenous genes (e.g., one or more endogenous glycolytic genes or an outer membrane protein) have been removed, inactivated or depleted may also be referred to as being deficient in said endogenous genes (e.g., deficient in said endogenous glycolytic genes or gene and encoding outer membrane protein).

In some embodiments, a cell that has been modified such that one or more endogenous glycolytic genes have been removed, inactivated or depleted cannot survive, propagate, or grow in a defined medium or defined minimal medium (e.g., Korz medium). In some embodiments, a cell that has been modified such that one or more endogenous glycolytic genes have been removed, inactivated or depleted cannot survive, propagate, or grow in a complex medium (e.g., Luria broth). In some embodiments, a cell that has been modified such that one or more endogenous glycolytic genes have been removed, inactivated or depleted cannot survive, propagate, or grow in a defined medium, defined minimal medium (e.g., Korz medium) and/or a complex medium (e.g., Luria broth).

In some embodiments, a cell that has been modified such that an endogenous glycolytic gene encoding an enzyme having glyceraldehyde-3-phosphate dehydrogenase (GAPDH) activity has been removed, inactivated or depleted. In some embodiments, a cell has been modified such that it lacks or has decreased expression of an endogenous glycolytic gene encoding an enzyme having glyceraldehyde-3-phosphate dehydrogenase (GAPDH) activity In some embodiments, an enzyme having GAPDH activity is a gapA gene from E. coli or a gene as described in Table 1.

In some embodiments, a cell that has been modified such that an endogenous glycolytic gene encoding an enzyme having GAPDH activity has been removed, inactivated or depleted cannot survive, propagate, or grow in a defined medium or defined minimal medium (e.g., Korz medium). In some embodiments, a cell that has been modified such that an endogenous glycolytic gene encoding an enzyme having GAPDH activity has been removed, inactivated or depleted cannot survive, propagate, or grow in a complex medium (e.g., Luria broth). In some embodiments, a cell that has been modified such that an endogenous glycolytic gene encoding an enzyme having GAPDH activity has been removed, inactivated or depleted cannot survive, propagate, or grow in a defined medium, defined minimal medium (e.g., Korz medium) or a complex medium (e.g., Luria broth).

In some embodiments, a cell that has been modified such that one or more endogenous genes encoding an outer membrane efflux protein have been removed, inactivated or depleted cannot survive, propagate, or grow in a medium comprising a surfactant (e.g., SDS). In some embodiments, a cell that has been modified such that one or more endogenous genes encoding an outer membrane efflux protein have been removed, inactivated or depleted cannot survive, propagate, or grow in a medium comprising a toxin.

In some embodiments, a cell that has been modified such that an endogenous gene encoding a protein having tolC activity has been removed, inactivated or depleted. In some embodiments, a cell has been modified such that it lacks or has decreased expression of an endogenous gene encoding a protein having tolC activity In some embodiments, a protein having tolC activity is a tolC protein from E. coli or a protein as described in Table 2.

In some embodiments, a cell that has been modified such that an endogenous gene encoding a protein having tolC activity has been removed, inactivated, or depleted cannot survive, propagate, or grow in a defined medium or defined minimal medium (e.g., Korz medium). In some embodiments, a cell that has been modified such that an endogenous gene encoding a protein having tolC activity has been removed, inactivated, or depleted cannot survive, propagate, or grow in a complex medium (e.g., Luria broth). In some embodiments, a cell that has been modified such that an endogenous gene encoding a protein having tolC activity has been removed, inactivated, or depleted cannot service, propagate, or grow in a defined medium, defined minimal medium (e.g., Korz medium) or a complex medium (e.g., Luria broth). Cells may be modified using any methods known to a skilled person. For example, cells may be modified using CRISPR/Cas9 technology, bacterial recombination, phage transduction (e.g. bacteriophage P1 transduction) and/or chromosomal deletions. In some embodiments, cells are modified using bacterial recombination that are performed using phage recombination proteins produced within the bacterial cells. In some embodiments, recombination refers to artificial joining of complementary nucleotide sequences of DNA from different organisms. Recombination proteins, such as single strand-annealing proteins or integrases, allow for the efficient introduction of sequences to or deletion of sequences from the bacterial genome. In some embodiments, recombination can be performed to replace an endogenous glycolytic gene (e.g. a prokaryotic gene encoding glyceraldehyde-3-phosphate dehydrogenase, such as the gapA gene from E. coli or a prokaryotic gene encoding tolC, such as the tolC gene from E. coli) with a non-coding sequence or a coding sequence that encodes an unrelated protein.

In some embodiments, the cell is a microbial cell from a unicellular or multicellular microorganism. In some embodiments, the cell is a bacterium. In some embodiments, the cell is a yeast cell. In some embodiments, a cell is a prokaryotic or eukaryotic cell. In some embodiments, a cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis) cell. In some embodiments, a cell is a Saccharomyces cerevisiae (S. cerevisiae), Yarrowia lypolytica (Y. lypolytica), Pichia pastoris (P. pastoris), or Trichoderma reesei (T. reesei) cell.

In some embodiments, the disclosure provides growing or culturing cells (e.g., in defined media or complex media). Growing or culturing cells describes the maintenance of cells in any growth media in order to promote cell survival and proliferation. In some embodiments, cells are grown in a defined minimal medium that consists of the minimal necessities for growth of said microbial cells. In some embodiments, minimal necessities consist of inorganic salts, a carbon source, a nitrogen source, and water. In some embodiments, a minimal medium is Korz broth, e.g., as defined in Korz et al., 1995, J. Biotechnol. 39:59-65. In some embodiments, a minimal medium is a modified Korz broth. In some embodiments, cells are grown in a complex medium that comprises inorganic salts, a carbon source, water, and at least one source of amino acids and nitrogen. In some embodiments, a complex medium is Luria Broth (LB).

An exogenous nucleic acid construct (e.g., a plasmid) comprising one or more exogenous genes may be added to a modified cell as described herein. In some embodiments, the exogenous genes are glycolytic genes (e.g., a gene encoding an enzyme having GAPDH activity). In some embodiments, the exogenous genes are genes encoding an outer membrane efflux protein (e.g., a gene encoding a protein having tolC activity). The exogenous nucleic acid construct may be added using electroporation, microinjection, bead transfection, calcium chloride transformation, or any transfection method known to a skilled person.

Addition of the exogenous nucleic acid construct (e.g., a plasmid) comprising one or more exogenous glycolytic genes (e.g., a gene encoding an enzyme having GAPDH activity) to the modified cell allows for the cell to survive, propagate, or grow in a defined minimal medium (e.g., Korz medium) and/or a complex medium (e.g., Luria broth). For example, addition of an exogenous nucleic acid construct comprising a gapA gene to a genetically modified gapA-deficient E. coli cell (ΔgapA E. coli cell) allows for the cell to survive, propagate, or grow in a defined minimal medium (e.g., Korz medium) and a complex medium (e.g., Luria broth).

Addition of the exogenous nucleic acid construct (e.g., a plasmid) comprising one or more exogenous genes encoding an outer membrane efflux protein (e.g., a gene encoding a protein having tolC activity) to the modified cell allows for the cell to survive, propagate, or grow in a medium comprising a toxin (e.g., a surfactant, including but not limited to SDS). For example, addition of an exogenous nucleic acid construct comprising a tolC gene to a genetically modified tolC-deficient E. coli cell (ΔtolC E. coli cell) allows for the cell to survive, propagate, or grow in a in a medium comprising a surfactant (e.g., SDS)

### Sequence of Interest and Encoded Products

The compositions of nucleic acids, e.g., DNA plasmids, described herein comprise a sequence of interest (SOI), wherein the SOI is any sequence that encodes an RNA, peptide and/or protein product. In some embodiments, an SOI is operably linked to a promoter, optionally a promoter comprising an initial transcription sequence (ITS). The promoter drives expression or drives transcription of the SOI that it regulates. In some embodiments, the sequence of interest is a gene of interest.

In some embodiments, an RNA product is a sense strand of a double-stranded RNA (dsRNA). In some embodiments, an RNA product is an antisense strand of a dsRNA. In some embodiments, a sense strand of a dsRNA is complementary to an antisense strand of a dsRNA. In some embodiments, an RNA product is a single-stranded RNA, e.g., messenger RNA. In some embodiments, an RNA product is shRNA, siRNA, an antisense oligonucleotide, a gapmer, or any other conceivable RNA product.

In some embodiments, an RNA product, e.g. a dsRNA, targets (e.g. via RNA interference) a genomic sequence of interest, e.g. from an insect, a plant, a fungus, an animal, or a virus. In some embodiments, an RNA product, e.g. an mRNA, encodes a protein of interest.

In some embodiments, an SOI that encodes an RNA product may have any length sufficient to induce biological activity. Non-limiting examples may include an SOI that encodes an RNA product with a length of 4 to 10, 4 to 20, 4 to 30, 4 to 50, 4 to 60, 4 to 70, 4 to 80, 4 to 90, 4 to 100, 4 to 200, 4 to 300, 4 to 400, 4 to 500, 4 to 1000, 4 to 2000, 4 to 3000, 4 to 4000, 4 to 5000, 4 to 6000, 4 to 7000, 4 to 8000, 4 to 9000 or 4 to 10000 nucleotides. In some embodiments, an SOI that encodes an RNA product has a length of 15, 16, 17, 18, 19, 20, 21, 22, 23, 24, or 25 nucleotides. In some embodiments, an SOI that encodes an RNA product has a length of 4, 5, 6, 7, 8, 9, 10, 15, 20, 25, 30, 35, 40, 45, 50, 55, 60, 65, 70, 75, 80, 85, 90, 95, 100, 500, 1000, or more nucleotides.

Two nucleic acids, e.g. the sense and antisense strands of dsRNA, are complementary to one another if they base-pair, or bind, to each other to form a double-stranded nucleic acid molecule via Watson-Crick interactions (also referred to as hybridization). As used herein, binding refers to an association between at least two molecules or two regions of the same molecule due to, for example, electrostatic, hydrophobic, ionic, and/or hydrogen-bond interactions under physiological conditions. In some embodiments, the two nucleic acids are 100% complementary. In some embodiments, the two nucleic acids are at least 75%, 80%, 85%, 90%, or 95% complementary.

In some embodiments, a double-stranded RNA or dsRNA is a wholly double-stranded molecule, which does not contain a single-stranded region (e.g., a loop or overhang). In some embodiments, a double-stranded RNA or dsRNA is a partially double-stranded molecule, which contains a double-stranded region and a single-stranded region (e.g. a loop or overhang).

In some embodiments, an SOI encodes an mRNA that allows synthesis of a peptide or protein product (e.g., an enzyme, antigen or antibody) upon translation. In some embodiments, an SOI encodes an mRNA that can be used as an mRNA vaccine. In some embodiments, a protein product is a kinase (e.g. CMP kinase, GMP kinase, UMP kinase, NDP kinase, polyphosphate kinase), phosphatase, epimerase, phosphoglucoisomerase (PGI), phosphoglucomutase (PGM), alpha-glucan phosphorylase, or isoamylase. In some embodiments, a protein product is a polymerase (e.g. T7 RNA Polymerase).

In some embodiments, an SOI that encodes a protein may have any length sufficient to enable large-scale production of the enzyme (e.g., CMP kinase, GMP kinase, UMP kinase, NDP kinase, polyphosphate kinase and T7 RNA polymerase). In some embodiments, an SOI that encodes a protein has a length of at least 100, 200, 300 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10,000 basepairs.

### Nucleic Acid Architecture

A nucleic acid described herein may comprise any conceivable architecture. In some embodiments, the nucleic acid is a circular plasmid. In some embodiments, the circular nucleic acid comprises an endonuclease recognition site that may allow for the circular nucleic acid to be linearized if the appropriate endonuclease cleaves the nucleic acid at said endonuclease recognition site. In some embodiments, the nucleic acid is a DNA template comprising a sequence of interest (SOI), wherein the SOI encodes an RNA product. In some embodiments, the DNA template or vector is a plasmid or a DNA construct. In some embodiments, the DNA template or vector is a plasmid, an expression cassette, a cosmid, a bacterial artificial chromosome, a yeast artificial chromosome, a bacteriophage, an adeno-associated viral vector (AAV vector), or a virus.

In some embodiments, the nucleic acid construct (e.g. a plasmid construct) comprises a replicon, defined as the minimal unit or element that allows replication of the nucleic acid construct (e.g. plasmid DNA) in the host microbial cell. In some embodiments, the replicon includes the origin of replication (ori) at which the replication of the nucleic acid construct (e.g. plasmid DNA) is initiated and additional elements that control the replication of the nucleic acid construct (e.g. a plasmid) and its copy number in the host cell. In embodiments where the nucleic acid construct is a plasmid DNA construct, the replication of plasmid DNA is initiated at the ori by the host's DNA replication machinery. Some non-limiting examples of replicons include those that allow replication of plasmids in bacterial hosts (e.g. E. coli) such as replicons found in the ColE1 plasmid, the pBR322 plasmid (pMB1 origin of replication), the pUC18 and pUC19 plasmids (carrying the pUC replicon, a derivative of the pMB1 replicon), the R6K plasmid, the p15A plasmid, the pSC101 plasmid etc. Different replicons result in different copy numbers and yields for plasmid in a given host. For example, the ColE1 and pMB1 origins typically allow maintenance of about 15-20 copies of plasmid molecules in each cell, while the deletion of the rop gene and two point mutations in the pMB1 origin result in the temperature-inducible amplification of copy number to 500-1000 copies per cell for plasmids carrying the pUC replicon as found in the pUC18- or pUC19-derived plasmids. Additionally, plasmids used in eukaryotic microbial cells (e.g. yeast) carry an ‘Autonomous Replication Sequence (ARS)’ as a replicon, where replication is initiated. In some embodiments, the replicon minimally consists of an origin of replication.

In some embodiments, a composition of a nucleic acid, e.g., a DNA plasmid, includes one or more promoters. A promoter may be naturally associated with a gene or sequence, e.g., an endogenous promoter. In some embodiments, an endogenous promoter is located upstream of the coding segment of a given gene or sequence. In some embodiments, a coding nucleic acid sequence, e.g. an SOI, may be positioned under the control of a recombinant or heterologous promoter, which refers to a promoter that is not normally associated with the encoded sequence in its natural environment. Such promoters may include promoters of other genes; promoters isolated from any other species; and synthetic promoters or enhancers that are not “naturally occurring” such as, for example, those that contain different elements of different transcriptional regulatory regions and/or mutations that alter expression through methods of genetic engineering that are known in the art. Non-limiting examples of promoters include: T7, T7Lac, SP6, P_{BAD}, Pₜᵣₚ, P_{lac}, Pₜᵣₐ, P_{trc}, lacUV5, T3, Pₜₑₜ, LuxR, OmpR, Pho A, Hsp-70 and Hsp-90 derived promoters, P_{cat}, Pₖₐₙ, P_{bla}, λpR, and λpL.

In some embodiments, the promoter is a constitutively active promoter. In some embodiments the promoter is an inducible promoter (e.g. a promoter that drives expression of an RNA transcript on induction with a chemical or a physical change). In some embodiments, the promoter is a chemically inducible promoter (e.g. a promoter such as the P_{BAD} promoter that is induced by addition of arabinose). In some embodiments a promoter is a temperature inducible promoter (e.g., a promoter that allows expression of a RNA transcript in response to a change in temperature). In some embodiments, the promoter is induced by the limitation of a nutrient during growth (e.g. the promoter that drives expression of the alkaline phosphatase gene phoA in E. coli in response to phosphate limitation). In some embodiments, the promoter is a promoter that naturally drives expression of the gene being utilized (e.g., the glycolytic gene being used or the outer membrane efflux protein being used). For example, in some embodiments, the promoter used to drive expression of a gapA gene from a plasmid is the natural gapA promoter found to drive the expression of these genes in the E. coli chromosome. As another example, in some embodiments, the promoter used to drive expression of a tolC gene from a plasmid is the natural tolC promoter found to drive the expression of these genes in the E. coli chromosome. In some embodiments, the promoter is a P_{bla} promoter or a P_{BAD} promoter. In some embodiments, the promoter is a T7 promoter (e.g., a T7 promoter comprising SEQ ID NO: 22). In some embodiments, the promoter comprises a T7 promoter sequence preceded by a 30 bp upstream sequence naturally present upstream of the class III T7 promoter that drives expression of the φ6.5, φ10 and φ13 genes in the T7 bacteriophage genome (e.g., a promoter comprising SEQ ID NO: 23). In some embodiments, the promoter is a synthetic promoter. In some embodiments, the synthetic promoter comprises an initial transcription sequence (ITS) comprising GGGAGACCGGGAATT (SEQ ID NO: 24). In some embodiments, the promoter comprises the nucleotide sequence of any one of SEQ ID NO: 1-23 or 52 in Table 3. In some embodiments, a promoter refers to the sequence of the non-template strand of a dsDNA segment placed upstream of a SOI encoding an RNA transcript to be expressed, with the transcription start site typically immediately downstream of the sequence. In some embodiments, when the promoter comprises a T7 promoter (e.g., a promoter comprising SEQ ID NO: 22 or 23), the transcription start site at the 5′ end of the RNA transcript to be expressed is a ‘G’ for efficient transcription.

TABLE 3
Promoter Sequences

| SEQ | | |
|---|---|---|
| ID | | |
| NO. | PROMOTER | SEQUENCE (5′ → 3′) |
| 1 | J23119 | TTGACAGCTAGCTCAGTCCTAGGTATAATGCTAGC |
| 2 | J23100 | TTGACGGCTAGCTCAGTCCTAGGTACAGTGCTAGC |
| 3 | J23101 | TTTACAGCTAGCTCAGTCCTAGGTATTATGCTAGC |
| 4 | J23102 | TTGACAGCTAGCTCAGTCCTAGGTACTGTGCTAGC |
| 5 | J23103 | CTGATAGCTAGCTCAGTCCTAGGGATTATGCTAGC |
| 6 | J23104 | TTGACAGCTAGCTCAGTCCTAGGTATTGTGCTAGC |
| 7 | J23105 | TTTACGGCTAGCTCAGTCCTAGGTACTATGCTAGC |
| 8 | J23106 | TTTACGGCTAGCTCAGTCCTAGGTATAGTGCTAGC |
| 9 | J23107 | TTTACGGCTAGCTCAGCCCTAGGTATTATGCTAGC |
| 10 | J23108 | CTGACAGCTAGCTCAGTCCTAGGTATAATGCTAGC |
| 11 | J23109 | TTTACAGCTAGCTCAGTCCTAGGGACTGTGCTAGC |
| 12 | J23110 | TTTACGGCTAGCTCAGTCCTAGGTACAATGCTAGC |
| 13 | J23111 | TTGACGGCTAGCTCAGTCCTAGGTATAGTGCTAGC |
| 14 | J23113 | CTGATGGCTAGCTCAGTCCTAGGGATTATGCTAGC |
| 15 | J23114 | TTTATGGCTAGCTCAGTCCTAGGTACAATGCTAGC |
| 16 | J23115 | TTTATAGCTAGCTCAGCCCTTGGTACAATGCTAGC |
| 17 | J23116 | TTGACAGCTAGCTCAGTCCTAGGGACTATGCTAGC |
| 18 | J23117 | TTGACAGCTAGCTCAGTCCTAGGGATTGTGCTAGC |
| 19 | J23118 | TTGACGGCTAGCTCAGTCCTAGGTATTGTGCTAGC |
| 20 | P_{bla} | CCTATTTGTTTATTTTTCTAAATACATTCAAATAT |
| | | GTATCCGCTCATGAGACAATAACCCT |
| 21 | P_{BAD} | ACTTTTCATACTCCCGCCATTCAGAGAAGAAACCA |
| | | ATTGTCCATATTGCATCAGACATTGCCGTCACTGC |
| | | GTCTTTTACTGGCTCTTCTCGCTAACCAAACCGGT |
| | | AACCCCGCTTATTAAAAGCATTCTGTAACAAAGCG |
| | | GGACCAAAGCCATGACAAAAACGCGTAACAAAAGT |
| | | GTCTATAATCACGGCAGAAAAGTCCACATTGATTA |
| | | TTTGCACGGCGTCACACTTTGCTATGCCATAGCAT |
| | | TTTTATCCATAAGATTAGCGGATCCTACCTGACGC |
| | | TTTTTATCGCAACTCTCTACTGTTTCTCCAT |
| 22 | T7 | TAATACGACTCACTATA |
| 23 | Extended- | TCGATTCGAACTTCTGATAGACTTCGAAATTAATA |
| | T7 | CGACTCACTATA |
| 52 | P_{tolC} | GGCACGTAACGCCAACCTTTTGCGGTAGCGGCTTC |
| | | TGCTAGAATCCGCAATAATTTTACA |

A ribosome binding site (RBS) is a segment of the 5′ untranslated region (5′ UTR) of an mRNA molecule to which the ribosome binds in order to position it correctly for initiating translation of an SOI encoding a peptide or protein at the start codon. The RBS controls the accuracy and efficiency with which the translation of mRNA begins and regulates protein synthesis based on its sequence and structure. There are two consensus sequences, Kozak and Shine-Dalgarno, which are known to facilitate efficient translation in eukaryotes and prokaryotes respectively. In eukaryotes, the Kozak consensus sequence is 5′-GCCACCAUGG-3′ (SEQ ID NO: 55). In prokaryotes, the Shine-Dalgarno consensus sequence is 5′-UAAGGAGG-3′ followed by an initiation codon, most commonly AUG. Activity of an RBS can be influenced by the length and nucleotide composition of the spacer separating the RBS and the initiator AUG. In some embodiments, an RBS is a natural RBS found as part of the 5′ UTR of an mRNA transcript expressed in an organism in nature. In some embodiments, an RBS is a synthetically designed construct, designed to achieve a desired translation initiation rate and protein production from a given mRNA transcript (e.g., RBS sequences as described in Kosuri et al, 2013, PNAS, 110:14024-14029 and Mutalik et al, 2012, Nature Methods, 10(4): 354-360). In some embodiments, an RBS as defined herein further comprises additional nucleotides of the 5′ UTR. In some embodiments, a ribosome binding site comprises the nucleotide sequence of any one of SEQ ID NO: 25-35 or 53.

TABLE 4
Ribosome Binding Site Sequences (may include
additional nucleotides of a 5′ UTR)

| SEQ | | |
|---|---|---|
| ID | | |
| NO. | RBS | SEQUENCE (5′ → 3′) |
| 25 | B0030 | AGATGATTAAAGAGGAGAAATTACAT |
| 26 | B0032 | AGATGTCACACAGGAAAGGCCCAT |
| 27 | B0033 | AGATGTCACACAGGACTTACAT |
| 28 | B0034 | AGATGAAAGAGGAGAAATTACAT |
| 29 | Salis-4-7 | AGATGAATCTCATATATCAAATATAAGCAGGATCAT |
| 30 | Salis-1-21 | AGATGAATCTCATATATCAAATATAGGGTGGATCAT |
| 31 | apFAB923 | AGATGATCTTAATCTAGCGTGGGAGAGTTTCAT |
| 32 | J61107 | AGATGTCTAGAGAAAGAAGAGACTCACCAT |
| 33 | bla_{rbs} | GATAAATGCTTCAATCATGATTGAAAAAGGAAGAGT |
| 34 | gapA_{rbs} | AACCTTTTATTCACTAACAAATAGCTGGTGGAATAT |
| 35 | araB_{rbs} | ACCCGTTTTTTTGGCTTTTGTGTAACTGTAAGAAGG |
| | | AGATATCAT |
| 53 | tolC | GTTTGATCGCGCTAAATACTGCTTCACCACAAGGA |

In some embodiments, a composition of a nucleic acid, e.g., a DNA plasmid, includes a transcriptional terminator sequence. The sequence encoding the transcriptional terminator is typically located immediately downstream of the coding sequence. It is comprised of a DNA sequence involved in specific termination of an RNA transcript by an RNA polymerase. A terminator sequence prevents transcriptional activation of downstream nucleic acid sequences by upstream promoters. Thus, in some embodiments, DNA templates comprising a terminator that ends the production of an RNA transcript are contemplated. The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strands. In some embodiments, reverse transcriptional terminators are provided, which usually terminate transcription on the reverse strand only. In prokaryotic systems, terminators usually fall into two categories: (1) rho-independent terminators, and (2) rho-dependent terminators. Rho-independent terminators are generally composed of palindromic sequences that form a stem loop rich in G-C base pairs followed by a string of uracil bases.

Terminators for use in accordance with the present disclosure include any terminator of transcription described herein or known to one of ordinary skill in the art. Non-limiting examples of terminators include the termination sequences of genes, such as, for example, the bovine growth hormone terminator, the E. coli ribosomal RNA T1T2 terminators, rrnBT1 and rrnBT2, the human preproparathyroid PTH terminator and viral termination sequences and their derivatives such as, for example, the TO terminator, the TE terminator, Lambda T1, T7, TT7, T7U, TT3 terminators, and other terminator sequences found and/or used in bacterial systems. In some embodiments, the termination signal may be a sequence that cannot be transcribed or translated, such as those resulting from a sequence truncation.

In some embodiments, a terminator comprises two or more individual and/or distinct terminator sequences or combinations thereof. In some embodiments, a terminator comprises a rrnBT1, rrnBT2, T7, TT7, T7U, TT3, and/or PTH terminator sequence. In some embodiments, a terminator is as described in Table 5.

TABLE 5
Terminator sequences

| SEQ ID | | | |
|---|---|---|---|
| NO. | TERM. ID | DESCRIPTION | SEQUENCE (5′ → 3′) |
| 36 | Term. 10 | T7 terminator | AACCCCTTGGGGCCTCTAAACGGGTCTTGA |
| | | | GGGGTTTTTTG |
| 37 | Term. 18 | Combination of the | CATCTGTTTTCTTGCAAGATCAGCTGAGCA |
| | | natural PTH and | ATAACTAGCATAACCCCTTGGGGCCTCTAA |
| | | pET-T7 terminators | ACGGGTCTTGAGGGGTTTTTTGCTGAAAGG |
| | | | AGGAACTATATCCGGA |
| 38 | Term. 26 | Combination of | CCTAGCATAACCCCGCGGGGCCTCTTCGGG |
| | | synthetic T7U | GGTCTCGCGGGGTTTTTTGCTGAAAGAAGC |
| | | terminator, rrnBT1 | TTCAAATAAAACGAAAGGCTCAGTCGAAA |
| | | and the pET-T7 | GACTGGGCCTTTCGTTTTATCTGTTGTTTGT |
| | | terminator | CGCTGCGGCCGCACTCGAGCACCACCACCA |
| | | | CCACCACTGAGATCCGGCTGCTAACAAAGC |
| | | | CCGAAAGGAAGCTGAGTTGGCTGCTGCCAC |
| | | | CGCTGAGCAATAACTAGCATAACCCCTTGG |
| | | | GGCCTCTAAACGGGTCTTGAGGGGTTTTTT |
| | | | GCTGAAAGGAGGAACTATATCCGGA |
| 39 | Term. 34 | Combination of the | CCTAGCATAAACCCCTTGGGTTCCCTCTTTA |
| | | T3 terminator, | GGAGTCTGAGGGGTTTTTTGCTGAAAGAAG |
| | | rrnBT1 and the | CTTCAAATAAAACGAAAGGCTCAGTCGAA |
| | | pET-T7 terminator | AGACTGGGCCTTTCGTTTTATCTGTTGTTTG |
| | | | TCGCTGCGGCCGCACTCGAGCACCACCACC |
| | | | ACCACCATTGAGATCCGGCTGCTAACAAAG |
| | | | CCCGAAAGGAAGCTGAGTTGGCTGCTGCCA |
| | | | CCGCTGAGCAATAACTAGCATAACCCCTTG |
| | | | GGGCCTCTAAACGGGTCTTGAGGGGTTTTT |
| | | | TGCTGAAAGGAGGAACTATATCCGGA |
| 40 | Term-Quad | Combination of | AAGCTTGCTTAAGCAGAAGGCCATCCTGAC |
| | | rrnBT2, TT7, PTH | GGATGGCCTTTTTGCGTTTCTACCTAGCATA |
| | | and TT7 | ACCCCTTGGGGCCTCTAAACGGGTCTTGAG |
| | | terminators | GGGTTTTTTGGCCATCTGTTTTCTTGCAAGA |
| | | | TCAGCTGAGCAATAACTAGCATAACCCCTT |
| | | | GGGGCCTCTAAACGGGTCTTGAGGGGTTTT |
| | | | TTG |
| 41 | rrnBT1 | rrnBT1 terminator | TCAAATAAAACGAAAGGCTCAGTCGAAAG |
| | | sequence | ACTGGGCCTTTCGTTTTATCTGTTGTTTGTC |
| | | | GCTGCGGCC |
| 42 | rrnBT2 | rrnBT2 terminator | TTAAGCAGAAGGCCATCCTGACGGATGGCC |
| | | sequence | TTTTTGCGTTTCTAC |
| 43 | TT7 | TT7 terminator | CTAGCATAACCCCTTGGGGCCTCTAAACGG |
| | | sequence | GTCTTGAGGGGTTTTTTG |
| 44 | pET-T7 | pET-T7 terminator | GCTGAGCAATAACTAGCATAACCCCTTGGG |
| | | sequence | GCCTCTAAACGGGTCTTGAGGGGTTTTTTG |
| | | | CTGAAAGGAGGAACTATATCCGGA |
| 45 | T7U | T7U terminator | CCTAGCATAACCCCGCGGGGCCTCTTCGGG |
| | | sequence | GGTCTCGCGGGGTTTTTTGCTGAAAGAAGC |
| | | | T |
| 46 | TT3 | TT3 terminator | CCTAGCATAAACCCCTTGGGTTCCCTCTTTA |
| | | sequence | GGAGTCTGAGGGGTTTTTTGCTGAAAGAAG |
| | | | CT |
| 47 | PTH | PTH terminator | CATCTGTTTT |
| | | sequence | |
| 48 | TgapA | gapA terminator | TGTGATCTAAAAAGAGCGACTTCGGTCGCT |
| | | sequence | CTTTTTTTTACCTGATAAAA |
| 49 | Tbla | bla terminator | CTGTCAGACCAAGTTTACTCATATATACTTT |
| | | | AGATTGATTTAAAACTTCATTTTTAATTTAA |
| | | | AAGGATCTAGGTGAAGATCCTTTTTGATAA |
| | | | TCTCATG |
| 54 | TtolC | tolC terminator | CTAGAGGCATCAAATAAAACGAAAGGC |
| | | sequence | TCAGTCGAAAGACTGGGCCTTTCGTTTT |
| | | | ATCTGTTGTTTGTCGGTGAACGCTCTCC |
| | | | TGAGTAGGACAAATCCGCCGCCCTAGA |

In some embodiments, a composition of a nucleic acid, e.g., a DNA plasmid, includes one or more multicloning sites (MCSs) or unique restriction endonuclease digestion sites. A multicloning site (MCS) or a polylinker region is a segment of a nucleic acid construct (e.g., a DNA plasmid) that contains multiple unique endonuclease restriction sites that allow inserting and ligating one or more desired nucleic acid sequences (e.g. expression cassettes, gene or interest, promoters etc.) that themselves carry the appropriate restriction endonuclease sites at their ends. Digestion of the sequence to be inserted and the nucleic acid construct carrying the MCS by the appropriate restriction enzymes generates the necessary sticky ends that facilitate interactions between the insert of the recipient nucleic acid construct. Ligation by an appropriate ligase in turn allows the necessary covalent bond formation to complete the insertion to form an intact nucleic acid molecule. Restriction endonuclease sites present in a nucleic acid construct independently or as part of an MCS may allow cleavage at the recognition site (e.g., sites recognized by conventional Type II restriction enzymes such as EcoRI, BamHI etc.) or at a defined distance from the recognition site (e.g. sites recognized by Type IIS restriction enzymes such as Esp3I or BspQI).

### Nucleic Acid Constructs Comprising an Essential Glycolytic Gene

In some embodiments, a nucleic acid construct for use in a plasmid addiction system described herein comprises a replicon (e.g., including an origin of replication and its control elements) and one or more expression cassettes for the recombinant expression of a glycolytic gene known to be essential for the survival and growth of a host bacterial cell that is deficient in the activity encoded by said glycolytic gene (e.g., said gene and other genes encoding enzymes with the essential glycolytic activity have been removed, inactivated or depleted from the host cell by genetic modification) and/or is incapable of expressing said glycolytic gene from its own chromosome. In some embodiments, the replicon of the nucleic acid construct is the ColE1 replicon or is one derived from pUC18 or pUC19 replicon. In some embodiments, the replicon is one derived from the pBR322, pUC, R6K, p15a or pSC101 replicon.

In some embodiments, the expression cassette(s) comprise a suitable promoter which drives the expression of the glycolytic gene, optionally operably linked to an initial transcription sequence (ITS) and/or a 5′UTR comprising a suitable ribosome binding site (RBS). In some embodiments, the expression cassette(s) comprise one or more terminators downstream of the glycolytic gene. The expression levels of the glycolytic gene from its expression cassette may be tuned using a combination of a natural or synthetic promoter and a natural or synthetic RBS placed upstream of the gene. For example, in some embodiments, the expression cassette comprises the Pbla promoter (SEQ ID NO. 20) or a promoter as described in Table 3, optionally in combination with an RBS as described in Table 4.

In some embodiments, the glycolytic gene is a gene encoding the enzyme glyceraldehyde-3-phosphate dehydrogenase (GAPDH) from a prokaryote (e.g., the gapA gene from E. coli). In some embodiments, the glycolytic gene is a gene encoding the enzyme glyceraldehyde-3-phosphate dehydrogenase (GAPDH) from a eukaryote. In some embodiments, the glycolytic gene is a gene encoding an enzyme having GAPDH activity. In some embodiments, the glycolytic gene encoding GAPDH is as described in Table 1.

In some embodiments, the nucleic acid construct capable of expressing the glycolytic gene further comprises one or more multicloning sites to facilitate the incorporation of additional nucleic acid constructs (e.g. expression cassettes for specific RNA transcripts, peptides or protein products).

In some embodiments, the nucleic acid construct further comprises one or more expression cassettes for the recombinant expression of RNA transcripts. The RNA transcripts may be products of interest (e.g. mRNA or dsRNA) or encode one or more protein products of interest (e.g., for expression in vitro or in vivo). The expression of the RNA transcript(s) for each sequence of interest in an expression cassette may be driven by a T7 promoter, a P_{BAD} promoter or any other promoters as described in Table 3.

### Nucleic Acid Constructs Comprising an Outer Membrane Efflux Protein

In some embodiments, a nucleic acid construct for use in a plasmid addiction system described herein comprises a replicon (e.g., including an origin of replication and its control elements) and one or more expression cassettes for the recombinant expression of an outer membrane efflux protein. In some embodiments, the replicon of the nucleic acid construct is the ColE1 replicon or is one derived from pUC18 or pUC19 replicon. In some embodiments, the replicon is one derived from the pBR322, pUC, R6K, p15a or pSC101 replicon.

In some embodiments, the expression cassette(s) comprise a suitable promoter which drives the expression of the gene encoding an outer membrane efflux protein, optionally operably linked to an initial transcription sequence (ITS) and/or a 5′UTR comprising a suitable ribosome binding site (RBS). In some embodiments, the expression cassette(s) comprise one or more terminators downstream of the gene encoding an outer membrane efflux protein. The expression levels of the gene encoding an outer membrane efflux protein from its expression cassette may be tuned using a combination of a natural or synthetic promoter and a natural or synthetic RBS placed upstream of the gene. For example, in some embodiments, the expression cassette comprises the Pbla promoter (SEQ ID NO. 20) or a promoter as described in Table 3, optionally in combination with an RBS as described in Table 4.

In some embodiments, the gene encoding an outer membrane efflux protein is a gene encoding a tolC protein from a prokaryote (e.g., the tolC gene from E. coli). In some embodiments, the gene encoding an outer membrane efflux protein is a gene encoding a tolC protein from a eukaryote. In some embodiments, the gene encoding an outer membrane efflux protein is a gene encoding a protein having tolC activity. In some embodiments, the gene encoding an outer membrane efflux protein is as described in Table 2.

In some embodiments, the nucleic acid construct capable of expressing the gene encoding an outer membrane efflux protein further comprises one or more multicloning sites to facilitate the incorporation of additional nucleic acid constructs (e.g., expression cassettes for specific RNA transcripts, peptides or protein products).

In some embodiments, the nucleic acid construct further comprises one or more expression cassettes for the recombinant expression of RNA transcripts. The RNA transcripts may be products of interest (e.g., mRNA or dsRNA) or encode one or more protein products of interest (e.g., for expression in vitro or in vivo). The expression of the RNA transcript(s) for each sequence of interest in an expression cassette may be driven by a T7 promoter, a P_{BAD} promoter or any other promoters as described in Table 3.

### Plasmid Addicted Cells

Some aspects of the disclosure describe microbial cells that are deficient in an essential glycolytic gene necessary for growth and survival of the cells (e.g., cells having a mutation or deletion or any modification that impairs the expression of the enzyme encoded by the gene). The microbial cells may be prokaryotic or eukaryotic cells. In some embodiments, the cells are deficient in a gene encoding the enzyme glyceraldehyde-3-phosphate dehydrogenase (GAPDH). In some embodiments, the microbial cells are E. coli cells and the deficient/impaired gene is gapA. In some embodiments, the microbial cells that are deficient in an essential glycolytic gene are incapable of growing in a defined medium, and/or defined minimal medium and/or a complex medium.

Other aspects of the disclosure describe microbial cells that are deficient in a gene encoding an outer membrane efflux protein necessary for growth and survival of the cells (e.g., cells having a mutation or deletion or any modification that impairs the expression of the protein encoded by the gene). The microbial cells may be prokaryotic or eukaryotic cells. In some embodiments, the cells are deficient in a gene encoding an outer membrane efflux protein (e.g., tolC). In some embodiments, the microbial cells are E. coli cells and the deficient/impaired gene is tolC. In some embodiments, the microbial cells that are deficient in a gene encoding an outer membrane efflux protein are incapable of growing in a medium containing a threshold level of a surfactant.

In some embodiments, the microbial cells further comprise a nucleic acid construct comprising an exogenous gene that restores the enzymatic function of the deficient/impaired gene (e.g., glycolytic gene or gene encoding an outer membrane efflux protein) in the cells. In some embodiments, the nucleic acid construct comprising an exogenous gene that restores the enzymatic function of the deficient/impaired glycolytic gene in the cells further restores the capability of the microbial cells to grow and survive in a defined medium and/or defined minimal medium (e.g., Korz medium) and/or complex medium (e.g., Luria Broth). In some embodiments, the nucleic acid construct comprising an exogenous gene that restores the enzymatic function of the deficient/impaired gene encoding an outer membrane efflux protein in the cells further restores the capability of the microbial cells to grow and survive in a medium containing a threshold level of a surfactant.

### Methods

The genetically-engineered microbial cells of the present disclosure (e.g., cells addicted to a gapA-expressing plasmid or cells addicted to a tolC-expressing plasmid) are capable of producing at least 0.1%, 0.25%, 0.5%, 0.6%, 0.7%, 0.8%, 0.9%, 1.0%, 1.25%, 1.5%, 1.75%, 2.0%, 2.5%, 3.0%, 3.5%, 4.0%, 4.5%, 5.0%, 5.5%, 6.0%, 6.5%, 7.5%, 8.0%, 8.5%, 9.0%, 9.5%, 10.0%, 12.5%, 15%, 20%, 25%, 30%, 35%, 40%, 45% or 50% g/g dry cell weight (DCW) of plasmid DNA. In some embodiments, the genetically-engineered microbial cells of the present disclosure (e.g., cells addicted to a gapA-expressing plasmid or cells addicted to a tolC-expressing plasmid) are capable of producing at least 0.1, 0.25, 0.5, 0.6, 0.7, 0.8, 0.9, 1.0, 1.25, 1.5, 1.75, 2.0, 2.5, 3.0, 3.5, 4.0, 4.5, 5.0, 7.5 or 10 grams of plasmid DNA per liter of fermentation volume. In some embodiments, the genetically-engineered microbial cells of the present disclosure (e.g., cells addicted to a gapA-expressing plasmid or cells addicted to a tolC-expressing plasmid) are capable of producing at least 10%, 20%, 30%, 40%, 50%, 60%, 70%, 80%, 90%, or 95% of the total plasmid DNA produced by a control microbial cell comprising an antibiotic resistance marker gene. In some embodiments, the genetically-engineered microbial cells of the present disclosure (e.g., cells addicted to a gapA-expressing plasmid or cells addicted to a tolC-expressing plasmid) are capable of producing at least 10%, 20%, 30%, 40%, 50%, 60%, 70%, 80%, 90%, or 95% more plasmid DNA than is produced by a control microbial cell comprising an antibiotic resistance marker gene.

The genetically-engineered microbial cells of the present disclosure (e.g., cells addicted to a gapA-expressing plasmid or cells addicted to a tolC-expressing plasmid) are capable of producing at least 0.1, 0.25, 0.5, 0.6, 0.7, 0.8, 0.9, 1.0, 1.25, 1.5, 1.75, 2.0, 2.5, 3.0, 3.5, 4.0, 4.5, 5.0, 7.5 or 10, 15, 20, 25, 30, 35, 40, 45 or 50 grams of RNA product (e.g., double-stranded RNA) per liter of fermentation volume. Alternatively, the genetically-engineered microbial cells of the present disclosure (e.g., cells addicted to a gapA-expressing plasmid or cells addicted to a tolC-expressing plasmid) are capable of producing at least 1%, 2.5%, 5%, 7.5%, 10%, 15%, 20%, 25%, 30%, 35%, 40%, 45% or 50% g/g dry cell weight (DCW) of RNA product (e.g., double-stranded RNA). In some embodiments, the genetically-engineered microbial cells of the present disclosure (e.g., cells addicted to a gapA plasmid or cells addicted to a tolC-expressing plasmid) are capable of producing at least 10%, 20%, 30%, 40%, 50%, 60%, 70%, 80%, 90%, or 95% of the total RNA product (e.g., double-stranded RNA) produced by a control microbial cell comprising an antibiotic resistance marker gene. In some embodiments, the genetically-engineered microbial cells of the present disclosure (e.g., cells addicted to a gapA-expressing plasmid or cells addicted to a tolC-expressing plasmid) are capable of producing at least 10%, 20%, 30%, 40%, 50%, 60%, 70%, 80%, 90%, or 95% more RNA product (e.g., double-stranded RNA) than is produced by a control microbial cell comprising an antibiotic resistance marker gene.

The genetically-engineered microbial cells of the present disclosure (e.g., cells addicted to a gapA plasmid or cells addicted to a tolC-expressing plasmid) are capable of producing at least 1, 2.5, 5, 6, 7, 8, 9, 10, 12.5, 15, 17.5, 20, 25, 30, 35, 40, 45, 50, 55 or 60 grams of a protein product of interest per liter of fermentation volume. Alternatively, the genetically-engineered microbial cells of the present disclosure (e.g., cells addicted to a gapA-expressing plasmid or cells addicted to a tolC-expressing plasmid) are capable of producing at least 1%, 2.5%, 5%, 7.5%, 10%, 15%, 20%, 25%, 30%, 35%, 40%, 45%, 50%, 55%, 60% g/g of DCW of protein product. In some embodiments, the genetically-engineered microbial cells of the present disclosure (e.g., cells addicted to a gapA-expressing plasmid or cells addicted to a tolC-expressing plasmid) are capable of producing at least 10%, 20%, 30%, 40%, 50%, 60%, 70%, 80%, 90%, 95%, or 100% of the total protein product produced by a control microbial cell comprising an antibiotic resistance marker gene. In some embodiments, the genetically-engineered microbial cells of the present disclosure (e.g., cells addicted to a gapA-expressing plasmid or cells addicted to a tolC-expressing plasmid) are capable of producing at least 10%, 20%, 30%, 40%, 50%, 60%, 70%, 80%, 90%, 95%, or 100% more protein product than is produced by a control microbial cell comprising an antibiotic resistance marker gene.

### Kits

Some aspects of the present disclosure provide kits. The kits may comprise, for example, an engineered nucleic acid or construct (e.g., a plasmid) as described herein and a plurality of microbial cells deficient in an endogenous glycolytic gene (e.g., endogenous gapA). In some embodiments, the plurality of microbial cells is lyophilized or frozen in a cryoprotectant.

The kits described herein may include one or more containers housing components and optionally, instructions of uses. Kits for research purposes may contain the components in appropriate concentrations or quantities for running various experiments. Any of the kits described herein may further comprise components needed for performing any methods described herein.

Each component of the kits, where applicable, may be provided in liquid form (e.g., in solution), or in solid form, (e.g., a dry powder). In certain cases, some of the components may be lyophilized, reconstituted, or processed (e.g., to an active form), for example, by the addition of a suitable solvent or other species (for example, water or certain organic solvents), which may or may not be provided with the kit.

In some embodiments, the kits include instructions and/or promotion for use of the components provided. Instructions can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc.

The kits may contain any one or more of the components described herein in one or more containers. The components may be prepared sterilely, packaged in syringe and shipped refrigerated. Alternatively, it may be housed in a vial or other container for storage. A second container may have other components prepared sterilely. Alternatively, the kits may include the active agents premixed and shipped in a vial, tube, or other container.

The kits may also include other components, depending on the specific application, for example, containers, cell media, salts, buffers, reagents, syringes, needles, disposable gloves, etc.

### OTHER EMBODIMENTS

The present disclosure further provides embodiments relating to plasmid addiction systems based on outer membrane efflux proteins that are encompassed by the following numbered paragraphs:

1. A microbial cell lacking or having decreased expression of an endogenous gene that encodes an outer membrane efflux protein, wherein the microbial cell comprises a nucleic acid construct comprising an expression cassette that encodes a recombinant outer membrane efflux protein and an expression cassette that encodes a sequence of interest, and wherein the sequence of interest is expressed when the microbial cell is grown in the presence of a threshold level of a surfactant.

2. The microbial cell of claim 1, wherein the recombinant outer membrane efflux protein has the same enzymatic activity as the endogenous gene that encodes an outer membrane efflux protein.

3. The microbial cell of claim 1 or 2, wherein the chromosomal DNA of the microbial cell comprises a genetic modification of the endogenous gene or an element controlling the expression of the endogenous gene that decreases the expression of the outer membrane efflux protein.

4. The microbial cell of claim 3, wherein the genetic modification is a mutation, insertion or deletion.

5. The microbial cell of any one of claims 1-4, wherein the nucleic acid construct is a plasmid, a vector, a cosmid, a bacterial artificial chromosome, a yeast artificial chromosome, a bacteriophage, a viral vector or any other.

6. The microbial cell of any one of claims 1-5, wherein the endogenous gene encodes a tolC, FusA, mexA, mexB, oprM, PpF1, SepA, SepB, SepC, SmeC, OpmE, OpmD, OpmB, or bepC protein.

7. The microbial cell of any one of claims 1-6, wherein the outer membrane efflux protein is a tolC, FusA, mexA, mexB, oprM, PpF1, SepA, SepB, SepC, SmeC, OpmE, OpmD, OpmB, or bepC protein.

8. The microbial cell of any one of claims 1-7, wherein the endogenous gene encodes a protein having tolC activity, and wherein the recombinant outer membrane efflux protein has tolC activity.

9. The microbial cell of any one of claims 1-8, wherein the endogenous gene encodes a tolC protein, and wherein the recombinant outer membrane efflux protein is a recombinant tolC protein.

10. The microbial cell of 9, wherein the recombinant tolC protein comprises an amino acid sequence of SEQ ID NO: 51.

11. The microbial cell of any one of claims 1-10, wherein the microbial cell is a prokaryotic or eukaryotic cell.

12. The microbial cell of any one of claims 1-11, wherein the microbial cell is a bacterial cell or a yeast cell.

13. The microbial cell of any one of claims 1-12, wherein the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell.

1. The microbial cell of any one of claims 1-13, wherein the microbial cell is an Escherichia coli (E. coli) cell, the endogenous gene is tolC, and the recombinant outer membrane efflux protein is a recombinant tolC protein.

14.2. The microbial cell of any one of claims 1-1, wherein the threshold level of the surfactant is a concentration of surfactant that halts cell growth and/or promotes cell death in a control microbial cell.

14.3. The microbial cell of claim 14.2, wherein the control microbial cell lacks or has decreased expression of an endogenous gene that encodes an outer membrane efflux protein and does not comprise a nucleic acid construct comprising an expression cassette that encodes a recombinant outer membrane efflux protein.

15. The microbial cell of any one of claims 1-14.3, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

16. The microbial cell of any one of claims 1-15, wherein the nucleic acid construct further comprises a replicon comprising an origin of replication and its control elements.

17. The microbial cell of claim 16, wherein the replicon is of bacterial origin.

18. The microbial cell of claim 16, wherein the replicon is the ColE1 replicon, the pUC replicon or is derived from the ColE1, pBR322, pUC, R6K, p15a or pSC101 replicon.

19. The microbial cell of any one of claims 1-18, wherein the expression cassette that encodes a recombinant outer membrane efflux protein comprises a promoter operably linked to the coding sequence for the recombinant outer membrane efflux protein.

20. The microbial cell of claim 19, wherein the promoter comprises a nucleic acid sequence set forth in any one of SEQ ID NO: 1-23 or 52.

21. The microbial cell of claim 19, wherein the promoter consists of a nucleic acid sequence set forth in any one of SEQ ID NO: 1-23 or 52.

22. The microbial cell of claim 19, wherein the expression cassette that encodes a recombinant outer membrane efflux protein further comprises an initial transcription sequence (ITS) upstream of the coding sequence for the recombinant outer membrane efflux protein.

23. The microbial cell of claim 22, wherein the ITS comprises a nucleic acid sequence set forth in SEQ ID NO: 24.

24. The microbial cell of claim 22, wherein the ITS consists of a nucleic acid sequence set forth in SEQ ID NO: 24.

25. The microbial cell of any one of claims 19-24, wherein the expression cassette that encodes a recombinant outer membrane efflux protein further comprises a 5′UTR comprising a ribosome binding site (RBS) placed upstream of the coding sequence for the recombinant outer membrane efflux protein and one or more terminators downstream of the coding sequence for the recombinant outer membrane efflux protein.

26. The microbial cell of claim 25, wherein the RBS comprises a nucleic acid sequence set forth in any one of SEQ ID NO: 25-35 or 53.

27. The microbial cell of claim 25, wherein the RBS consists of a nucleic acid sequence set forth in any one of SEQ ID NO: 25-35 or 53.

28. The microbial cell of any one of claims 25-27, wherein the one or more terminators comprises a nucleic acid sequence set forth in any one of SEQ ID NO: 36-49 or 54.

29. The microbial cell of any one of claims 25-27, wherein the one or more terminators consists of a nucleic acid sequence set forth in any one of SEQ ID NO: 36-49 or 54.

30. The microbial cell of any one of claims 1-29, wherein the sequence of interest encodes an RNA product, peptide product or protein product.

31. The microbial cell of claim 30, wherein the RNA product is a messenger RNA, an siRNA, a microRNA, a guide RNA, a sense strand of a double-stranded RNA, or an antisense strand of a double-stranded RNA.

32. The microbial cell of claim 30 or 31, wherein the nucleic acid construct comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

33. The microbial cell of any one of claims 30-32, wherein the expression cassette comprising a sequence of interest further comprises a promoter operably linked to the sequence of interest.

34. The microbial cell of claim 33, wherein the promoter comprises a nucleic acid sequence set forth in any one of SEQ ID NO: 1-23 or 52.

35. The microbial cell of claim 33, wherein the promoter consists of a nucleic acid sequence set forth in any one of SEQ ID NO: 1-23 or 52.

36. The microbial cell of any one of claims 30-35, wherein the expression cassette comprising a sequence of interest further comprises one or more of the sequence elements selected from the group consisting of: a promoter, an initial transcription sequence, a ribosome binding site, a restriction endonuclease site, and a terminator.

37. The microbial cell of any one of claims 1-36, wherein the microbial cell does not comprise an antibiotic resistance gene.

38. A plasmid addiction system comprising:
(i) a microbial cell comprising a genetic modification of a gene that encodes an outer membrane efflux protein, wherein the genetic modification reduces or abolishes the expression of the endogenous outer membrane efflux protein; and
(ii) a plasmid comprising an expression cassette that encodes a recombinant outer membrane efflux protein;
wherein the microbial cell cannot grow or propagate in a medium containing a threshold level of surfactant without incorporation of the plasmid.

39. The plasmid addiction system of claim 38, wherein the genetic modification comprises a mutation, insertion or deletion within the gene encoding an outer membrane efflux protein or a control element of the gene, optionally wherein the control element is a promoter or a ribosome binding site.

40. The plasmid addiction system of claim 38 or 39, wherein the recombinant outer membrane efflux protein has the same activity as the endogenous outer membrane efflux protein.

41. The plasmid addiction system of any one of claims 38-40, wherein the microbial cell can grow and propagate in a medium containing a surfactant if the plasmid is incorporated into the cell.

42. The plasmid addiction system of any one of claims 38-41, wherein the modified gene encodes a tolC, FusA, mexA, mexB, oprM, PpF1, SepA, SepB, SepC, SmeC, OpmE, OpmD, OpmB, or bepC protein.

43. The plasmid addiction system of any one of claims 38-42, wherein the recombinant outer membrane efflux protein is a tolC, FusA, mexA, mexB, oprM, PpF1, SepA, SepB, SepC, SmeC, OpmE, OpmD, OpmB, or bepC protein.

44. The plasmid addiction system of any one of claims 38-43, wherein the modified gene encodes an outer membrane efflux protein having tolC activity, and wherein the recombinant outer membrane efflux protein has tolC activity.

45. The plasmid addiction system of any one of claims 38-44, wherein the modified gene encodes a tolC protein, and wherein the recombinant outer membrane efflux protein is a tolC protein.

46. The plasmid addiction system of 45, wherein the tolC protein comprises an amino acid sequence of SEQ ID NO: 51.

47. The plasmid addiction system of any one of claims 38-46, wherein the microbial cell is a prokaryotic or eukaryotic cell, optionally wherein the microbial cell is a bacterial cell or a yeast cell.

48. The plasmid addiction system of any one of claims 38-46, wherein the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell.

49. The plasmid addiction system of any one of claims 38-48, wherein the microbial cell is an Escherichia coli (E. coli) cell, the inactivated gene is tolC, and the recombinant outer membrane efflux protein is a tolC protein.

50. The plasmid addiction system of any one of claims 38-49, wherein the threshold level of the surfactant is a concentration of surfactant that halts cell growth and/or promotes cell death in a control microbial cell.

50.1. The plasmid addiction system of claim 50, wherein the control microbial cell lacks or has decreased expression of an endogenous gene that encodes an outer membrane efflux protein and does not comprise a nucleic acid construct comprising an expression cassette that encodes a recombinant outer membrane efflux protein.

51. The plasmid addiction system of any one of claims 38-50.1, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

52. The plasmid addiction system of any one of claims 41-51, wherein the plasmid comprises a replicon comprising an origin of replication and its control elements that allows replication of the plasmid in the microbial cell, optionally wherein the replicon is the ColE1 replicon, the pUC replicon or is derived from the ColE1, pUC, pBR322, R6K, p15a or pSC101 replicon.

53. The plasmid addiction system of any one of claims 41-52, wherein the expression cassette that encodes a recombinant outer membrane efflux protein comprises a promoter operably linked to the coding sequence for the recombinant outer membrane efflux protein.

54. The plasmid addiction system of claim 53, wherein the promoter comprises a nucleic acid sequence set forth in any one of SEQ ID NO: 1-23 or 52.

55. The plasmid addiction system of claim 53, wherein the promoter consists of a nucleic acid sequence set forth in any one of SEQ ID NO: 1-23 or 52.

56. The plasmid addiction system of claims 53-55, wherein the expression cassette that encodes a recombinant outer membrane efflux protein further comprises an initial transcription sequence (ITS) upstream of the coding sequence for the recombinant outer membrane efflux protein.

57. The plasmid addiction system of claim 56, wherein the ITS comprises a nucleic acid sequence set forth in SEQ ID NO: 24.

58. The plasmid addiction system of claim 56, wherein the ITS consists of a nucleic acid sequence set forth in SEQ ID NO: 24.

59. The plasmid addiction system of any one of claims 53-58, wherein the expression cassette that encodes a recombinant outer membrane efflux protein further comprises a 5′UTR comprising a ribosome binding site (RBS) placed upstream of the coding sequence for the recombinant outer membrane efflux protein and one or more terminators downstream of the coding sequence for the recombinant outer membrane efflux protein.

60. The plasmid addiction system of claim 59, wherein the RBS comprises a nucleic acid sequence set forth in any one of SEQ ID NO: 25-35 or 53.

61. The plasmid addiction system of claim 59, wherein the RBS consists of a nucleic acid sequence set forth in any one of SEQ ID NO: 25-35 or 53.

62. The plasmid addiction system of any one of claims 59-61, wherein the one or more terminators comprises a nucleic acid sequence set forth in any one of SEQ ID NO: 36-49 or 54.

63. The plasmid addiction system of any one of claims 59-61, wherein the one or more terminators consists of a nucleic acid sequence set forth in any one of SEQ ID NO: 36-49 or 54.

64. The plasmid addiction system of any one of claims 59-63, wherein the plasmid further comprises an expression cassette comprising a sequence of interest, wherein the sequence of interest encodes an RNA product, peptide product or protein product.

65. The plasmid addiction system of claim 64, wherein the RNA product is a messenger RNA, an siRNA, a microRNA, a guide RNA, a sense strand of a double-stranded RNA, or an antisense strand of a double-stranded RNA.

66. The plasmid addiction system of claim 64 or 65, wherein the plasmid comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

67. The plasmid addiction system of any one of claims 64-66, wherein the expression cassette(s) comprising a sequence of interest further comprise a promoter operably linked to the sequence of interest.

68. The plasmid addiction system of claim 67, wherein the promoter comprises a nucleic acid sequence set forth in any one of SEQ ID NO: 1-23 or 52.

69. The plasmid addiction system of claim 67, wherein the promoter consists of a nucleic acid sequence set forth in any one of SEQ ID NO: 1-23 or 52.

70. The plasmid addiction system of any one of claims 64-69, wherein the expression cassette comprising a sequence of interest further comprises one or more of the sequence elements selected from the group consisting of: a promoter, an initial transcription sequence, a ribosome binding site, a restriction endonuclease site, and a terminator.

71. The plasmid addiction system of any one of claims 38-63, wherein the plasmid further comprises one or more multicloning sites (MCSs) or unique restriction endonuclease digestion sites.

72. The plasmid addiction system of any one of claims 38-71, wherein the plasmid does not comprise an antibiotic resistance gene.

73. A nucleic acid construct comprising an expression cassette comprising a gene encoding a protein having tolC activity and
(i) one or more multiple cloning sites, and/or
(ii) an expression cassette comprising a sequence of interest encoding an RNA product, peptide product or protein product.

74. The nucleic acid construct of claim 73, wherein the nucleic acid construct is a plasmid, a vector, a cosmid, a bacterial artificial chromosome, a yeast artificial chromosome, a bacteriophage, a viral vector or any other.

75. The nucleic acid construct of claim 73 or 74, wherein the gene encoding a protein having tolC activity is a microbial tolC gene.

76. The nucleic acid construct of any one of claims 73-75, wherein the protein having tolC activity comprises an amino acid sequence of SEQ ID NO: 51.

77. The nucleic acid construct of any one of claims 73-76, wherein the nucleic acid construct comprises a first sequence of interest and a second sequence of interest, optionally wherein a first expression cassette comprises the first sequence of interest and a second expression cassette comprises the second sequence of interest.

78. The nucleic acid construct of claim 77, wherein the first sequence of interest encodes a sense strand of a double-stranded RNA product, and the second sequence of interest encodes an antisense strand of a double-stranded RNA product.

79. The nucleic acid construct of any one of claims 73-78, wherein any one of the expression cassettes further comprises a promoter and/or terminator.

80. The nucleic acid construct of claim 79, wherein the promoter comprises a nucleic acid sequence set forth in any one of SEQ ID NO: 1-23 or 52.

81. The nucleic acid construct of claim 79, wherein the promoter consists of a nucleic acid sequence set forth in any one of SEQ ID NO: 1-23 or 52.

82. The nucleic acid construct of any one of claims 79-81, wherein the promoter is operably linked to an initial transcription sequence (ITS).

83. The nucleic acid construct of claim 82, wherein the ITS comprises a nucleic acid sequence set forth in SEQ ID NO: 24.

84. The nucleic acid construct of claim 82, wherein the ITS consists of a nucleic acid sequence set forth in SEQ ID NO: 24.

85. The nucleic acid construct of any one of claims 79-81, wherein the promoter is operably linked to a 5′UTR comprising a ribosome binding site (RBS).

86. The nucleic acid construct of claim 85, wherein the RBS comprises a nucleic acid sequence set forth in SEQ ID NO: 25-35 or 53.

87. The nucleic acid construct of claim 85, wherein the RBS consists of a nucleic acid sequence set forth in SEQ ID NO: 25-35 or 53.

88. A method comprising culturing the microbial cell of any one of claims 1-37 in the presence of a threshold level of a surfactant and the absence of an antibiotic under conditions sufficient to produce the nucleic acid construct.

89. The method of claim 88, wherein the method produces at least 50% of the total amount of the nucleic acid construct as produced by a control microbial cell comprising an antibiotic resistance marker gene.

90. The method of claim 88, wherein the method produces at least 90% of the total amount of the nucleic acid construct as produced by a control microbial cell comprising an antibiotic resistance marker gene.

91. A method comprising culturing the microbial cell of any one of claims 16-37 in the in the presence of a threshold level of a surfactant and the absence of an antibiotic under conditions sufficient to produce the RNA product, peptide product or protein product.

92. The method of claim 91, wherein the method produces at least 50% of the total amount of the RNA product, peptide product or protein product as produced by a control microbial cell comprising an antibiotic resistance marker gene.

93. The method of claim 91 or 92, wherein the method produces at least 90% of the total amount of the RNA product, peptide product or protein product as produced by a control microbial cell comprising an antibiotic resistance marker gene.

94. A method comprising:
delivering to a microbial cell a vector comprising a gene encoding tolC and a gene expressing a sequence of interest,
   wherein the microbial cell comprises a genetically modified tolC gene, optionally wherein the genetic modification comprises a mutation, insertion or deletion within the tolC gene or a control element of the tolC gene, further optionally wherein the control element is a promoter or a ribosome binding site.

95. The method of any one of claims 88-93, wherein the threshold level of the surfactant is a concentration of surfactant that halts cell growth and/or promotes cell death in a control microbial cell.

96. The method of claim 95, wherein the control microbial cell lacks or has decreased expression of an endogenous gene that encodes an outer membrane efflux protein and does not comprise a nucleic acid construct comprising an expression cassette that encodes a recombinant outer membrane efflux protein.

97. The method of any one of claims 88-93, 95, or 96, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

98. A kit comprising:
(i) the nucleic acid construct of any one of claims 73-87; and
(ii) a plurality of microbial cells comprising a genetically modified tolC gene, optionally wherein the genetic modification comprises a mutation, insertion or deletion.

99. A kit comprising:
(i) a plasmid comprising an expression cassette that encodes an outer membrane efflux protein; and
(ii) a plurality of microbial cells comprising a genetic modification of a gene that encodes an outer membrane efflux protein, optionally wherein the genetic modification comprises a mutation, insertion or deletion within the gene or a control element of the gene, further optionally wherein the control element is a promoter or a ribosome binding site.

100. A kit comprising a plurality of the microbial cell of any one of claims 1-37.

101. The kit of any one of claims 98-100, wherein the plurality are lyophilized or frozen in a cryoprotectant.

### EXAMPLES

### Example 1: Construction of ARMed Plasmids or unARMed Plasmids Employing a gapA-Based Selection

One set of plasmids utilizing gapA-based addiction for plasmid retention in bacterial hosts were constructed to demonstrate that such unARMed plasmids can be used to achieve levels of recombinant protein overexpression that are comparable to plasmids utilizing traditional antibiotic resistance markers (ARMed plasmids) for selection. Additionally, a second set of plasmids utilizing gapA-based addiction for plasmid retention were constructed to demonstrate that such unARMed plasmids can be used to obtain yields of template plasmid DNA comparable to those obtained with ARMed plasmids from E. coli fermentations. Details on the construction of these two sets of plasmids are given below. Additionally ARMed plasmids were constructed for use as controls.

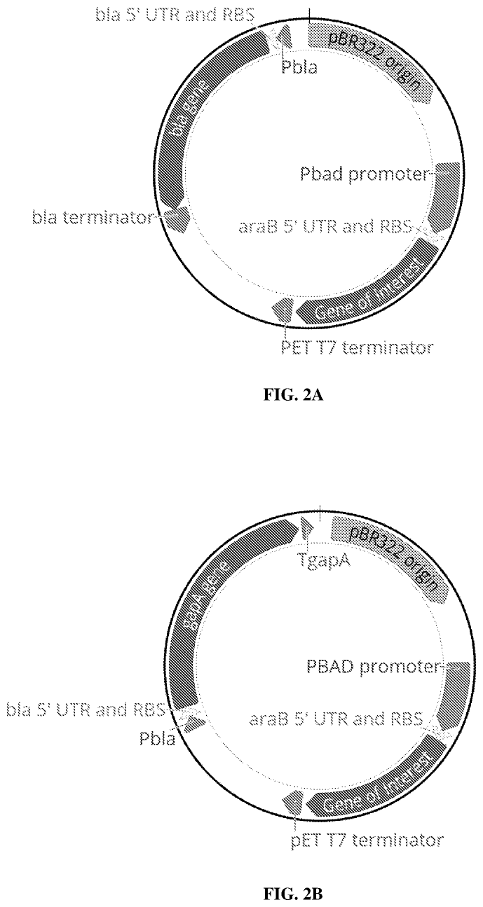

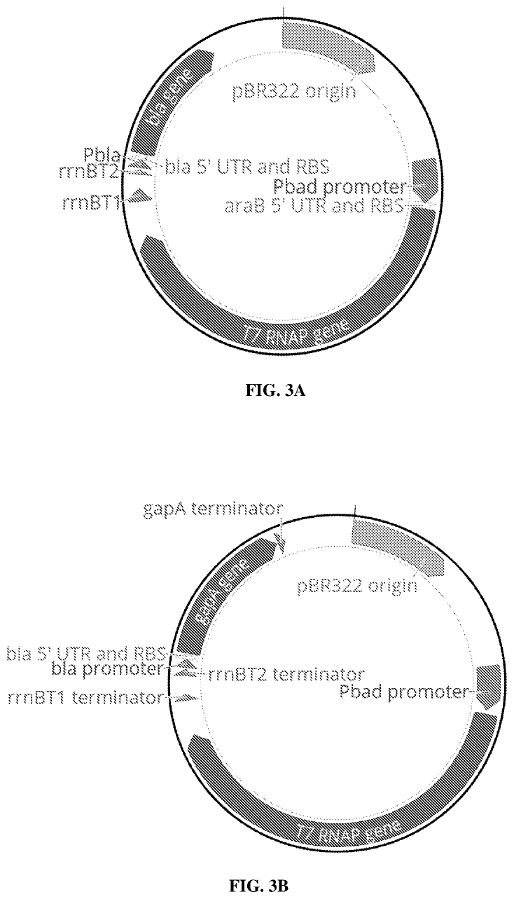

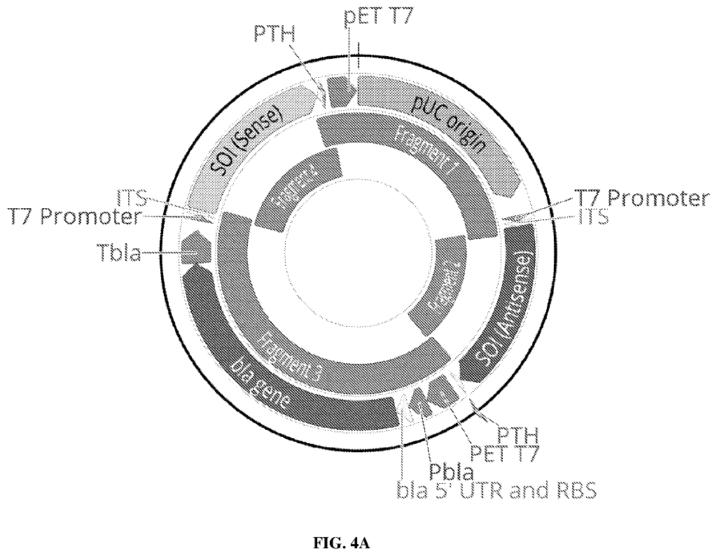

The ARMed expression plasmids described in Table 6 comprise a) an expression cassette for the expression of the bla gene (encoding β-lactamase) as an ampicillin-resistance marker under the transcriptional and translational control of the Pbla promoter (SEQ ID NO: 20) and the 5′UTR comprising the bla_{rbs} RBS(SEQ ID NO: 33) respectively with the Tbla terminator (Tbla, SEQ ID NO: 49) downstream of the bla gene, b) a pBR322 origin of replication and c) an expression cassette for the expression of recombinant proteins comprising a P_{BAD} promoter (SEQ ID NO: 21) and a 5′UTR comprising the araB_{rbs} RBS (SEQ ID NO: 35) placed upstream of two multi-cloning sites for up to two genes of interest or sequences of interest with a single pET-T7 terminator (SEQ ID NO: 44) downstream of these multi-cloning sites. An example ARMed plasmid template is shown in FIG. 2A.

The unARMed plasmids based on a gapA plasmid addiction system comprised a) an expression cassette for the E. coli gapA gene as an antibiotic-free marker, comprising the Pbla promoter (SEQ ID NO: 20) and the 5′UTR comprising the bla_{rbs} RBS (SEQ ID NO: 33) driving expression of the gapA gene with the TgapA terminator (SEQ ID NO: 48) downstream of the gapA gene, b) a pBR322 origin of replication, c) an expression cassette for the expression of recombinant enzymes comprising a P_{BAD} promoter (SEQ ID NO: 21) and a 5′UTR comprising the araB_{rbs} RBS (SEQ ID NO: 35) placed upstream of two multi-cloning sites for up to two genes of interest or sequences of interest with a single pET-T7 terminator (SEQ ID NO: 44) downstream of these multi-cloning sites. An example unARMed plasmid template is shown in FIG. 2B.

TABLE 6
ARMed and unARMed plasmids expressing recombinant proteins.

| | Selection | Recombinant |
|---|---|---|
| Plasmid | Marker | Protein |
| pGLA590 | bla gene (ARMed) | Enterobacteria phage T7 |
| | | RNA polymerase |
| pGLA678 | bla gene (ARMed) | Aquifex aeolicus |
| | | NDP kinase |
| pGLA680 | bla gene (ARMed) | Thermus thermophilus |
| | | CMP kinase |
| pGLA682 | bla gene (ARMed) | Pyrococcus furiosus |
| | | UMP kinase |
| pGLA684 | bla gene (ARMed) | Thermotoga maritima |
| | | GMP kinase |
| pGLA705 | bla gene (ARMed) | Deinococcus geothermalis |
| | | PP kinase |
| pGLX010 | gapA gene (unARMed) | Thermus thermophilus |
| | | CMP kinase |
| pGLX027 | gapA gene (unARMed) | Enterobacteria phage T7 |
| | | RNA polymerase |
| pGLX029 | gapA gene (unARMed) | Aquifex aeolicus |
| | | NDP kinase |
| pGLX031 | gapA gene (unARMed) | Pyrococcus furiosus |
| | | UMP kinase |
| pGLX033 | gapA gene (unARMed) | Thermotoga maritima |
| | | GMP kinase |
| pGLX035 | gapA gene (unARMed) | Deinococcus geothermalis |
| | | PP kinase |

Plasmids containing expression cassettes may themselves be the desired products of microbial fermentation and need to have origins of replication that support high-copy-number replication in order to maximize plasmid DNA (pDNA) yield. To demonstrate that such high-copy plasmids can be effectively produced using gapA addiction as the plasmid retention mechanism, several plasmids were constructed that contain the pUC origin of replication and transcription cassettes that enable synthesis of a dsRNA product (GS1).

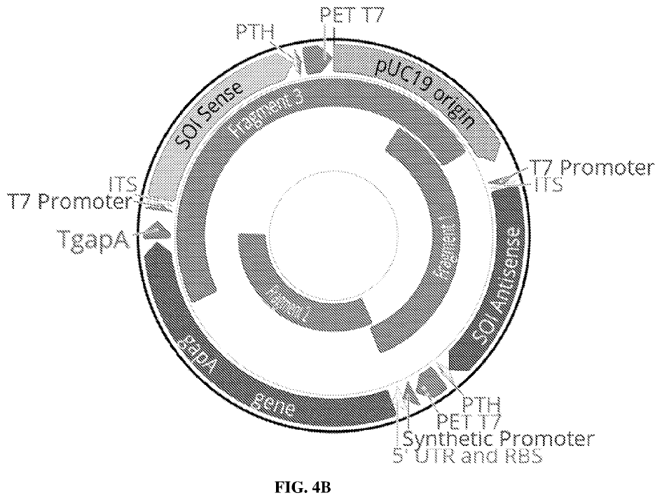

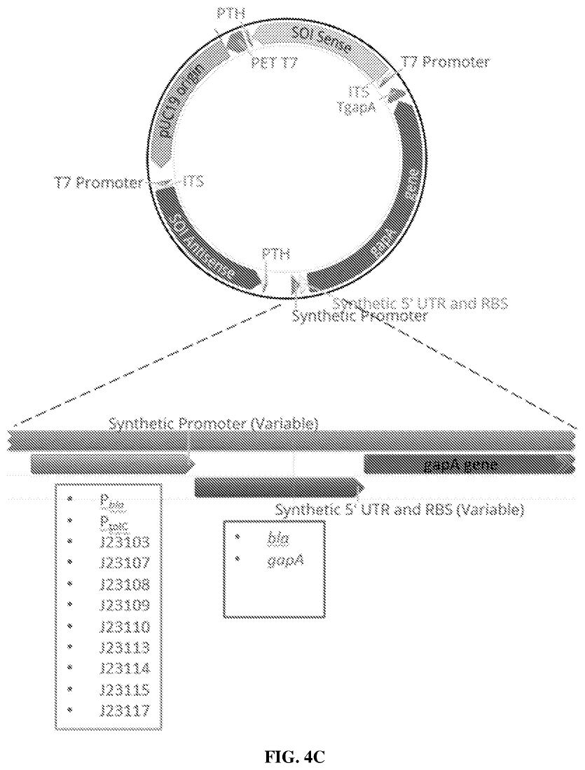

ARMed template plasmids containing the bla gene have a structure, represented by the plasmid map shown in FIG. 4A, consisting of two transcription cassettes, wherein each cassette, a sequence of interest to be transcribed is flanked by the Extended T7 promoter (SEQ ID NO: 23) and ITS (SEQ ID NO: 24) on the 5′ end and the Term 18 dual terminator complex (SEQ ID NO: 37) consisting of the PTH (SEQ ID NO: 47) and pET-T7 (SEQ ID NO: 44) terminators on the 3′ end. The pUC origin of replication is located between the 3′ end of the sense cassette and the 5′ end of the antisense cassette and expression of the bla gene was driven from an expression cassette comprising the Pbla promoter (SEQ ID NO: 20) and the 5′UTR comprising the bla_{rbs} RBS (SEQ ID NO: 33) upstream of the bla gene and the Tbla terminator downstream of it.

UnARMed template plasmids have a structure, as shown in FIG. 4B, that is similar to ARMed template plasmids except for the replacement of the bla gene with the E. coli gapA gene. A variety of promoter and RBSes as described in Table 7 were utilized to vary the level of expression of the gapA gene.

Table 7. ARMed and unARMed Template Plasmids for dsRNA Production

TABLE 7
ARMed and unARMed Template Plasmids for dsRNA Production

| | | Selection | Selection | |
|---|---|---|---|---|
| | | Marker | Marker | dsRNA |
| Plasmid | Selection Marker | Promoter | RBS | Product |
| pGLA709 | bla gene (ARMed) | Pbla | bla | GS1 |
| pGLX014 | gapA gene (unARMed) | Pbla | bla | GS1 |
| pGLX056 | gapA gene (unARMed) | J23108 | gapA | GS1 |
| pGLX057 | gapA gene (unARMed) | J23109 | gapA | GS1 |
| pGLX058 | gapA gene (unARMed) | J23110 | gapA | GS1 |
| pGLX059 | gapA gene (unARMed) | J23113 | gapA | GS1 |
| pGLX060 | gapA gene (unARMed) | J23114 | gapA | GS1 |
| pGLX061 | gapA gene (unARMed) | J23115 | gapA | GS1 |
| pGLX063 | gapA gene (unARMed) | J23117 | gapA | GS1 |
| pGLX098 | gapA gene (unARMed) | J23107 | B0032 | GS1 |

### Example 2: Development of a gapA-Based Plasmid Addiction System

The endogenous gapA gene was removed from the chromosome of E. coli cells (GL17-086, a BL21(DE3) derived strain that has the endogenous tolC gene deleted) using lambda red recombination. The endogenous gapA gene was replaced with a tolC gene marker that can be selected for by growth in the presence of 50 mg/L sodium dodecyl sulfate (SDS). Cells that acquired the desired ΔgapA::tolC replacement were confirmed by PCR amplification and sequencing of the corresponding locus of the genome.

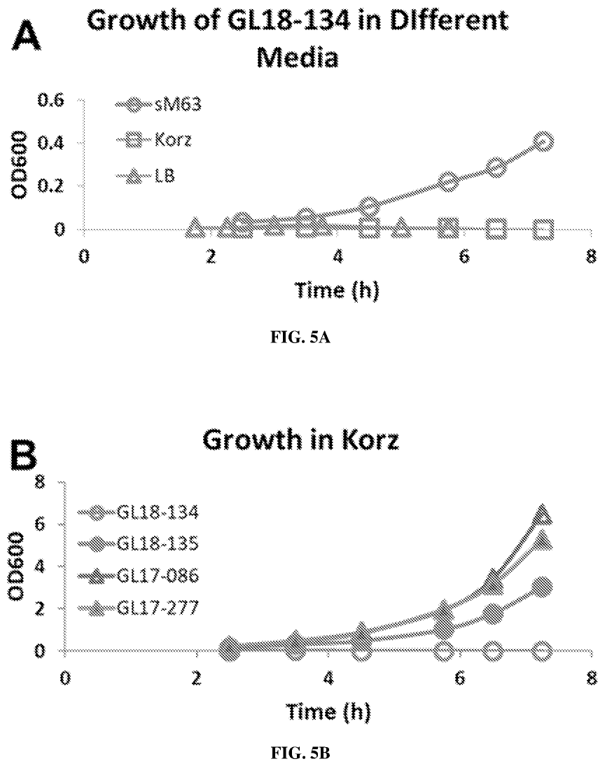

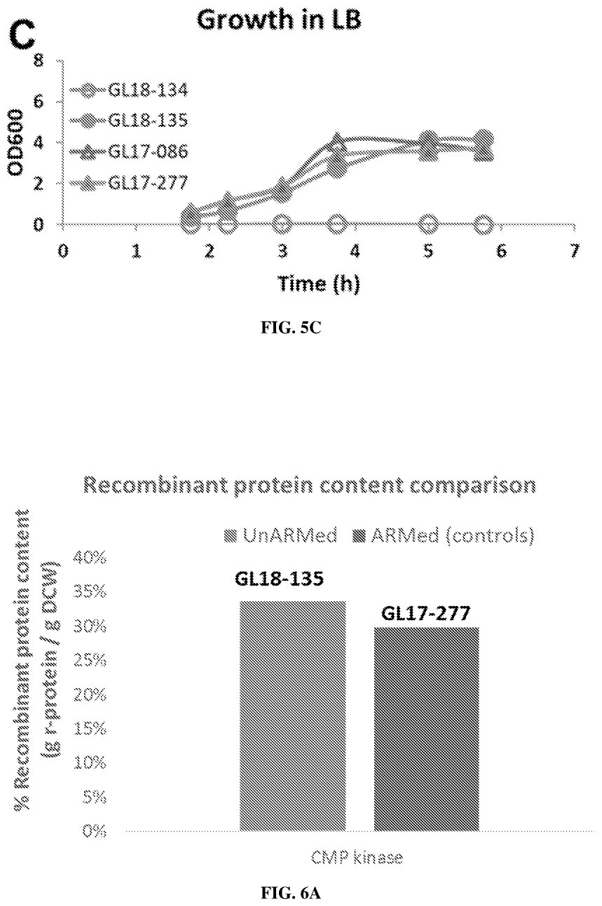

An isolated single colony of E. coli that was confirmed to contain the ΔgapA::tolC chromosomal modification was saved as strain GL18-134 (Table 8) and characterized for its ability to grow in a defined minimal medium containing glucose as the sole carbon source (Korz broth), Luria Broth (LB) medium, and an sM63 defined medium (See, Pardee et al., 1959, J. Mol. Biol. 1:165-178) containing 1 g/L casamino acids, 25 mM succinate and 12.5 mM glycerol. For comparison, strain GL17-086, the parent E. coli strain that is the gapA⁺ counterpart to GL18-134 was also grown in Korz broth and LB medium as a positive control (FIGS. 5A-5B).

TABLE 8
Strains described in Example 2

| | Baseline | | | Relevant |
|---|---|---|---|---|
| | Chromosome | | Plasmid | Plasmid |
| Strain | Genotype | Plasmid | Selection | Genotype |
| GL17-086 | BL21(DE3) | None | N/A | N/A |
| GL18-134 | BL21(DE3) | None | N/A | N/A |
| | ΔgapA::tolC | | | |
| GL17-277 | BL21(DE3) | pGLA680 | bla gene (ampicillin | pMB1-ori P_{BAD}-His- |
| | | | resistance) | Tthcmk-T_{T7} P_{bla}-bla-T_{bla} |
| GL18-135 | BL21(DE3) | pGLX010 | gapA gene | pMB1-ori P_{BAD}-His- |
| | ΔgapA::tolC | | | Tthcmk-T_{T7} P_{bla}-gapA- |
| | | | | T_{gapA} |

GL18-134 (ΔgapA, i.e., gapA-deficient) and GL17-086 (gapA⁺) strains were grown overnight in sM63 and Korz minimal media, respectively, at 37° C. with 250 rpm shaking. The next day, three sets of shake flasks, each containing either 10 mL of LB or Korz or sM63 media were inoculated with either 100 μL of GL18-134 or GL17-086 overnight culture and then incubated at 37° C. with 250 rpm shaking. Samples were taken periodically for OD₆₀₀ measurement.

FIGS. 5A-5B show the growth of GL18-134 (ΔgapA) and GL17-086 (gapA⁺) in either Korz, LB or sM63 media. GL18-134 was unable to grow in either Korz or LB (FIG. 5A), since these media contain carbon sources that require glycolysis or gluconeogenesis, respectively, to synthesize all the building blocks necessary for bacterial cells to remain viable. Growth was observed however in sM63 since this medium provides carbon sources (glycerol and succinate) that generate carbon fluxes on either side of gapA in the glycolytic pathway. Conversely, as anticipated, GL17-086 (gapA⁺) was able to grow in Korz and LB (FIG. 5B), to an optical density at 600 nm (OD₆₀₀) between 2.0-4.0, following 4-6 hours after inoculation.

Introduction of a plasmid that expresses gapA into the ΔgapA E. coli strain (GL18-135) allowed for restoration of growth (FIGS. 5B-5C). Specifically, transformation of ΔgapA E. coli with a pET-derived plasmid designated pGLX010, which carries the E. coli gapA gene with its natural 5′UTR (gapA_{rbs}, SEQ ID NO: 34) and terminator (TgapA, SEQ ID NO: 48) cloned to allow expression of gapA driven by the constitutive bla promoter, enables the resulting transformant (strain GL18-135, solid circles) to grow in both LB and Korz media. GL18-135 additionally expressed a recombinant protein (Thermus thermophilus CMP kinase) from a P_{BAD} promoter upon induction with L-arabinose. GL17-277, a transformant of GL17-086 carrying a bla-expressing plasmid pGLA680 was grown as a control for comparison in the presence of 50 mg/L carbenicillin in both Korz and LB media.

As shown in Table 9, a comparison of the doubling times for strain GL17-277 and GL18-135 shows that the use of gapA addiction to maintain the plasmid results in growth kinetics that are similar to those using a bla resistance marker and carbenicillin for plasmid maintenance. GL18-134, which is the gapA-deficient strain without a plasmid, can only grow in sM63 defined medium at a doubling time that is nearly 80% longer than when the strain is grown in Korz minimal medium with gapA plasmid rescue (GL18-135), thus demonstrating the effectiveness of complementing the ΔgapA phenotype with a gapA expressing plasmid.

TABLE 9
Growth parameters of a gapA-plasmid addicted strain and a strain
using antibiotic (carbenicillin) resistance for plasmid selection
in shake flask cultures incubated at 37° C. and 250 rpm shaking.

| | | Specific Growth | | | | | |
|---|---|---|---|---|---|---|---|
| | Plasmid | Rate (h⁻¹) | | | Doubling Time (min) | | |
| Strain | Selection | Korz | LB | sM63 | Korz | LB | sM63 |
| GL17-086 | N/A | 0.67 | 0.93 | ND | 44.9 | 61.6 | ND |
| (gapA⁺) | | | | | | | |
| GL18-134 | N/A | 0 | 0 | 0.54 | 0 | 0 | 76.6 |
| (ΔgapA) | | | | | | | |
| GL17-277 | carbenicillin | 0.66 | 0.82 | ND | 50.6 | 62.8 | ND |
| (gapA⁺) | resistance | | | | | | |
| | (pGLA680) | | | | | | |
| GL18-135 | gapA | 0.61 | 0.98 | ND | 42.6 | 68.4 | ND |
| (ΔgapA) | addiction | | | | | | |
| | (pGLX010) | | | | | | |

*ND, not determined

### Example 3: Production of a Recombinant Protein Using a gapA-Based Plasmid Addiction System in Fed-Batch Fermentations

GL18-135 (ΔgapA; addicted to gapA plasmid) and GL17-277 (gapA⁺, carbenicillin-resistant) strains, as developed in Example 2, were used for studying expression of recombinant proteins. Specifically, GL18-135 and GL17-277 were used to express Thermus thermophilus CMP kinase (CmpK) from gapA-based selection and carbenicillin resistance-based selection plasmids, respectively in fermentations. The seed train for the fermentations comprised of two precultures. During the first preculture stage, 25 mL of Luria-Broth (LB) medium in a culture flask was inoculated with 0.625 mL (2.5% v/v inoculum) of frozen cells (frozen stocks at an optical density (OD₆₀₀) of ˜1) and incubated in a rotary shaker at 37° C. and 300 rpm. After the cells reached an OD₆₀₀ of 2, 2.5 mL of this culture (2.5% v/v inoculum) was transferred to the second preculture stage flask containing 97.5 mL of defined medium (Korz medium), carrying 25 g/L of glucose as a carbon source. The second preculture flask was incubated in a rotary shaker at 37° C. and 300 rpm. When the cells reached an OD₆₀₀ of 3, 80 mL of this culture was transferred to a 2.4 L nominal volume (NV) bioreactor containing 720 mL of defined medium (10% v/v inoculum) with 25 g/L of glucose as carbon source. Carbenicillin was added to all cultures of strain GL17-277 at a concentration of 50 mg/L in the fermentation medium to maintain antibiotic selection pressure during growth. Growth in the presence of glucose as sole carbon source served as selection for plasmid carrying cells in the ΔgapA GL18-135 strain.

The fermentation process was comprised of a batch phase followed by a fed-batch fermentation phase. Both phases were controlled at a pH of 7.05, an airflow of 1 volume of air per volume of media (VVM), dissolved oxygen at or above 30%, and a temperature of 37° C. The pH was controlled with only 30% (v/v) ammonium hydroxide as base with no acid control. Dissolved oxygen was maintained at >30% using agitation and oxygen supplementation when needed. At the end of batch phase, glucose feed was initiated on a continuous basis, progressively ramping the feed rate in a linear mode. Once the OD₆₀₀ of the culture reached 75, L-arabinose was supplied at a constant rate until the end of the fermentation to express CMP kinase. The fermentation was conducted in a defined medium (Korz medium) containing salts (such as phosphate and sulfates), trace metals (such as magnesium, iron, calcium, manganese, and zinc) and vitamins (such as thiamine).

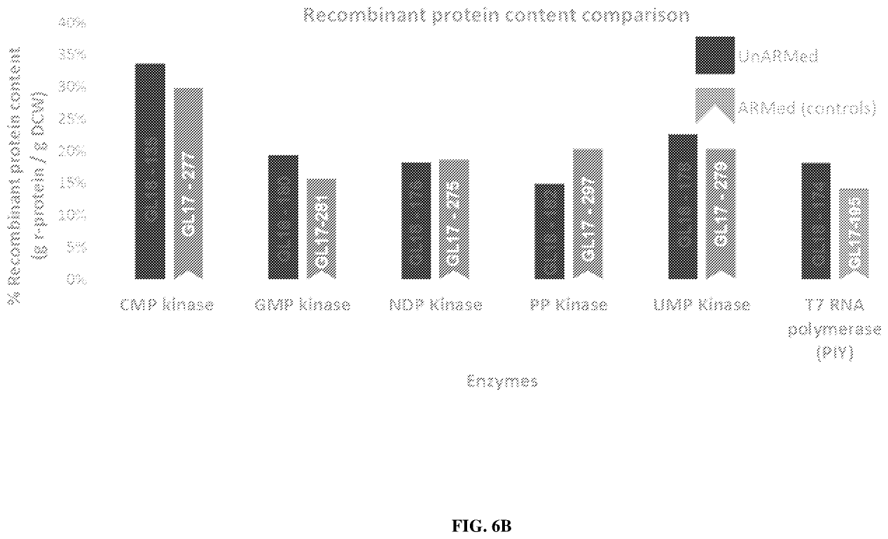

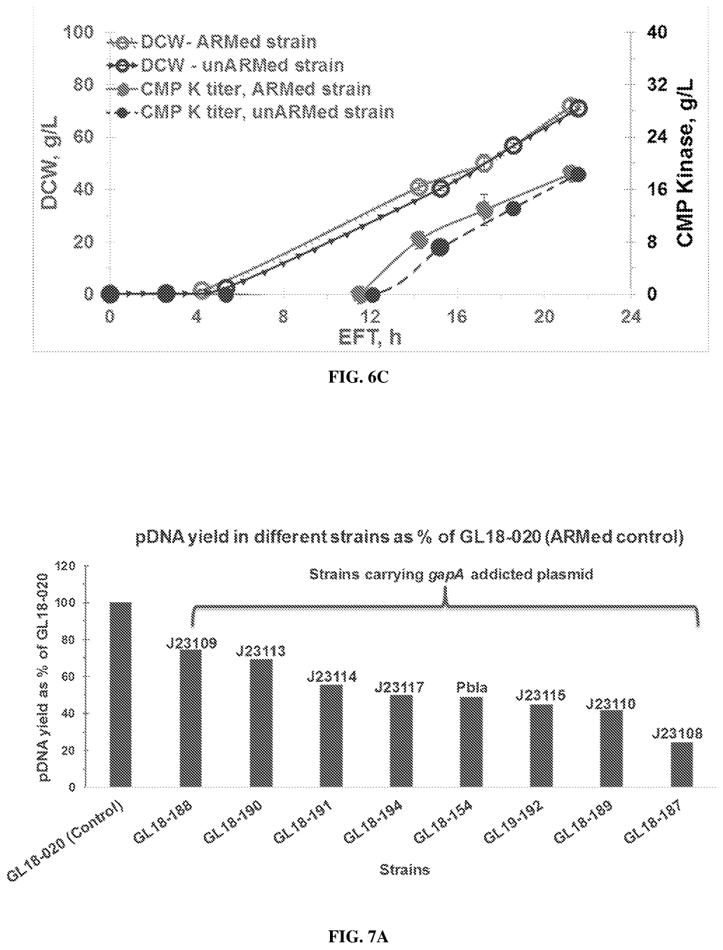

As shown in FIGS. 6A-6C, the GL18-135 strain (ΔgapA, addicted to gapA-containing plasmid pGLX010) produced levels of CMP kinase that were comparable to levels of enzyme produced by GL17-277 strain (gapA⁺, carbenicillin resistant). For example, the GL18-135 strain produced ˜20 g/L CMP kinase and a dry cell weight (DCW) of ˜70 g/L. Samples were collected at regular intervals to measure optical density and enzyme expression. Expression of the His-tagged enzymes were determined quantitatively by separating enzyme from the total expressed protein using SDS-polyacrylamide gel electrophoresis and selectively staining with a His-tag In-gel stain. ImageJ software was used to quantitatively compare the stained band intensities of enzymes between samples and purified enzyme standards run on the same gel.

### Example 4: Production of Recombinant Proteins Using a gapA-Based Plasmid Addiction System in Batch Fermentations in Shake-Flasks

The gapA addiction strategy described in Example 3 for expression of CMP kinase was similarly used to maintain plasmids engineered to express other recombinant proteins, such as GMP kinase, NDP kinase, PP kinase, UMP kinase, and T7 RNA polymerase in ΔgapA strains. As shown in FIG. 6B, ΔgapA strains produced recombinant protein in shake flasks at levels comparable to those expressed in gapA⁺ strains carrying expression plasmids with the carbenicillin-resistance marker and grown in media supplemented with 50 mg/L carbenicillin. The ARMed and unARMed strains used for this study are described in Table 10.

TABLE 10
Strains described in Examples 3-4

| Strain | Plasmid | Selection Marker | Recombinant Protein Produced |
|---|---|---|---|
| GL17-275 | pGLA678 | bla gene (ARMed) | Aquifex aeolicus |
| | | | NDP kinase |
| GL17-279 | pGLA682 | bla gene (ARMed) | Pyrococcus furiosus |
| | | | UMP kinase |
| GL17-281 | pGLA684 | bla gene (ARMed) | Thermotoga maritima |
| | | | GMP kinase |
| GL18-195 | pGLA590 | bla gene (ARMed) | Enterobacteria phage |
| | | | T7 RNA polymerase |
| GL17-297 | pGLA705 | bla gene (ARMed) | Deinococcus geothermalis |
| | | | PP kinase |
| GL17-277 | pGLA680 | bla gene (ARMed) | Thermus thermophilus |
| | | | CMP kinase |
| GL18-135 | pGLX010 | gapA gene (unARMed) | Thermus thermophilus |
| | | | CMP kinase |
| GL18-178 | pGLX031 | gapA gene (unARMed) | Pyrococcus furiosus |
| | | | UMP kinase |
| GL18-176 | pGLX029 | gapA gene (unARMed) | Aquifex aeolicus |
| | | | NDP kinase |
| GL18-174 | pGLX027 | gapA gene (unARMed) | Enterobacteria phage |
| | | | T7 RNA polymerase |
| GL18-182 | pGLX035 | gapA gene (unARMed) | Deinococcus geothermalis |
| | | | PP kinase |
| GL18-180 | pGLX033 | gapA gene (unARMed) | Thermotoga maritima |
| | | | GMP kinase |

### Example 5: Production of Plasmid DNA Using a gapA-Based Plasmid Addiction System

Production of plasmid DNA as a desired product via fermentation at a high yield and titer requires the stable maintenance and propagation of high copy plasmids in microbial hosts. The use of the gapA plasmid addiction system to maintain a high-copy plasmid with a pUC origin of replication in E. coli was comparable to the maintenance of a similar high-copy pUC plasmid employing a bla antibiotic resistance marker for plasmid maintenance. The pUC origin of replication allows temperature-inducible amplification of plasmid copy number.

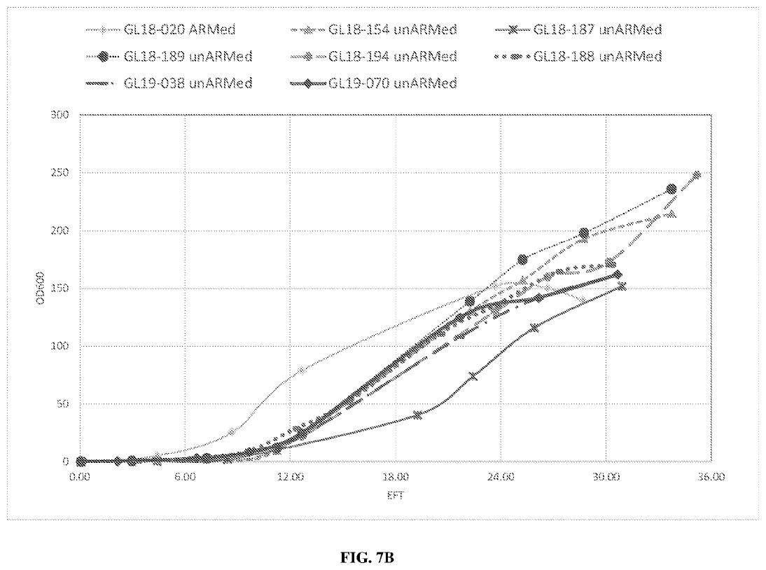

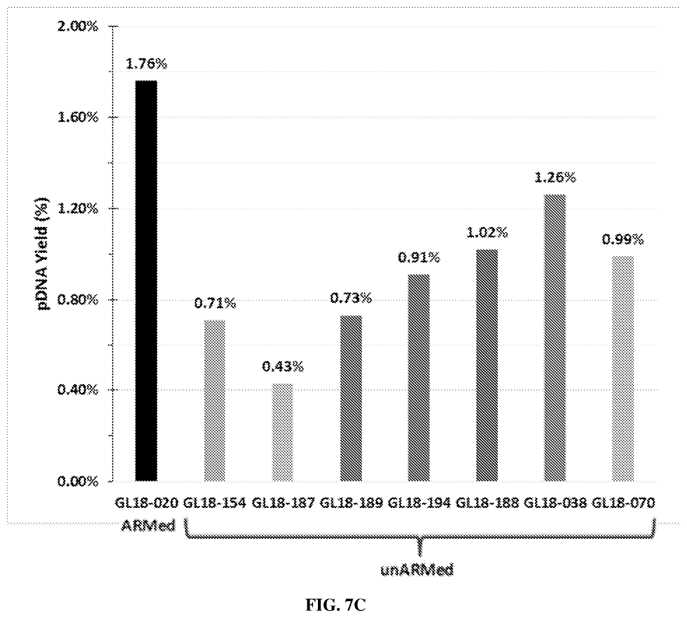

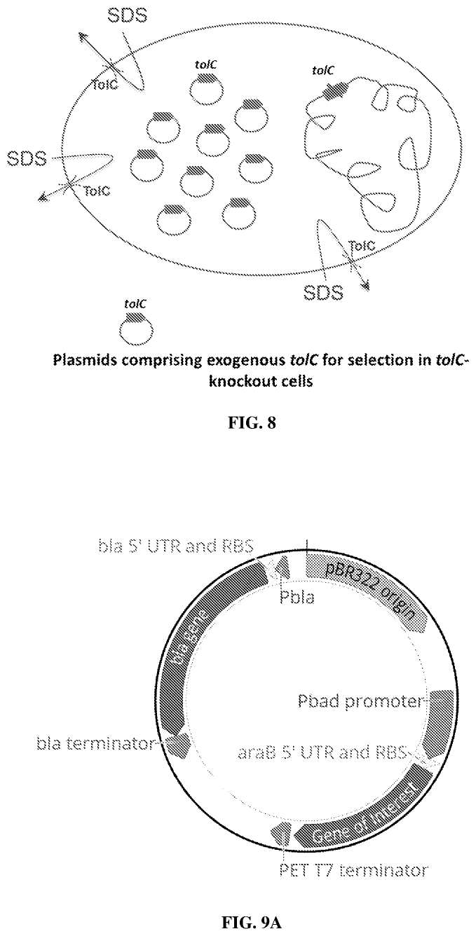

Different promoters were tested in different combinations to assess their ability to drive expression of the gapA marker in the gapA addiction-based plasmids to obtain plasmid yields comparable to or better than a plasmid carrying the bla resistance marker expressed from the constitutive P_{bla} promoter and the bla gene's natural RBS (ARMed control plasmid) (FIG. 7C). GL18-140 (an E. coli strain with the endogenous gapA, endA and recA genes deleted) was used as a background strain for transforming plasmids carrying a gapA marker. GL17-294 (a gapA⁺ strain with the endogenous endA and recA genes deleted) was used as a background strain for transforming plasmid DNA with the bla resistance marker. Each of the plasmids in this Example, in addition to the gapA or bla markers, carried two expression cassettes that would respectively allow expression of the sense and the antisense strands of a dsRNA product.

For each of the strains, plasmid yield (grams of pDNA/gram of biomass) and/or titer (g/L of pDNA) were measured from biomass harvested after growth in either shake flasks (shake flask assay) or in 2 L fed batch fermentations (fed-batch assay). For the shake flask assay, the inoculum for each strain was initially grown in 25 mL of Luria-Broth (LB) medium in a culture flask that had been inoculated with 0.625 mL (2.5% v/v inoculum) of frozen cells (frozen stocks at an optical density (OD₆₀₀) of ˜1) and incubated in a rotary shaker at 30° C. and 300 rpm. After the cells in the first preculture stage reached an OD₆₀₀ of 2, 2.5 mL (2.5% v/v inoculum) of these cells were transferred to the experimental stage flask containing 97.5 mL of defined medium (Korz medium). The flasks in the experimental stage were incubated in a rotary shaker at elevated temperatures (between 37° C. and 45° C.) and 300 rpm for 9 h to enable increased pDNA replication. Carbenicillin (at a final concentration of 50 mg/L in the media) was used to maintain selection pressure for the ARMed strain (GL18-020) while growth in the defined medium served as a natural explicit selection for the unARMed strains. At the end of the assay, cell culture broth from each shake flask was harvested and initially diluted to 2 OD (0.84 g DCW/L). Then, the QIAprep Spin Miniprep Kit high yield protocol was used for pDNA extraction and purification. The pDNA concentration was quantified using a Quant-iT™ PicoGreen™ kit.

For the fed-batch assay, the fermentation process was comprised of an initial batch phase followed by a fed-batch fermentation phase. Both phases were controlled at a pH of 7.05, dissolved oxygen at or above 30%, and a temperature of 30° C. until induction. The pH was controlled with 30% (v/v) ammonium hydroxide and dissolved oxygen was maintained using agitation and oxygen supplementation when needed. At the end of batch phase, glucose feed was initiated on a continuous basis, progressively ramping the feed rate in a linear mode. This fermentation employed a temperature-based induction system to trigger the increase in copy number of the plasmid DNA template once the OD₆₀₀ of the culture reached 60. During this induction period, temperature was elevated (between 37° C. and 45° C.) for copy number amplification. The fermentation was conducted in defined medium (Korz medium) containing salts (such as phosphate and sulfates), trace metals (such as magnesium, iron, calcium, manganese, and zinc) and vitamins (such as thiamine).

For fed-batch fermentations, titers (g/L) or yields (g-pDNA/g-DCW of biomass) of plasmid DNA produced from strains carrying plasmids with different promoter-RBS combinations driving gapA expression, relative to the ARMed GL18-020 strain as a control, are provided in FIGS. 7A-7C and in Table 11.

TABLE 11
Plasmid DNA titers

| | | | | | Shake | |
|---|---|---|---|---|---|---|
| | | | | | Flask | |
| | | | | | Titer | |
| E. coli | | | | Promoter + RBS Sequence | (% of | HCD Ferm. |
| strain | Plasmid | Promoter | RBS | (5′ → 3′) | control) | Titer (g/L) |
| GL18- | pGLA709 | P_{bla} | bla_{rbs} | CCTATTTGTTTATTTTTCTAAATAC | 100% | 1.03 |
| 020 | | | | ATTCAAATATGTATCCGCTCATGA | | |
| | | | | GACAATAACCCTGATAAATGCTTC | | |
| | | | | AATCATGATTGAAAAAGGAAGAGT | | |
| | | | | (SEQ ID NO: 56) | | |
| GL18- | pGLX057 | J23109 | gapA_{rbs} | TTTACAGCTAGCTCAGTCCTAGGG | 74% | 0.70 |
| 188 | | | | ACTGTGCTAGCAACCTTTTATTCAC | | |
| | | | | TAACAAATAGCTGGTGGAATAT | | |
| | | | | (SEQ ID NO: 57) | | |
| GL18- | pGLX059 | J23113 | gapA_{rbs} | CTGATGGCTAGCTCAGTCCTAGGG | 69% | No data |
| 190 | | | | ATTATGCTAGCAACCTTTTATTCAC | | |
| | | | | TAACAAATAGCTGGTGGAATAT | | |
| | | | | (SEQ ID NO: 58) | | |
| GL18- | pGLX060 | J23114 | gapA_{rbs} | TTTATGGCTAGCTCAGTCCTAGGTA | 56% | No data |
| 191 | | | | CAATGCTAGCAACCTTTTATTCACT | | |
| | | | | AACAAATAGCTGGTGGAATAT | | |
| | | | | (SEQ ID NO: 59) | | |
| GL18- | pGLX063 | J23117 | gapA_{rbs} | TTGACAGCTAGCTCAGTCCTAGGG | 50% | 0.94 |
| 194 | | | | ATTGTGCTAGCAACCTTTTATTCAC | | |
| | | | | TAACAAATAGCTGGTGGAATAT | | |
| | | | | (SEQ ID NO: 60) | | |
| GL18- | pGLX015 | P_{bla} | bla_{rbs} | CCTATTTGTTTATTTTTCTAAATAC | 49% | 0.64 |
| 154 | | | | ATTCAAATATGTATCCGCTCATGA | | |
| | | | | GACAATAACCCTGATAAATGCTTC | | |
| | | | | AATCATGATTGAAAAAGGAAGAGT | | |
| | | | | (SEQ ID NO: 56) | | |
| GL18- | pGLX061 | J23115 | gapA_{rbs} | TTTATAGCTAGCTCAGCCCTTGGTA | 45% | No data |
| 192 | | | | CAATGCTAGCAACCTTTTATTCACT | | |
| | | | | AACAAATAGCTGGTGGAATAT | | |
| | | | | (SEQ ID NO: 61) | | |
| GL18- | pGLX058 | J23110 | gapA_{rbs} | TTTACGGCTAGCTCAGTCCTAGGT | 42% | 0.72 |
| 189 | | | | ACAATGCTAGCAACCTTTTATTCAC | | |
| | | | | TAACAAATAGCTGGTGGAATAT | | |
| | | | | (SEQ ID NO: 62) | | |
| GL18- | pGLX056 | J23108 | gapA_{rbs} | CTGACAGCTAGCTCAGTCCTAGGT | 25% | 0.27 |
| 187 | | | | ATAATGCTAGCAACCTTTTATTCAC | | |
| | | | | TAACAAATAGCTGGTGGAATAT | | |
| | | | | (SEQ ID NO: 63) | | |
| GL19- | pGLX098 | J23107 | B0032 | TTTACGGCTAGCTCAGCCCTAGGT | No data | 0.90 |
| 038 | | | | ATTATGCTAGCATGGATCACACAG | | |
| | | | | GAAAGGCCCAT | | |
| | | | | (SEQ ID NO: 64) | | |

### Example 6: Construction of ARMed Plasmids or unARMed Plasmids Employing a tolC-Based Selection

One set of plasmids utilizing tolC-based addiction for plasmid retention in bacterial hosts were constructed to demonstrate that such unARMed plasmids can be used to achieve levels of recombinant protein overexpression that are comparable to plasmids utilizing traditional antibiotic resistance markers (ARMed plasmids) for selection. Additionally, a second set of plasmids utilizing tolC-based addiction for plasmid retention were constructed to demonstrate that such unARMed plasmids can be used to obtain yields of template plasmid DNA comparable to those obtained with ARMed plasmids from E. coli fermentations. Details on the construction of these two sets of plasmids are given below. Additionally, ARMed plasmids were constructed for use as controls.

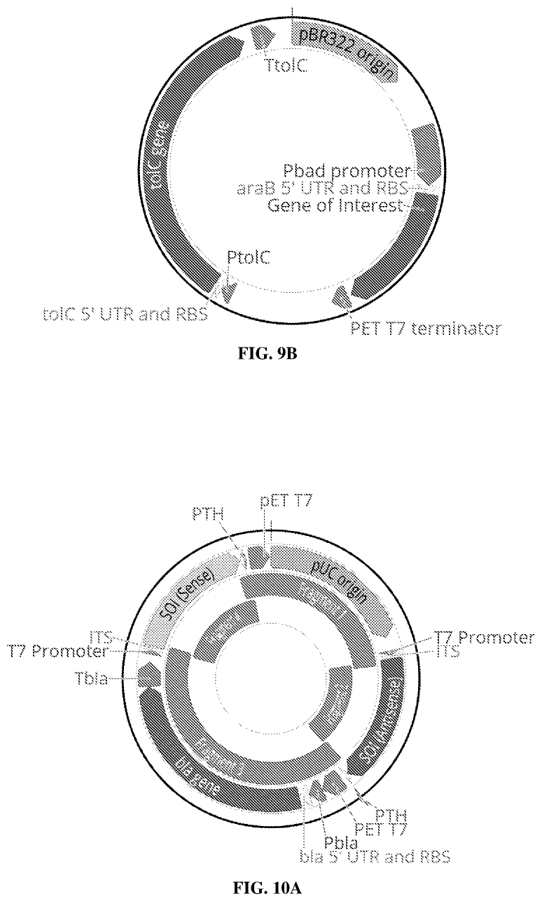

The ARMed expression plasmids described in Table 12 comprise a) an expression cassette for the expression of the bla gene (encoding β-lactamase) as an carbenicillin-resistance marker under the transcriptional and translational control of the Pbla promoter (SEQ ID NO: 20) and the 5′UTR comprising the bla_{rbs} RBS(SEQ ID NO: 33) respectively with the Tbla terminator (Tbla, SEQ ID NO: 49) downstream of the bla gene, b) a pBR322 origin of replication and c) an expression cassette for the expression of recombinant proteins comprising a P_{BAD} promoter (SEQ ID NO: 21) and a 5′UTR comprising the araB_{rbs} RBS (SEQ ID NO: 35) placed upstream of two multi-cloning sites for up to two genes of interest or sequences of interest with a single pET-T7 terminator (SEQ ID NO: 44) downstream of these multi-cloning sites. An example ARMed plasmid template is shown in FIG. 9A.

The unARMed plasmids based on a tolC plasmid addiction system comprised a) an expression cassette for the E. coli tolC gene as an antibiotic-free marker, comprising the native tolC promoter (P_{tolC}, SEQ ID NO: 24) and the 5′UTR comprising the tolC RBS (SEQ ID NO: 34) driving expression of the tolC gene with the TtolC terminator (SEQ ID NO: 48) downstream of the tolC gene, b) a pBR322 origin of replication, c) an expression cassette for the expression of recombinant enzymes comprising a P_{BAD} promoter (SEQ ID NO: 21) and a 5′UTR comprising the araB_{rbs} RBS (SEQ ID NO: 35) placed upstream of two multi-cloning sites for up to two genes of interest or sequences of interest with a single pET-T7 terminator (SEQ ID NO: 44) downstream of these multi-cloning sites. An example unARMed plasmid template is shown in FIG. 9B.

TABLE 12
ARMed and unARMed plasmids expressing recombinant proteins.

| | Selection | Recombinant |
|---|---|---|
| Plasmid | Marker | Protein |
| pGLA590 | bla gene (ARMed) | Enterobacteria phage T7 |
| | | RNA polymerase |
| pGLA678 | bla gene (ARMed) | Aquifex aeolicus |
| | | NDP kinase |
| pGLA680 | bla gene (ARMed) | Thermus thermophilus |
| | | CMP kinase |
| pGLA682 | bla gene (ARMed) | Pyrococcus furiosus |
| | | UMP kinase |
| pGLA684 | bla gene (ARMed) | Thermotoga maritima |
| | | GMP kinase |
| pGLA705 | bla gene (ARMed) | Deinococcus geothermalis |
| | | PP kinase |
| pGLX017 | tolC gene (unARMed) | Thermus thermophilus |
| | | CMP kinase |

Plasmids containing expression cassettes may themselves be the desired products of microbial fermentation and need to have origins of replication that support high-copy-number replication in order to maximize plasmid DNA (pDNA) yield. To demonstrate that such high-copy plasmids can be effectively produced using tolC addiction as the plasmid retention mechanism, several plasmids were constructed that contain the pUC origin of replication and transcription cassettes that enable synthesis of a dsRNA product (GS1).

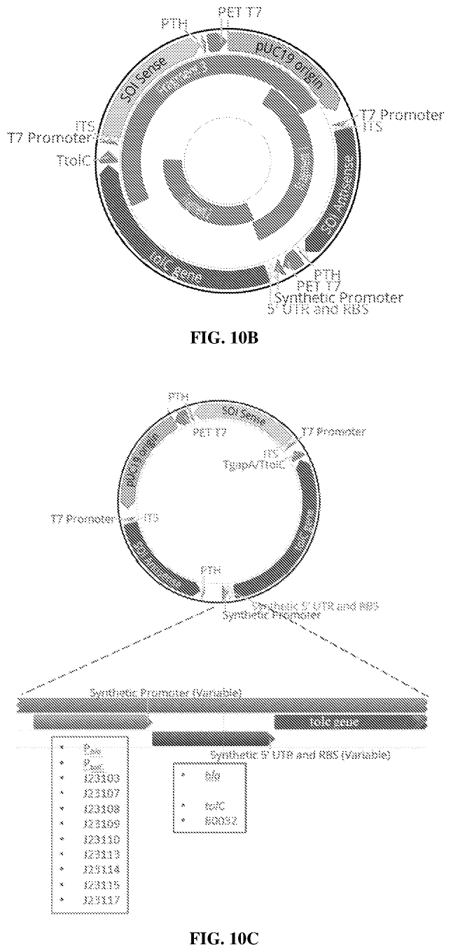

ARMed template plasmids containing the bla gene have a structure, represented by the plasmid map shown in FIG. 10A, consisting of two transcription cassettes, wherein each cassette, a sequence of interest to be transcribed is flanked by the Extended T7 promoter (SEQ ID NO: 23) and ITS (SEQ ID NO: 24) on the 5′ end and the Term 18 dual terminator complex (SEQ ID NO: 37) consisting of the PTH (SEQ ID NO: 47) and pET-T7 (SEQ ID NO: 44) terminators on the 3′ end. The pUC origin of replication is located between the 3′ end of the sense cassette and the 5′ end of the antisense cassette and expression of the bla gene was driven from an expression cassette comprising the Pbla promoter (SEQ ID NO: 20) and the 5′UTR comprising the bla_{rbs} RBS (SEQ ID NO: 33) upstream of the bla gene and the Tbla terminator downstream of it.

UnARMed template plasmids have a structure, as shown in FIG. 10B, that is similar to ARMed template plasmids except for the replacement of the bla gene with the E. coli tolC gene. A variety of promoter and RBSes as described in Table 13 were utilized to vary the level of expression of the tolC gene.

TABLE 13
ARMed and unARMed Template Plasmids for dsRNA Production

| | | Selection | Selection | |
|---|---|---|---|---|
| | Selection | Marker | Marker | dsRNA |
| Plasmid | Marker | Promoter | RBS | Product |
| pGLA709 | bla gene (ARMed) | Pbla | bla | GS1 |
| pGLX144 | tolC gene (unARMed) | J23103 | tolC | GS1 |
| pGLX145 | tolC gene (unARMed) | J23110 | tolC | GS1 |
| pGLX146 | tolC gene (unARMed) | J23113 | tolC | GS1 |
| pGLX147 | tolC gene (unARMed) | J23115 | tolC | GS1 |
| pGLX148 | tolC gene (unARMed) | J23117 | tolC | GS1 |

### Example 7: Development of a tolC-Based Plasmid Addiction System

The endogenous tolC gene was removed from the chromosome of E. coli cells (33B03, a BL21(DE3) derived strain that has the endogenous tolC gene deleted) using lambda red recombination. The endogenous tolC gene was replaced with a tolC gene marker that can be selected for by growth in the presence of sodium dodecyl sulfate (SDS) to prepare strain GL18-172. Cells that acquired the desired ΔtolC::tolC replacement were confirmed by PCR amplification and sequencing of the corresponding locus of the genome.

An isolated single colony of E. coli that was confirmed to contain the ΔtolC::tolC chromosomal modification was saved as strain GL18-172 (Table 14) and characterized for its ability to grow in Luria Broth (LB) medium comprising 50 mg/mL SDS. For comparison, the strains described in Table 14 were also grown in LB medium comprising either 0 or 50 mg/mL SDS (FIG. 11).

TABLE 14
Strains described in Example 7

| | Baseline | | | Relevant |
|---|---|---|---|---|
| | Chromosome | | Plasmid | Plasmid |
| Strain | Genotype | Plasmid | Selection | Genotype |
| 33B03 | ΔtolC | None | N/A | N/A |
| GL17-086 | BL21(DE3) | None | N/A | N/A |
| GL17-277 | BL21(DE3) | pGLA680 | bla (ARMed) | pMB1-ori P_{BAD}-His- |
| | | | | Tthcmk-T_{T7} P_{bla}-bla-T_{bla} |
| GL18-172 | 33B03 (ΔtolC ) | pGLX017 | tolC (unARMed) | pMB1-ori P_{BAD}-His- |
| | | | | Tthcmk-T_{T7} P_{tolC}-tolC-T_{tolC} |

33B03 (ΔtolC, i.e., tolC-deficient), GL17-086 (endogenous tolC⁺), GL17-277 (endogenous tolC⁺ complemented with bla⁺ plasmid, ARMed, i.e., resistant to carbenicillin), and GL18-172 (ΔtolC complemented with tolC⁺ plasmid, unARMed) strains were individually grown overnight in LB media at 37° C. with 250 rpm shaking.

On the next day, six shake flasks containing 10 mL of LB media were inoculated as follows: (1) 100 μL of overnight culture of GL17-086 (no SDS); (2) 100 μL of overnight culture of GL17-086 in presence of 50 mg/L SDS; (3) 100 μL of overnight culture of GL17-277 in presence of carbenicillin (no SDS); (4) 100 μL of overnight culture of 33B03 (no SDS); (5) 100 μL of overnight culture of 33B03 in presence of 50 mg/L SDS; and (6) 100 μL of overnight culture of GL18-172 in presence of 50 mg/L SDS. Shake flasks were then incubated at 37° C. with 250 rpm shaking. Samples were taken periodically for OD₆₀₀ measurement.

FIG. 11 show the growth of each of the six shake flask cultures over a period of 6.5 hours. GL17-086 (endogenous tolC⁺) grew in both of (1), i.e., absence of SDS; and (2), i.e., presence of 50 mg/L SDS. GL17-277 grew robustly in (3), i.e., presence of carbenicillin without SDS. Strain 33B03 (ΔtolC) was grew robustly in (4), i.e., absence of SDS. Strain 33B03 (ΔtolC) was unable to grow in (5), i.e., presence of 50 mg/L SDS, demonstrating that removal of endogenous tolC significantly reduces the ability of E. coli to grow in the presence of 50 mg/L SDS. However, the introduction of a plasmid comprising exogenous tolC into a ΔtolC strain (UnARMed GL18-172) was able to rescue the ability of E. coli to grow in (6), i.e., the presence of 50 mg/L SDS.

These data demonstrate that that a plasmid carrying tolC can successfully rescue growth of a tolC deficient bacterial cell in media containing a surfactant such as SDS.

### Example 8: Production of Plasmid DNA Using a tolC-Based Plasmid Addiction System

Production of plasmid DNA as a desired product via fermentation at a high yield and titer requires the stable maintenance and propagation of high copy plasmids in microbial hosts. The use of the tolC plasmid addiction system to maintain a high-copy plasmid with a pUC origin of replication in E. coli provided good yields of a similar high-copy pUC plasmid employing a bla antibiotic resistance marker for plasmid maintenance. The pUC origin of replication allows temperature-inducible amplification of plasmid copy number.

Different promoters were tested in different combinations to assess their ability to drive expression of the tolC gene in tolC addiction-based plasmids to obtain plasmid yields comparable to a plasmid carrying the bla resistance marker expressed from the constitutive P_{b}/a promoter and the bla gene's natural RBS (ARMed control plasmid). An E. coli strain with the endogenous tolC, endA and recA genes deleted was used as a background strain for transforming plasmids carrying a tolC marker. Each of the plasmids in this Example, in addition to the tolC or bla markers, carried two expression cassettes that would respectively allow expression of the sense and the antisense strands of a dsRNA product (GS1).

For each of the strains, plasmid yield (grams of pDNA per gram of biomass) and/or titer (g/L of pDNA) were measured from biomass harvested after growth in either shake flasks (shake flask assay) or in 2 L fed batch fermentations (fed-batch assay). For the shake flask assay, the inoculum for each strain was initially grown in 25 mL of Luria-Broth (LB) medium in a culture flask that had been inoculated with 0.625 mL (2.5% v/v inoculum) of frozen cells (frozen stocks at an optical density (OD₆₀₀) of ˜1) and incubated in a rotary shaker at 30° C. and 300 rpm. After the cells in the first preculture stage reached an OD₆₀₀ of 2, 2.5 mL (2.5% v/v inoculum) of these cells were transferred to the experimental stage flask containing 97.5 mL of defined medium, containing 25 g/L of glycerol as a carbon source. The flasks in the experimental stage were incubated in a rotary shaker at elevated temperatures (between 37° C. and 45° C.) and 300 rpm for 9 h to enable increased pDNA replication. Carbenicillin (at a final concentration of 50 mg/L in the media) was used to maintain selection pressure for the ARMed strain (GL18-020) while addition of 50 mg/L of sodium dodedcyl sulfate (SDS) in the defined medium served as the selection for the unARMed strains. At the end of the assay, cell culture broth from each shake flask was harvested and initially diluted to 2 OD (0.84 g DCW/L). Then, the QIAprep Spin Miniprep Kit high yield protocol was used for pDNA extraction and purification. The pDNA concentration was quantified using a Quant-iT™ PicoGreen™ kit.

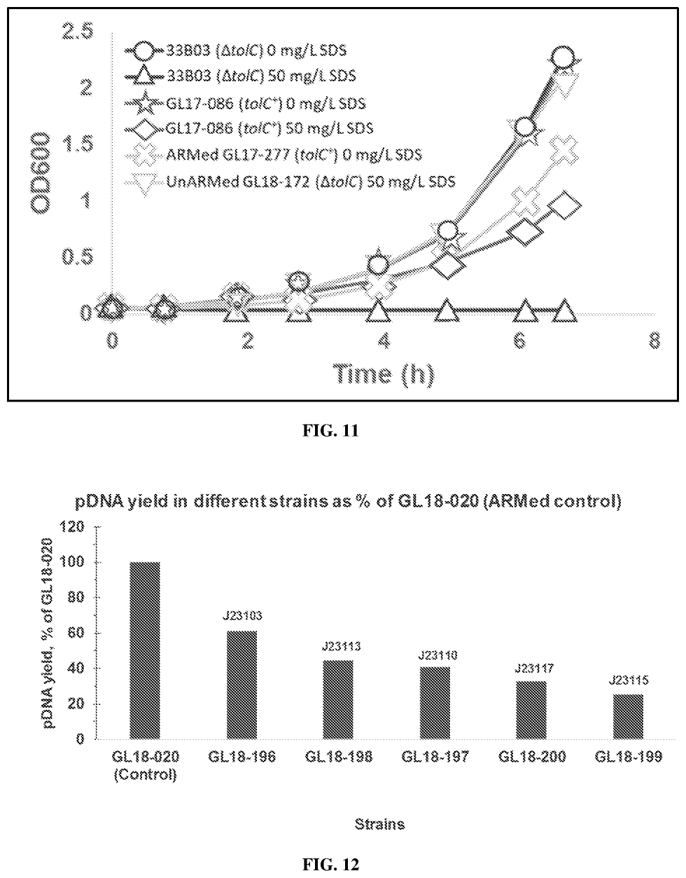

Yields of plasmid DNA produced by the tolC plasmid-addicted UnARMed strains are provided in FIG. 12 and in Table 15.

TABLE 15
Plasmid DNA titers (grams/liter).

| E. coli | | | | Shake Flask Titer |
|---|---|---|---|---|
| strain | Plasmid | Promoter | RBS | (% of GL18-020 control) |
| GL18-020 | pGLA709 | P_{bla} | bla | 100% |
| GL18-196 | pGLX144 | J23103 | tolC | ~60% |
| GL18-198 | pGLX146 | J23113 | tolC | ~50% |
| GL18-197 | pGLX145 | J23110 | tolC | ~40% |
| GL18-200 | pGLX148 | J23117 | tolC | ~35% |
| GL18-199 | pGLX147 | J23115 | tolC | ~30% |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A microbial cell lacking or having decreased expression of an endogenous glycolytic gene that encodes a glycolytic enzyme, wherein the microbial cell comprises a nucleic acid construct comprising an expression cassette that encodes a recombinant glycolytic enzyme, and wherein the microbial cell can grow in a defined medium and/or a complex medium.

2. The microbial cell of claim 1, wherein the microbial cell cannot grow in the defined medium and/or the complex medium without the nucleic acid construct.

3. The microbial cell of claim 1 or 2, wherein the recombinant glycolytic enzyme has the same enzymatic activity as the endogenous glycolytic gene.

4. The microbial cell of any one of claims 1-3, wherein the chromosomal DNA of the microbial cell comprises a genetic modification of the endogenous gene or an element controlling the expression of the endogenous gene that decreases the expression of the glycolytic enzyme, optionally wherein the genetic modification is a mutation, insertion or deletion.

5. The microbial cell of any one of claims 1-4, wherein the nucleic acid construct is a plasmid, a vector, a cosmid, a bacterial artificial chromosome, a yeast artificial chromosome, a bacteriophage, a viral vector or any other.

6. The microbial cell of any one of claims 1-5, wherein the endogenous glycolytic gene encodes a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase.

7. The microbial cell of any one of claims 1-6, wherein the recombinant glycolytic enzyme is a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase.

8. The microbial cell of any one of claims 1-7, wherein the endogenous glycolytic gene encodes a glycolytic enzyme having glyceraldehyde 3-phosphate dehydrogenase (GAPDH) activity, and wherein the recombinant glycolytic enzyme has GAPDH activity.

9. The microbial cell of any one of claims 1-8, wherein the endogenous glycolytic gene encodes a glyceraldehyde 3-phosphate dehydrogenase, and wherein the recombinant glycolytic enzyme is a glyceraldehyde 3-phosphate dehydrogenase.

10. The microbial cell of claim 9, wherein the glyceraldehyde 3-phosphate dehydrogenase comprises an amino acid sequence of SEQ ID NO: 50.

11. The microbial cell of any one of claims 1-10, wherein the microbial cell is a prokaryotic or eukaryotic cell, optionally wherein the microbial cell is a bacterial cell or a yeast cell.

12. The microbial cell of any one of claims 1-11, wherein the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell.

13. The microbial cell of any one of claims 1-12, wherein the microbial cell is an Escherichia coli (E. coli) cell, the endogenous glycolytic gene is gapA, and the recombinant glycolytic enzyme is a glyceraldehyde 3-phosphate dehydrogenase.

14. The microbial cell of any one of claims 1-13, wherein the complex media is Luria Broth (LB), Terrific Broth, Super Optimal broth with Catabolite repression (SOC media), or any derivative thereof.

15. The microbial cell of any one of claims 1-14, wherein the defined medium is Korz broth, M9 minimal media, or any derivative thereof.

16. The microbial cell of any one of claims 1-15, wherein the nucleic acid construct further comprises a replicon comprising an origin of replication and its control elements.

17. The microbial cell of claim 16, wherein the replicon is of bacterial origin.

18. The microbial cell of claim 16, wherein the replicon is the ColE1 replicon, the pUC replicon or is derived from the ColE1, pBR322, pUC, R6K, p15a or pSC101 replicon.

19. The microbial cell of any one of claims 1-18, wherein the expression cassette that encodes a recombinant glycolytic enzyme comprises a promoter operably linked to the coding sequence for the recombinant glycolytic enzyme.

20. The microbial cell of claim 19, wherein the promoter comprises a nucleic acid sequence set forth in any one of SEQ ID NO: 1-23.

21. The microbial cell of claim 19, wherein the promoter consists of a nucleic acid sequence set forth in any one of SEQ ID NO: 1-23.

22. The microbial cell of claim 19, wherein the expression cassette that encodes a recombinant glycolytic enzyme further comprises an initial transcription sequence (ITS) upstream of the coding sequence for the recombinant glycolytic enzyme.

23. The microbial cell of claim 22, wherein the ITS comprises a nucleic acid sequence set forth in SEQ ID NO: 24.

24. The microbial cell of claim 22, wherein the ITS consists of a nucleic acid sequence set forth in SEQ ID NO: 24.

25. The microbial cell of any one of claims 19-24, wherein the expression cassette that encodes a recombinant glycolytic enzyme further comprises a 5′UTR comprising a ribosome binding site (RBS) placed upstream of the coding sequence for the recombinant glycolytic enzyme and one or more terminators downstream of the coding sequence for the recombinant glycolytic enzyme.

26. The microbial cell of claim 25, wherein the RBS comprises a nucleic acid sequence set forth in any one of SEQ ID NO: 25-35.

27. The microbial cell of claim 25, wherein the RBS consists of a nucleic acid sequence set forth in any one of SEQ ID NO: 25-35.

28. The microbial cell of any one of claims 25-27, wherein the one or more terminators comprises a nucleic acid sequence set forth in any one of SEQ ID NO: 36-49.

29. The microbial cell of any one of claims 25-27, wherein the one or more terminators consists of a nucleic acid sequence set forth in any one of SEQ ID NO: 36-49.

30. The microbial cell of any one of claims 1-29, wherein the nucleic acid construct further comprises an expression cassette comprising a sequence of interest, wherein the sequence of interest encodes a RNA product, peptide product or protein product.

31. The microbial cell of claim 30, wherein the RNA product is a messenger RNA, siRNA, microRNA, guide RNA, a sense strand of a double-stranded RNA, or an antisense strand of a double-stranded RNA.

32. The microbial cell of claim 30 or 31, wherein the nucleic acid construct comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

33. The microbial cell of any one of claims 30-32, wherein the expression cassette comprising a sequence of interest further comprises a promoter operably linked to the sequence of interest.

34. The microbial cell of claim 33, wherein the promoter comprises a nucleic acid sequence set forth in any one of SEQ ID NO: 1-23.

35. The microbial cell of claim 33, wherein the promoter consists of a nucleic acid sequence set forth in any one of SEQ ID NO: 1-23.

36. The microbial cell of any one of claims 30-35, wherein the expression cassette comprising a sequence of interest further comprises one or more of the sequence elements selected from the group consisting of: a promoter, an initial transcription sequence, a ribosome binding site, a restriction endonuclease site, and a terminator.

37. The microbial cell of any one of claims 1-36, wherein the microbial cell does not comprise an antibiotic resistance gene.

38. A plasmid addiction system comprising:
(i) a microbial cell comprising a genetic modification of a glycolytic gene that encodes an endogenous glycolytic enzyme, wherein the genetic modification reduces or abolishes the expression of the endogenous glycolytic enzyme; and
(ii) a plasmid comprising an expression cassette that encodes a recombinant glycolytic enzyme;
wherein the microbial cell cannot grow or propagate without incorporation of the plasmid.

39. The plasmid addiction system of claim 38, wherein the genetic modification comprises a mutation, insertion or deletion within the glycolytic gene or a control element of the glycolytic gene, optionally wherein the control element is a promoter or a ribosome binding site.

40. The plasmid addiction system of claim 38 or 39, wherein the recombinant glycolytic enzyme has the same enzymatic activity as the endogenous glycolytic enzyme.

41. The plasmid addiction system of any one of claims 38-40, wherein the microbial cell can grow in a defined medium and/or a complex medium if the plasmid is incorporated into the cell.

42. The plasmid addiction system of any one of claims 38-41, wherein the modified glycolytic gene encodes a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase.

43. The plasmid addiction system of any one of claims 38-42, wherein the recombinant glycolytic enzyme is a hexokinase, a glucose phosphate isomerase, a phosphofructokinase, an aldolase, a triosephosphate isomerase, a phosphoglycerate kinase, an enolase, a pyruvate kinase, a phosphoenolpyruvate carboxylase, a pyruvate carboxylase or a glyceraldehyde 3-phosphate dehydrogenase.

44. The plasmid addiction system of any one of claims 38-43, wherein the modified glycolytic gene encodes a glycolytic enzyme having glyceraldehyde 3-phosphate dehydrogenase (GAPDH) activity, and wherein the recombinant glycolytic enzyme has GAPDH activity.

45. The plasmid addiction system of any one of claims 38-44, wherein the modified glycolytic gene encodes a glyceraldehyde 3-phosphate dehydrogenase, and wherein the recombinant glycolytic enzyme is a glyceraldehyde 3-phosphate dehydrogenase.

46. The plasmid addiction system of 45, wherein the glyceraldehyde 3-phosphate dehydrogenase comprises an amino acid sequence of SEQ ID NO: 50.

47. The plasmid addiction system of any one of claims 38-46, wherein the microbial cell is a prokaryotic or eukaryotic cell, optionally wherein the microbial cell is a bacterial cell or a yeast cell.

48. The plasmid addiction system of any one of claims 38-46, wherein the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell.

49. The plasmid addiction system of any one of claims 38-48, wherein the microbial cell is an Escherichia coli (E. coli) cell, the inactivated glycolytic gene is gapA, and the recombinant glycolytic enzyme is a glyceraldehyde 3-phosphate dehydrogenase.

50. The plasmid addiction system of any one of claims 41-49, wherein the complex medium is Luria Broth (LB), Terrific Broth, Super Optimal broth with Catabolite repression (SOC media), or any derivative thereof.

51. The plasmid addiction system of any one of claims 41-50, wherein the defined medium is Korz broth, M9 minimal media, or any derivative thereof.

52. The plasmid addiction system of any one of claims 41-51, wherein the plasmid comprises a replicon comprising an origin of replication and its control elements that allows replication of the plasmid in the microbial cell, optionally wherein the replicon is the ColE1 replicon, the pUC replicon or is derived from the ColE1, pUC, pBR322, R6K, p15a or pSC101 replicon.

53. The plasmid addiction system of any one of claims 41-52, wherein the expression cassette that encodes a recombinant glycolytic enzyme comprises a promoter operably linked to the coding sequence for the recombinant glycolytic enzyme.

54. The plasmid addiction system of claim 53, wherein the promoter comprises a nucleic acid sequence set forth in any one of SEQ ID NO: 1-23.

55. The plasmid addiction system of claim 53, wherein the promoter consists of a nucleic acid sequence set forth in any one of SEQ ID NO: 1-23.

56. The plasmid addiction system of claims 53-55, wherein the expression cassette that encodes a recombinant glycolytic enzyme further comprises an initial transcription sequence (ITS) upstream of the coding sequence for the recombinant glycolytic enzyme.

57. The plasmid addiction system of claim 56, wherein the ITS comprises a nucleic acid sequence set forth in SEQ ID NO: 24.

58. The plasmid addiction system of claim 56, wherein the ITS consists of a nucleic acid sequence set forth in SEQ ID NO: 24.

59. The plasmid addiction system of any one of claims 53-58, wherein the expression cassette that encodes a recombinant glycolytic enzyme further comprises a 5′UTR comprising a ribosome binding site (RBS) placed upstream of the coding sequence for the recombinant glycolytic enzyme and one or more terminators downstream of the coding sequence for the recombinant glycolytic enzyme.

60. The plasmid addiction system of claim 59, wherein the RBS comprises a nucleic acid sequence set forth in any one of SEQ ID NO: 25-35.

61. The plasmid addiction system of claim 59, wherein the RBS consists of a nucleic acid sequence set forth in any one of SEQ ID NO: 25-35.

62. The plasmid addiction system of any one of claims 59-61, wherein the one or more terminators comprises a nucleic acid sequence set forth in any one of SEQ ID NO: 36-49.

63. The plasmid addiction system of any one of claims 59-61, wherein the one or more terminators consists of a nucleic acid sequence set forth in any one of SEQ ID NO: 36-49.

64. The plasmid addiction system of any one of claims 59-63, wherein the plasmid further comprises an expression cassette comprising a sequence of interest, wherein the sequence of interest encodes a RNA product, peptide product or protein product.

65. The plasmid addiction system of claim 64, wherein the RNA product is a messenger RNA, siRNA, microRNA, guide RNA, a sense strand of a double-stranded RNA, or an antisense strand of a double-stranded RNA.

66. The plasmid addiction system of claim 64 or 65, wherein the plasmid comprises two expression cassettes comprising a sequence of interest, wherein the first expression cassette comprises a first sequence of interest that encodes a sense strand of a double-stranded RNA, and wherein the second expression cassette comprises a second sequence of interest that encodes an antisense strand of the double-stranded RNA.

67. The plasmid addiction system of any one of claims 64-66, wherein the expression cassette(s) comprising a sequence of interest further comprise a promoter operably linked to the sequence of interest.

68. The plasmid addiction system of claim 67, wherein the promoter comprises a nucleic acid sequence set forth in any one of SEQ ID NO: 1-23.

69. The plasmid addiction system of claim 67, wherein the promoter consists of a nucleic acid sequence set forth in any one of SEQ ID NO: 1-23.

70. The plasmid addiction system of any one of claims 64-69, wherein the expression cassette comprising a sequence of interest further comprises one or more of the sequence elements selected from the group consisting of: a promoter, an initial transcription sequence, a ribosome binding site, a restriction endonuclease site, and a terminator.

71. The plasmid addiction system of any one of claims 38-63, wherein the plasmid further comprises one or more multicloning sites (MCSs) or unique restriction endonuclease digestion sites.

72. The plasmid addiction system of any one of claims 38-71, wherein the plasmid does not comprise an antibiotic resistance gene.

73. A nucleic acid construct comprising an expression cassette comprising a gene encoding an enzyme having glyceraldehyde 3-phosphate dehydrogenase (GAPDH) activity and
(i) one or more multiple cloning sites, and/or
(ii) an expression cassette comprising a sequence of interest encoding an RNA product, peptide product or protein product.

74. The nucleic acid construct of claim 73, wherein the nucleic acid construct is a plasmid, a vector, a cosmid, a bacterial artificial chromosome, a yeast artificial chromosome, a bacteriophage, a viral vector or any other.

75. The nucleic acid construct of claim 73 or 74, wherein the gene encoding an enzyme having GAPDH activity is a microbial gapA gene.

76. The nucleic acid construct of any one of claims 73-75, wherein the enzyme having GAPDH activity comprises an amino acid sequence of SEQ ID NO: 50.

77. The nucleic acid construct of any one of claims 73-76, wherein the nucleic acid construct comprises a first sequence of interest and a second sequence of interest, optionally wherein a first expression cassette comprises the first sequence of interest and a second expression cassette comprises the second sequence of interest.

78. The nucleic acid construct of claim 77, wherein the first sequence of interest encodes a sense strand of a double-stranded RNA product, and the second sequence of interest encodes an antisense strand of a double-stranded RNA product.

79. The nucleic acid construct of any one of claims 73-78, wherein any one of the expression cassettes further comprises a promoter and/or terminator.

80. The nucleic acid construct of claim 79, wherein the promoter comprises a nucleic acid sequence set forth in any one of SEQ ID NO: 1-23.

81. The nucleic acid construct of claim 79, wherein the promoter consists of a nucleic acid sequence set forth in any one of SEQ ID NO: 1-23.

82. The nucleic acid construct of any one of claims 79-81, wherein the promoter is operably linked to an initial transcription sequence (ITS).

83. The nucleic acid construct of claim 82, wherein the ITS comprises a nucleic acid sequence set forth in SEQ ID NO: 24.

84. The nucleic acid construct of claim 82, wherein the ITS consists of a nucleic acid sequence set forth in SEQ ID NO: 24.

85. The nucleic acid construct of any one of claims 79-81, wherein the promoter is operably linked to a 5′UTR comprising a ribosome binding site (RBS).

86. The nucleic acid construct of claim 85, wherein the RBS comprises a nucleic acid sequence set forth in SEQ ID NO: 25-35.

87. The nucleic acid construct of claim 85, wherein the RBS consists of a nucleic acid sequence set forth in SEQ ID NO: 25-35.

88. A method comprising culturing the microbial cell of any one of claims 1-37 in the absence of an antibiotic under condition sufficient to produce the nucleic acid construct.

89. The method of claim 88, wherein the method produces at least 50% of the total amount of the nucleic acid construct as produced by a control microbial cell comprising an antibiotic resistance marker gene.

90. The method of claim 88, wherein the method produces at least 90% of the total amount of the nucleic acid construct as produced by a control microbial cell comprising an antibiotic resistance marker gene.

91. A method comprising culturing the microbial cell of any one of claims 16-37 in the absence of an antibiotic under condition sufficient to produce the RNA product, peptide product or protein product.

92. The method of claim 91, wherein the method produces at least 50% of the total amount of the RNA product, peptide product or protein product as produced by a control microbial cell comprising an antibiotic resistance marker gene.

93. The method of claim 91 or 92, wherein the method produces at least 90% of the total amount of the RNA product, peptide product or protein product as produced by a control microbial cell comprising an antibiotic resistance marker gene.

94. A method comprising:
delivering to a microbial cell a vector comprising a gene encoding glyceraldehyde 3-phosphate dehydrogenase,
wherein the microbial cell comprises a genetically modified gene that encodes glyceraldehyde 3-phosphate dehydrogenase, optionally wherein the genetic modification comprises a mutation, insertion or deletion within the gene that encodes glyceraldehyde 3-phosphate dehydrogenase or a control element of the gene, optionally wherein the control element is a promoter or a ribosome binding site.

95. The method of claim 94, further comprising culturing the microbial cell in defined medium or in complex medium.

96. The method of claim 95, wherein the complex medium is Luria Broth (LB), Terrific Broth, Super Optimal broth with Catabolite repression (SOC media), or any derivative thereof.

97. The method of claim 95, wherein the defined medium is Korz broth, M9 minimal media, or any derivative thereof.

98. A kit comprising:
(i) the nucleic acid construct of any one of claims 73-87; and
(ii) a plurality of microbial cells comprising a genetically modified gene that encodes glyceraldehyde 3-phosphate dehydrogenase, optionally wherein the genetic modification comprises a mutation, insertion or deletion.

99. A kit comprising:
(i) a plasmid comprising an expression cassette that encode a recombinant glycolytic enzyme; and
(ii) a plurality of microbial cells comprising a genetic modification of a gene that encodes a glycolytic enzyme, optionally wherein the genetic modification comprises a mutation, insertion or deletion within the glycolytic gene or a control element of the glycolytic gene, further optionally wherein the control element is a promoter or a ribosome binding site.

100. A kit comprising a plurality of microbial cells of any one of claims 1-37.

101. The kit of any one of claims 98-100, wherein the plurality of microbial cells are lyophilized or frozen in a cryoprotectant.

102. A microbial cell lacking or having decreased expression of an endogenous gene that encodes an outer membrane efflux protein, wherein the microbial cell comprises a nucleic acid construct comprising an expression cassette that encodes a recombinant outer membrane efflux protein and an expression cassette that encodes a sequence of interest, and wherein the sequence of interest is expressed when the microbial cell is grown in the presence of a threshold level of a surfactant.

103. The microbial cell of claim 102, wherein the recombinant outer membrane efflux protein has the same enzymatic activity as the endogenous gene that encodes an outer membrane efflux protein.

104. The microbial cell of claim 102 or 103, wherein the chromosomal DNA of the microbial cell comprises a genetic modification of the endogenous gene or an element controlling the expression of the endogenous gene that decreases the expression of the outer membrane efflux protein.

105. The microbial cell of any one of claims 102-104, wherein the endogenous gene encodes a tolC, FusA, mexA, mexB, oprM, PpF1, SepA, SepB, SepC, SmeC, OpmE, OpmD, OpmB, or bepC protein.

106. The microbial cell of any one of claims 102-105, wherein the outer membrane efflux protein is a tolC, FusA, mexA, mexB, oprM, PpF1, SepA, SepB, SepC, SmeC, OpmE, OpmD, OpmB, or bepC protein.

107. The microbial cell of any one of claims 102-106, wherein the endogenous gene encodes a tolC protein, and wherein the recombinant outer membrane efflux protein is a recombinant tolC protein.

108. The microbial cell of claim 107, wherein the recombinant tolC protein comprises an amino acid sequence of SEQ ID NO: 50.

109. The microbial cell of any one of claims 102-108, wherein the microbial cell is a bacterial cell or a yeast cell, optionally wherein the microbial cell is an Escherichia coli (E. coli), Bacillus subtilis (B. subtilis), Pseudomonas aeruginosa (P. aeruginosa), Staphylococcus aureus (S. aureus), Streptococcus pneumoniae (S. pneumoniae), Mycobacterium tuberculosis (M. tuberculosis), Mycobacterium leprae (M. leprae), Mycobacterium smegmatis (M. smegmatis), Saccharomyces cerevisiae (S. cerevisiae), Yarrowia lipolytica (Y. lipolytica), Pichia pastoris (P. pastoris), or Trichoderma reesie (T. reesie) cell.

110. The microbial cell of any one of claims 102-109, wherein the threshold level of the surfactant is a concentration of surfactant that halts cell growth and/or promotes cell death in a control microbial cell, optionally wherein the control microbial cell lacks or has decreased expression of an endogenous gene that encodes an outer membrane efflux protein and does not comprise a nucleic acid construct comprising an expression cassette that encodes a recombinant outer membrane efflux protein.

111. The microbial cell of any one of claims 102-110, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

112. A plasmid addiction system comprising:
(i) a microbial cell comprising a genetic modification of a gene that encodes an outer membrane efflux protein, wherein the genetic modification reduces or abolishes the expression of the endogenous outer membrane efflux protein; and
(ii) a plasmid comprising an expression cassette that encodes a recombinant outer membrane efflux protein;
wherein the microbial cell cannot grow or propagate in a medium containing a threshold level of surfactant without incorporation of the plasmid.

113. The plasmid addiction system of claim 112, wherein the microbial cell can grow and propagate in a medium containing a surfactant if the plasmid is incorporated into the cell.

114. The plasmid addiction system of claim 112 or 113, wherein the modified gene encodes a tolC, FusA, mexA, mexB, oprM, PpF1, SepA, SepB, SepC, SmeC, OpmE, OpmD, OpmB, or bepC protein.

115. The plasmid addiction system of any one of claims 112-114, wherein the recombinant outer membrane efflux protein is a tolC, FusA, mexA, mexB, oprM, PpF1, SepA, SepB, SepC, SmeC, OpmE, OpmD, OpmB, or bepC protein.

116. The plasmid addiction system of any one of claims 112-115, wherein the threshold level of the surfactant is a concentration of surfactant that halts cell growth and/or promotes cell death in a control microbial cell, optionally wherein the control microbial cell lacks or has decreased expression of an endogenous gene that encodes an outer membrane efflux protein and does not comprise a nucleic acid construct comprising an expression cassette that encodes a recombinant outer membrane efflux protein.

117. The plasmid addiction system of any one of claims 112-116, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.

118. A method comprising culturing the microbial cell of any one of claims 102-111 in the presence of a threshold level of a surfactant and the absence of an antibiotic under conditions sufficient to produce the nucleic acid construct.

119. A method comprising:
delivering to a microbial cell a vector comprising a gene encoding tolC and a gene expressing a sequence of interest,
wherein the microbial cell comprises a genetically modified tolC gene, optionally wherein the genetic modification comprises a mutation, insertion or deletion within the tolC gene or a control element of the tolC gene, further optionally wherein the control element is a promoter or a ribosome binding site.

120. The method of claim 119, wherein the threshold level of the surfactant is a concentration of surfactant that halts cell growth and/or promotes cell death in a control microbial cell, optionally wherein the control microbial cell lacks or has decreased expression of an endogenous gene that encodes an outer membrane efflux protein and does not comprise a nucleic acid construct comprising an expression cassette that encodes a recombinant outer membrane efflux protein.

121. The method of claim 119 or 120, wherein the surfactant is sodium dodecyl sulfate (SDS), cetyl trimethylammonium bromide, Triton X-100, 3 [(3 cholamidopropyl)dimethylammonio]-1-propanesulfonate (CHAPS), nonyl phenoxypolyethoxylethanol (NP-40), octyl thioglucoside, octyl glucoside or dodecyl maltoside.
